SEQUENCE LISTING (16 sequences)

SEQ ID NO: 1 - DNA, 19 bp, synthetic construct
  source 1..19: mol_type = other DNA
  misc_feature 1..19: note = BCLAF1 (exon 10-11) forward primer
caggagttagccgaccacg

SEQ ID NO: 2 - DNA, 23 bp, synthetic construct
  source 1..23: mol_type = other DNA
  misc_feature 1..23: note = BCLAF1 (exon 10-11) reverse primer
gtttggaccagtatttgtcccag

SEQ ID NO: 3 - DNA, 23 bp, synthetic construct
  source 1..23: mol_type = other DNA
  misc_feature 1..23: note = BCLAF1 (exon 10-11) probe
aaccttttttcgaattagaggca

SEQ ID NO: 4 - DNA, 20 bp, synthetic construct
  source 1..20: mol_type = other DNA
  misc_feature 1..20: note = BCLAF1 (exon 10-12) forward primer
tgcaggagttagccgaccac

SEQ ID NO: 5 - DNA, 20 bp, synthetic construct
  source 1..20: mol_type = other DNA
  misc_feature 1..20: note = BCLAF1 (exon 10-12) reverse primer
ttggcccaataatccacacc

SEQ ID NO: 6 - DNA, 20 bp, synthetic construct
  source 1..20: mol_type = other DNA
  misc_feature 1..20: note = BCLAF1 (exon 10-12) probe
aacctttcatgacgacagag

SEQ ID NO: 7 - DNA, 29 bp, synthetic construct
  source 1..29: mol_type = other DNA
  misc_feature 1..29: note = S6K (exon 6-7) forward primer
ttatgcagttagaaagagagggaatattt

SEQ ID NO: 8 - DNA, 20 bp, synthetic construct
  source 1..20: mol_type = other DNA
  misc_feature 1..20: note = S6K (exon 6-7) reverse primer
ccccaaagccatggagattt

SEQ ID NO: 9 - DNA, 27 bp, synthetic construct
  source 1..27: mol_type = other DNA
  misc_feature 1..27: note = S6K (exon 6-7) probe
tggaagacactgcctgcttttacttgg

SEQ ID NO: 10 - DNA, 29 bp, synthetic construct
  source 1..29: mol_type = other DNA
  misc_feature 1..29: note = S6K (exon 6-8) forward primer
ttatgcagttagaaagagagggaatattt

SEQ ID NO: 11 - DNA, 26 bp, synthetic construct
  source 1..26: mol_type = other DNA
  misc_feature 1..26: note = S6K (exon 6-8) reverse primer
gattctttgcatagtccaaagtctgt

SEQ ID NO: 12 - DNA, 27 bp, synthetic construct
  source 1..27: mol_type = other DNA
  misc_feature 1..27: note = S6K (exon 6-8) probe
tggaagacactgcctggtcatgtgaaa

SEQ ID NO: 13 - DNA, 25 bp, synthetic construct
  source 1..25: mol_type = other DNA
  misc_feature 1..25: note = BCLxS forward primer
gagctttgaacaggatacttttgtg

SEQ ID NO: 14 - DNA, 20 bp, synthetic construct
  source 1..20: mol_type = other DNA
  misc_feature 1..20: note = BCLxS reverse primer
gaagagtgagcccagcagaa

SEQ ID NO: 15 - DNA, 21 bp, synthetic construct
  source 1..21: mol_type = other DNA
  misc_feature 1..21: note = BCLxL forward primer
gatggccacttacctgaatga

SEQ ID NO: 16 - DNA, 20 bp, synthetic construct
  source 1..20: mol_type = other DNA
  misc_feature 1..20: note = BCLxL reverse primer
tgctgcattgttcccataga

(19) United States
(12) Patent Application Publication
(10) Pub. No.: US 2026/0070924 A1
(43) Pub. Date: Mar. 12, 2026

(54) INDAZOLE MACROCYCLES AND THEIR USE

(71) Applicant: BLOSSOMHILL THERAPEUTICS, INC., San Diego, CA (US)

(72) Inventors: Jingrong Jean CUI, San Diego, CA (US); Eugene Yuanjin RUI, San Diego, CA (US); Evan W. ROGERS, San Diego, CA (US); Jane UNG, San Diego, CA (US); Ping JIANG, San Diego, CA (US); Dayong ZHAI, San Diego, CA (US); Wei DENG, San Diego, CA (US)

(21) Appl. No.: 18/871,700

(22) PCT Filed: Jun. 7, 2023
(86) PCT No.: PCT/US2023/068071

§ 371 Date: Dec. 4, 2024

Related U.S. Application Data
(60) Provisional application No. 63/350,307, filed on Jun. 8, 2022.
(60) Provisional application No. 63/501,114, filed on May 9, 2023.

(51) Int. Cl. C07D 498/18; A61K 31/4162; A61K 31/424; C07B 59/00; C07D 487/22; C07D 498/22

(52) U.S. Cl. CPC C07D 498/18; A61K 31/4162; A61K 31/424; C07B 59/002; C07D 487/22; C07D 498/22

(57) ABSTRACT

The present disclosure relates to indazole macrocyclic compounds, pharmaceutical compositions containing macrocyclic compounds, and methods of using macrocyclic compounds to treat disease, such as cancer comprising administering a therapeutically effective amount of a compound to a subject in need thereof.

### RELATED APPLICATIONS

This application claims the benefit of U.S. Provisional Application No. 63/350,307, filed Jun. 8, 2022, and U.S. Provisional Application No. 63/501,114, filed May 9, 2023, the entire disclosures of all of which are incorporated herein by reference.

### SEQUENCE LISTING

The application contains a Sequence Listing which has been submitted electronically in XML format and is hereby incorporated by reference in its entirety. Said XML copy, created on May 23, 2023, is named 83573-390331_SL.xml, and is 21,203 bytes in size.

### TECHNICAL FIELD

The present disclosure relates to indazole macrocyclic compounds, pharmaceutical compositions containing macrocyclic compounds, and methods of using macrocyclic compounds to treat disease, such as cancer.

### BACKGROUND

Protein kinases are tightly regulated signaling proteins that orchestrate the activation of signaling cascades by phosphorylating target proteins in response to extracellular and intricellular stimuli. The human genome encodes approximately 518 protein kinases (Manning G, et al The protein kinase complement of the human genome. Science. 2002, 298:1912-34). Dysregulation of kinase activity is associated with many diseases, including cancers, and cardiovascular, degenerative, immunological, infectious, inflammatory, and metabolic diseases (Levitzki, A. Protein kinase inhibitors as a therapeutic modality. Acc. Chem. Res. 2003, 36:462-469). The molecular bases leading to various diseases include kinase gain- and loss-of-function mutations, gene amplifications and deletions, splicing changes, and translocations (Wilson L J, et al New Perspectives, Opportunities, and Challenges in Exploring the Human Protein Kinome. Cancer Res. 2018, 78:15-29). The critical role of kinases in cancer and other diseases makes them attractive targets for drug inventions with 62 small molecule kinase inhibitors have been approved and 55 of them for cancer targeted therapies (Roskoski R Jr, Properties of FDA-approved Small Molecule Protein Kinase Inhibitors: A 2021 Update. Pharmacol Res 2021, 165:105463). Although kinase inhibitors have achieved dramatic success in cancer targeted therapies, the development of treatment resistance has remained as a challenge for small molecule kinase inhibitors. Acquired secondary mutations within kinase domain during the treatment often lead to treatment resistance to kinase inhibitors (Pottier C, et al Tyrosine Kinase Inhibitors in Cancer: Breakthrough and Challenges of Targeted Therapy. Cancers (Basel), 2020, 12:731). Resistance can also arise from subpopulations of tolerant/persister cells that survive in the presence of the treatment. Different processes contribute to the emergence of tolerant persister cells, including pathway rebound through the release of negative feedback loops, transcriptional rewiring mediated by chromatin remodeling and autocrine/paracrine communication among tumor cells and within the tumor microenvironment (Swayden M, et al Tolerant/Persister Cancer Cells and the Path to Resistance to Targeted Therapy. Cells 2020, 9, 2601). Therefore, it is necessary to invent kinase inhibitors that can target not only the kinase oncogenic drivers, overcome most frequent resistance mutations, but also tolerant persister cancer cells for overcoming resistance, achieving better efficacy and longer disease control.

Acute myeloid leukemia (AML) is a complex malignancy with many cytogenetic or chromosomal aberrations. The most frequently identified mutation in AML is FMS-like tyrosine kinase 3 (FLT3) with about 25% of adult patients having FLT3 internal tandem duplication (FLT3-ITD) and 7-10% with point mutations or deletions (Daver N, et al Targeting FLT3 mutations in AML: review of current knowledge and evidence. Leukemia 2019, 33:299-312). Two FLT3 inhibitors have been approved by the Food and Drug Administration (FDA) for AML indications: midostaurin for newly diagnosed FLT3 mutated AML in combination with standard induction and consolidation chemotherapy and gilteritinib for relapsed or refractory FLT3 mutated AML as monotherapy. Although significant progress has been made in the treatment of AML with FLT3 inhibitors, leukemia relapse remains to be a major cause of treatment failure. Mechanisms of drug resistance include evolution of FLT3 resistance mutations, adaptive cellular mechanisms, and a protective leukemia microenvironment. Frequently recurrent locations for resistance mutations are in the activating loop residues (e.g., D835, I836, D839, and Y842) or in the gatekeeper residue F691 of FLT3. Alterations of the leukemia microenvironment, including increased FGF2 and CXCL12/CXCR4 signaling, may protect FLT3-mutated progenitors. Increased signaling through parallel prosurvival pathways, including RAS-RAF-MEK-ERK, PI3K-AKT-mTOR, and JAK-STAT5-PIM1 pathways may also contribute to FLT3 inhibitor resistance (Short N J, et al Advances in the Treatment of Acute Myeloid Leukemia: New Drugs and New Challenges. Cancer Discov. 2020, 10:506-525).

The proviral integration for the Moloney murine leukemia virus (PIM) kinases are oncogenic serine/threonine kinases that phosphorylate a wide range of substrates that regulate several of the hallmarks of cancer including tumor metabolism, survival, metastasis, immune evasion and inflammation (Toth R K, Warfel N A. Targeting PIM Kinases to Overcome Therapeutic Resistance in Cancer. Mol Cancer Ther. 2021, 20(1):3-10). PIM kinases interact with numerous major oncogenic players, including stabilization of p53, synergism with c-Myc, and notable parallel signaling with PI3K/Akt. The aberrant PIM kinase activity plays an important role in resistance mechanisms of chemotherapy, radiotherapy, anti-angiogenic therapies and targeted therapies, providing a rationale for co-targeting treatment strategies for a more durable patient response (Malone T, et al Current perspectives on targeting PIM kinases to overcome mechanisms of drug resistance and immune evasion in cancer. Pharmacol Ther 2020 March; 207).

Cdc-like kinases (CLKs) are evolutionary conserved dual-specificity kinases that are able to phosphorylate serine, threonine, and tyrosine residues. CLKs catalyze the phosphorylation of SR proteins, serine, and arginine-rich splicing factors 1-12 (SRSF1-12), which regulate the spliceosome molecular machinery (Martín Moyano P, et al Cdc-Like Kinases (CLKs): Biology, Chemical Probes, and Therapeutic Potential. Int J Mol Sci 2020, 21(20):7549). Dysregulation of alternative splicing is a feature of cancer. High-frequency mutations of SF3B1 or SRSF2 have been described in patients with myelodysplastic syndromes (MDS), chronic myelomonocytic leukemia, and acute myeloid leukemia (AML) (Papaemmanuil et al, Genomic classification and prognosis in acute myeloid leukemia. N Engl J Med. 2016, 374:2209-2221). In addition, mutations in splicing-related genes have also been found in various solid cancers, including lung, breast, and pancreatic cancers (Dvinge H, et al RNA splicing factors as oncoproteins and tumour suppressors. Nat Rev Cancer 2016, 16: 413-430). The modulation of pre-mRNA splicing via inhibition of CLK kinases is an attractive anti-neoplastic strategy, especially for the cancers that exhibit aberrant pre-mRNA splicing.

Therefore, it is necessary to develop a new generation multitargeted FLT3 inhibitors that are potent against oncogenic driver FLT3 mutations, other emerging and established FLT3 resistance mutations, as well as emerging resistance targets for tolerant/persistent cancer cells, e.g., PIM kinases and CLK kinases.

### SUMMARY

In one aspect, the disclosure provides a compound of the formula I, or a pharmaceutically acceptable salt thereof,

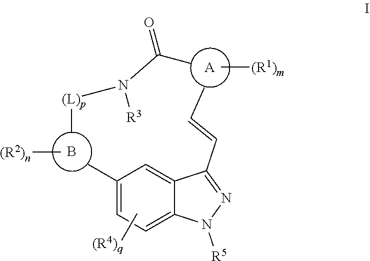

wherein R¹, R², R³, R⁴, R⁵, A, B, L, m, n, p, and q are as described herein.

In some embodiments, the disclosure provides a compound of the formula II, or a pharmaceutically acceptable salt thereof,

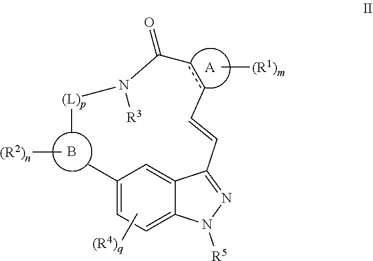

wherein R¹, R², R³, R⁴, R⁵, A, B, L, m, n, p, q, and “ [symbol] ” are as described herein.

In some embodiments, the disclosure provides a compound of the formula III, or a pharmaceutically acceptable salt thereof,

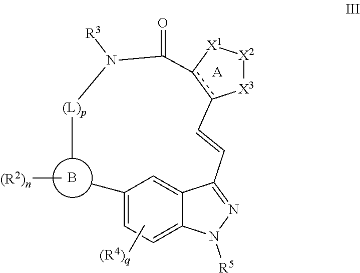

wherein R¹, R², R³, R⁴, R⁵, A, B, L, X¹, X², X³, m, n, p, q, and “ [symbol] ” are as described herein.

In further aspects, the disclosure relates to a pharmaceutical composition comprising at least one compound of Formula (I)-(VI) or a pharmaceutically acceptable salt thereof. Pharmaceutical compositions according to the disclosure may further comprise a pharmaceutically acceptable excipient.

In further aspects, the disclosure relates to a compound of Formula (I)-(VI), or a pharmaceutically acceptable salt thereof, for use as a medicament.

In further aspects, the disclosure relates to a method of treating disease, such as cancer comprising administering to a subject in need of such treatment an effective amount of at least one compound of Formula (I)-(VI), or a pharmaceutically acceptable salt thereof.

In further aspects, the disclosure relates to use of a compound of Formula (I)-(VI), or a pharmaceutically acceptable salt thereof, in the preparation of a medicament for the treatment of disease, such as cancer, and the use of such compounds and salts for treatment of such diseases.

In further aspects, the disclosure relates to a method of inhibiting one or more of aberrant FLT3, including oncogenic driver mutations such as FLT3-ITD and FLT3 resistance mutations, such as resistance mutations in the activating loop residues (e.g., D835, I836, D839, and Y842), or in the gatekeeper residue F691 of FLT3, aberrant PIM kinases, and/or aberrant CLK kinases comprising contacting a cell comprising one or more of aberrant FLT3, including oncogenic driver mutations such as FLT3-ITD and FLT3 resistance mutations, such as resistance mutations in the activating loop residues (e.g., D835, 1836, D839, and Y842), or in the gatekeeper residue F691 of FLT3, aberrant PIM kinases, and/or aberrant CLK kinases. with an effective amount of at least one compound of Formula (I)-(VI), or a pharmaceutically acceptable salt thereof, and/or with at least one pharmaceutical composition of the disclosure, wherein the contacting is in vitro, ex vivo, or in vivo.

Additional embodiments, features, and advantages of the disclosure will be apparent from the following detailed description and through practice of the disclosure. The compounds of the present disclosure can be described as embodiments in any of the following enumerated clauses. It will be understood that any of the embodiments described herein can be used in connection with any other embodiments described herein to the extent that the embodiments do not contradict one another.

1. A compound of the formula I

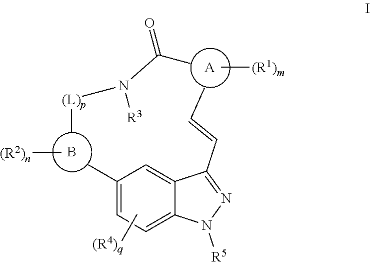

wherein
   ring A is a 5- to 10-membered heteroarylene;
   ring B is a 5- to 10-membered heteroarylene or C₆-C₁₀ arylene;
   each L is independently —O—, —S—, —S(O)—, —S(O)₂—, —N(R⁶)C(O)—, —C(O)N(R⁶)—, —N(R⁶)—, —N(R⁶)S(O)—, —S(O)N(R⁶)—, —N(R⁶)S(O)₂—, —S(O)₂N(R⁶)—, or —C(R⁷)(R⁸)—, provided that (L)ₚ does not comprise an O—O, S—O, or N—N bond, and the point of covalent attachment of (L)ₚ to —NR³— does not form a —N—N— or a —O—N— bond;
   each R¹ and R² when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{c}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
R³ is H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NRCS(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁴ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁵ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR′;

each R⁶, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;

each R⁷ and R⁸, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R⁷ and R⁸, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁷ and R⁸ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R¹ is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{c} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₇-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, 3, or 4;
p is 3, 4, 5, 6, or 7; and
q is 0, 1, or 2
or a pharmaceutically acceptable salt thereof.

2. A compound of the formula I

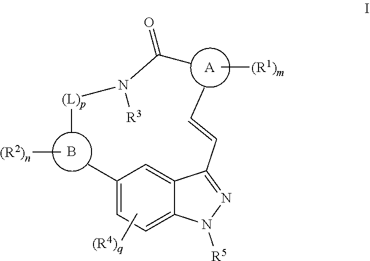

wherein
ring A is a 5- to 10-membered heteroarylene;
ring B is a 5- to 10-membered heteroarylene or C₆-C₁₀ arylene;
each L is independently —O—, —S—, —S(O)—, —S(O)₂—, —N(R⁶)C(O)—, —C(O)N(R⁶)—, —N(R⁶)—, —N(R⁶)S(O)—, —S(O)N(R⁶)—, —N(R⁶)S(O)₂—, —S(O)₂N(R⁶)—, or —C(R⁷)(R⁸)—, provided that (L)ₚ does not comprise an O—O, S—O, or N—N bond, and the point of covalent attachment of (L)ₚ to —NR³— does not form a —N—N— or a —O—N— bond;

each R¹ and R² when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR'S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
R³ is H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{C}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR'S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁴ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁵ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR^{c};
each R⁶, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{e}, —S(O)₂R^{e}, —S(O)NR^{c}R^{d}, —S(O)₂NR^{c}R^{d}, —NR^{c}R^{d}, —NR^{c}C(O)R^{d}, —N(C(O)R^{e})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁷ and R⁸, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{c}, —C(O)NR^{c}R^{f}, —PR^{c}R^{f}, —P(O)R^{c}R^{f}, —P(O)₂R^{c}R^{f}, —P(O)NR^{c}R^{f}, —P(O)₂NR^{c}R^{f}, —P(O)OR^{e}, —P(O)₂OR^{e}, —CN, or —NO₂; or two of R⁷ and R⁸, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁷ and R⁸ are taken together is independently optionally substituted by —OR^{c}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;

each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{e} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, 3, or 4;
p is 3, 4, 5, 6, or 7; and
q is 0, 1, or 2
or a pharmaceutically acceptable salt thereof.

3. The compound of clause 1 or 2, or a pharmaceutically acceptable salt thereof, having the formula II

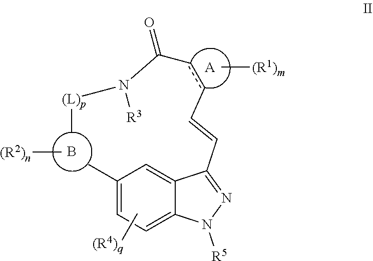

wherein “ [symbol] ” is optionally a carbon-carbon single bond or a carbon-carbon double bond, and ring A is a 5-membered heteroarylene.

4. The compound of clause 1, 2, or 3, or a pharmaceutically acceptable salt thereof, having the formula III

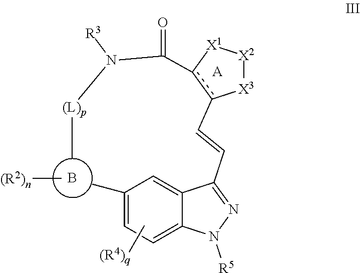

wherein
X¹, X², and X³ are each independently —O—, —S—, ═C(H)—, ═C(R¹)—, —N(H)—, —N(R¹)— or ═N— and ring A is a 5-membered heteroarylene, provided that at least one of X¹, X², and X³ is not ═C(H)—, or ═C(R¹)—; and
“ [symbol] ” is optionally a carbon-carbon single bond or a carbon-carbon double bond.

5. The compound of any one of the preceding clauses, or a pharmaceutically acceptable salt thereof, wherein ring A is a 5-membered heteroarylene selected from the group consisting of

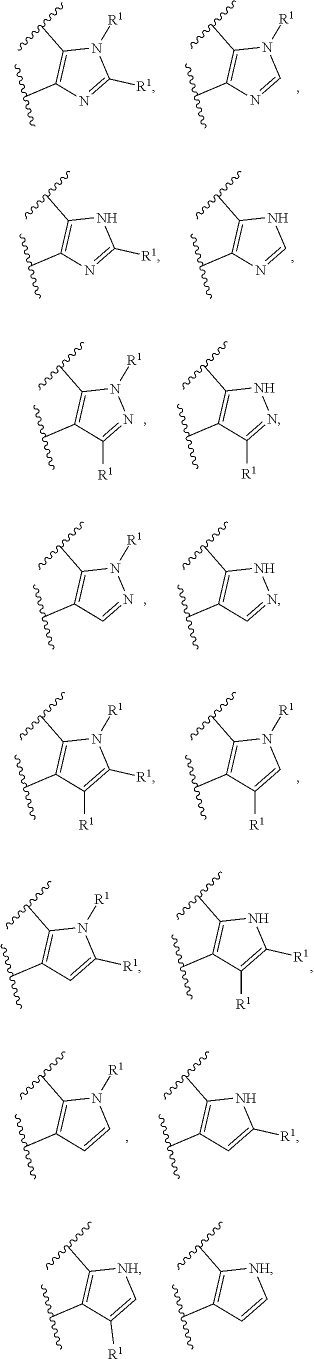

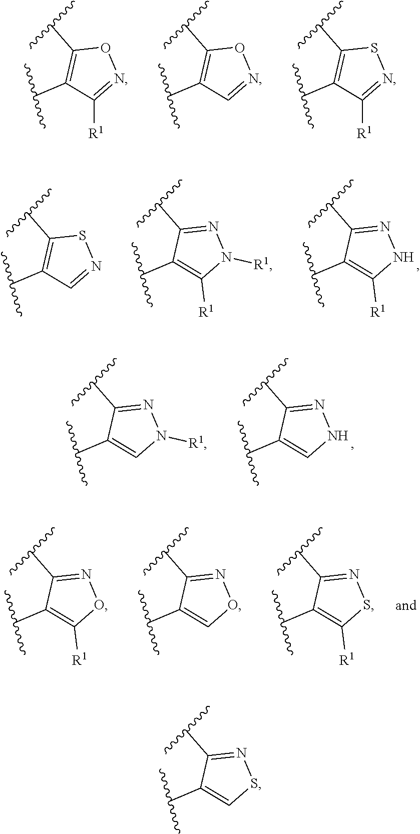

wherein each “ [symbol] ” represents a point of covalent attachment.

6. The compound of any one of the preceding clauses, or a pharmaceutically acceptable salt thereof, wherein ring A is a 5-membered heteroarylene selected from the group consisting of

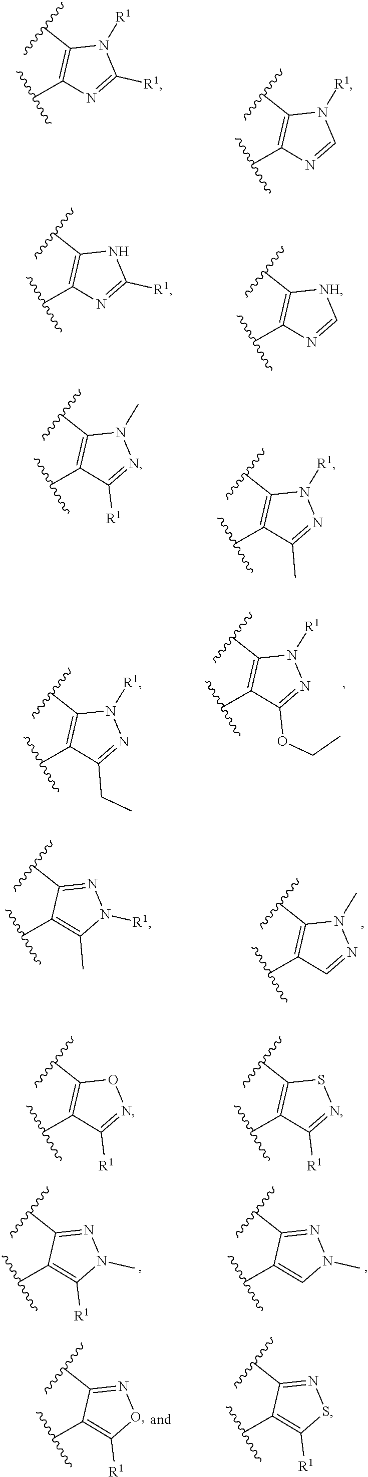

wherein each “ [symbol] ” represents a point of covalent attachment.

7. The compound of any one of the preceding clauses, or a pharmaceutically acceptable salt thereof, wherein ring A is a 5-membered heteroarylene selected from the group consisting of

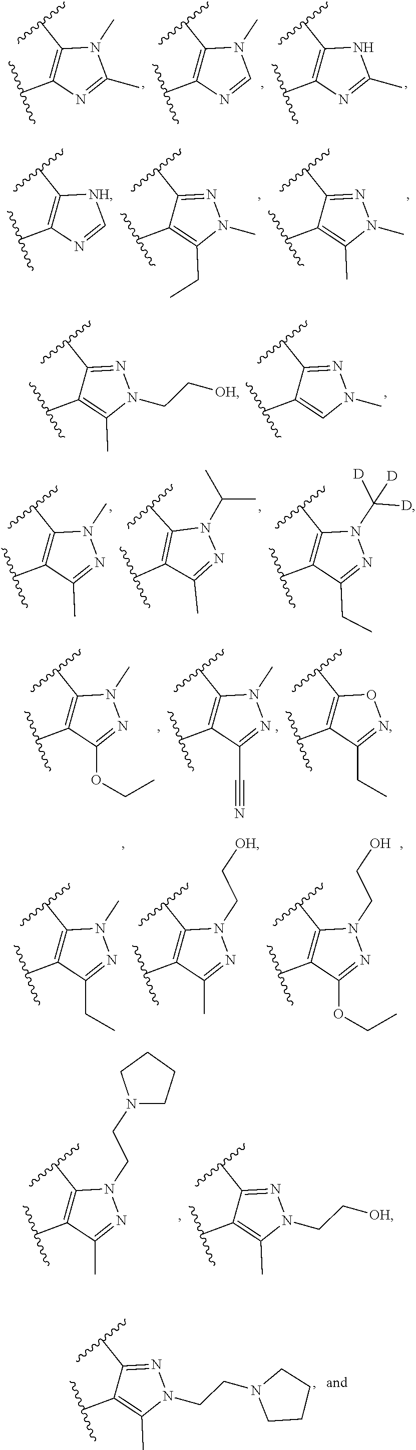

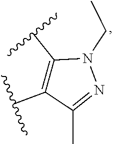 [SMILES: *c1c(C)nn(CC)c1*]

wherein each “ [symbol] ” represents a point of covalent attachment.

8. The compound of any one of the preceding clauses, or a pharmaceutically acceptable salt thereof, wherein ring B is a C₆-C₁₀ arylene, and n is 0, 1, or 2.

9. The compound of any one of the preceding clauses, or a pharmaceutically acceptable salt thereof, wherein ring B is a phenylene, and n is 0, 1, or 2.

10. The compound of any one of the preceding clauses, or a pharmaceutically acceptable salt thereof, wherein ring B is a phenylene, and n is 0 or 1.

11. The compound of any one of the preceding clauses, or a pharmaceutically acceptable salt thereof, wherein ring B is a phenylene, n is 1, and R² is methyl, ethyl, F, Cl, or Br.

12. The compound of any one of the preceding clauses, or a pharmaceutically acceptable salt thereof, wherein ring B is

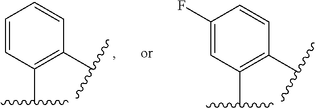 [SMILES: *c1ccccc1* ; *c1ccc(F)cc1*]

wherein each “ [symbol] ” represents a point of covalent attachment.

13. The compound of any one of clauses 1 to 7, or a pharmaceutically acceptable salt thereof, wherein ring B is a 5- to 10-membered heteroarylene.

14. The compound of any one of clauses 1 to 7 or 13, or a pharmaceutically acceptable salt thereof, wherein ring B is a 5-membered heteroarylene selected from the group consisting of

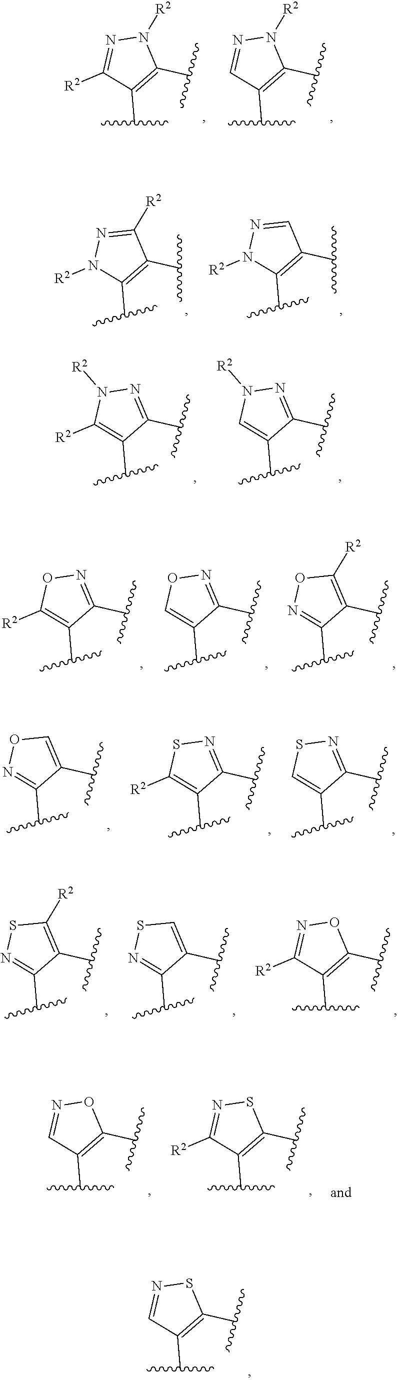

wherein each “ [symbol] ” represents a point of covalent attachment.

15. The compound of any one of clauses 1 to 7, 13, or 14, or a pharmaceutically acceptable salt thereof, wherein ring B is a 5-membered heteroarylene selected from the group consisting of

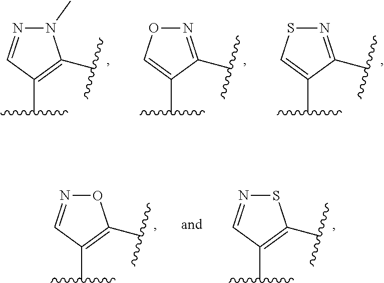

wherein each “ [symbol] ” represents a point of covalent attachment.

16. The compound of any one of the preceding clauses, or a pharmaceutically acceptable salt thereof, wherein R³ is H or methyl.

17. The compound of any one of the preceding clauses, or a pharmaceutically acceptable salt thereof, wherein R⁴ is H or methyl.

18. The compound of any one of the preceding clauses, or a pharmaceutically acceptable salt thereof, wherein R⁵ is H.

19. The compound of any one of the preceding clauses, or a pharmaceutically acceptable salt thereof, wherein each L is independently each L is independently —C(R⁷)(R⁸)—, —C(O)—, —O—, or —N(R⁶)—, provided that (L)ₚ does not comprise a —O—O— or a —O—N(R⁶)— bond, and the point of covalent attachment of (L)ₚ to —NR³— does not form a —N—N— or a —O—N-bond.

20. The compound of any one of the preceding clauses, or a pharmaceutically acceptable salt thereof, wherein -(L)ₚ- is —(CR⁷R⁸)C(O)N(R⁶)—(CR⁷R⁸)₂—, —(CR⁷R⁸)N(R⁶)C(O)—(CR⁷R⁸)₂—, —N(R⁶)—C(O)(CR⁷R⁸)₂O(CR⁷R⁸)₂—, —CR⁷R⁸O(CR⁷R⁸)₂O—(CR⁷R⁸)₂, —O(CR⁷R⁸)₂O(CR⁷R⁸)₂—, —CR⁷R⁸O—CR⁷R⁸—C(O)N(R⁶)—(CR⁷R⁸)₂—, —(CR⁷R⁸)₃O(CR⁷R⁸)₂—, —(CR⁷R⁸)₂O(CR⁷R⁸)₃—, —CR⁷R⁸—N(R⁶)—(CR⁷R⁸)₂—, —CR⁷R⁸—N(R⁶)—(CR⁷R⁸)₃—, —O(CR⁷R⁸)₂O(CR⁷R⁸)₃—, —(CR⁷R⁸)₂—N(R⁶)—(CR⁷R⁸)₃—, —(CR⁷R⁸)₂—N(R⁶)—(CR⁷R⁸)₂—, —O—(CR⁷R⁸)₂—, —O—(CR⁷R⁸)₃—, or —O—(CR⁷R⁸)₄—.

21. The compound of any one of the preceding clauses, or a pharmaceutically acceptable salt thereof, wherein R⁶ is H or methyl.

22. The compound of any one of the preceding clauses, or a pharmaceutically acceptable salt thereof, wherein each R⁷ and R¹ is H.

23. The compound of any one of the preceding clauses, or a pharmaceutically acceptable salt thereof, wherein -(L)ₚ- is —CH₂N(H)—(CH₂)₂—, —CH₂N(CH₃)—(CH₂)₂—, —O(CH₂)₂—, —OCH(CH₃)CH₂—, —O(CH₂)₃—, —O(CH₂)₄—, and —O(CH₂)₂O(CH₂)₂—.

24. The compound of clause 1, selected from the group consisting of

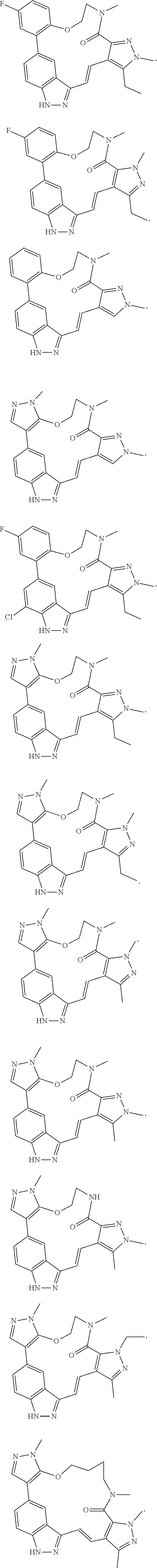

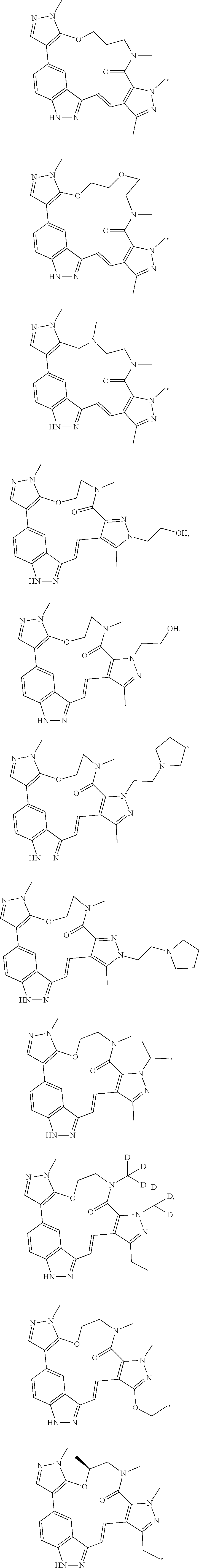

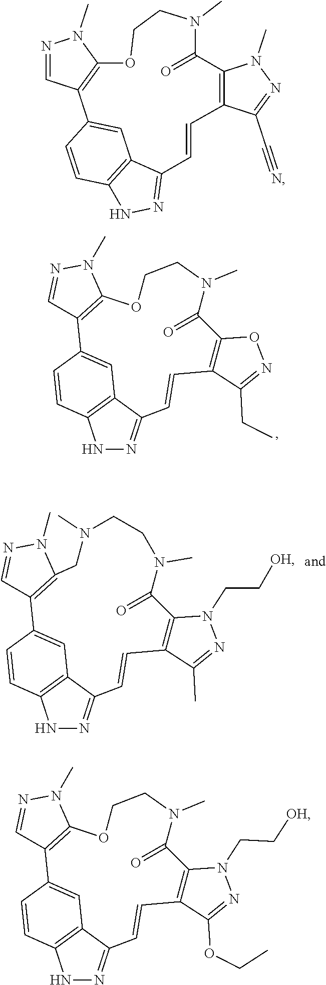

or a pharmaceutically acceptable salt thereof.

25. A pharmaceutical composition comprising a compound of any one of the preceding clauses, and optionally one or more excipients.

26. A method of treating disease in a subject comprising, administering a therapeutically effective amount of a compound of any one of clauses 1 to 24, or a pharmaceutical composition of clause 25.

27. A compound according to any one of clauses 1 to 24, for use in a method of treating disease in a subject.

28. Use of a compound according to any one of clauses 1 to 24 in the manufacture of a medicament for the treatment of disease in a subject.

### DETAILED DESCRIPTION

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

For the sake of brevity, the disclosures of the publications cited in this specification, including patents, are herein incorporated by reference. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference.

As used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, Fourth Edition, New York: Oxford University Press, 2002, pp. 360-361, 1084-1085; Smith and March, March's Advanced Organic Chemistry: Reactions, Mechanisms, and Structure, Fifth Edition, Wiley-Interscience, 2001.

Chemical nomenclature for compounds described herein has generally been derived using the commercially-available ACD/Name 2014 (ACD/Labs) or ChemBioDraw Ultra 13.0 (Perkin Elmer).

As used herein and in connection with chemical structures depicting the various embodiments described herein, “*”, “**”, and “ [symbol] ”, each represent a point of covalent attachment of the chemical group or chemical structure in which the identifier is shown to an adjacent chemical group or chemical structure. For example, in a hypothetical chemical structure A-B, where A and B are joined by a covalent bond, in some embodiments, the portion of A-B defined by the group or chemical structure A can be represented by “A-*”, “A-**”, or

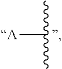

where each of “-*”, “-**”, and

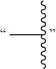

represents a bond to A and the point of covalent bond attachment to B. Alternatively, in some embodiments, the portion of A-B defined by the group or chemical structure B can be represented by “*—B”, “**—B”, or

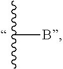

where each of “-*”, “**-”, and

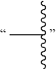

represents a bond to B and the point of covalent bond attachment to A.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Chemical Definitions

The term “alkyl” refers to a straight- or branched-chain monovalent hydrocarbon group. The term “alkylene” refers to a straight- or branched-chain divalent hydrocarbon group. In some embodiments, it can be advantageous to limit the number of atoms in an “alkyl” or “alkylene” to a specific range of atoms, such as C₁-C₂₀ alkyl or C₁-C₂₀ alkylene, C₁-C₁₂ alkyl or C₁-C₁₂ alkylene, or C₁-C₆ alkyl or C₁-C₆ alkylene. Examples of alkyl groups include methyl (Me), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. Examples of alkylene groups include methylene (—CH₂—), ethylene ((—CH₂—)₂), n-propylene ((—CH₂—)₃), iso-propylene ((—C(H)(CH₃)CH₂—)), n-butylene ((—CH₂—)₄), and the like. It will be appreciated that an alkyl or alkylene group can be unsubstituted or substituted as described herein. An alkyl or alkylene group can be substituted with any of the substituents in the various embodiments described herein, including one or more of such substituents.

The term “alkenyl” refers to a straight- or branched-chain mono-valent hydrocarbon group having one or more double bonds. The term “alkenylene” refers to a straight- or branched-chain di-valent hydrocarbon group having one or more double bonds. In some embodiments, it can be advantageous to limit the number of atoms in an “alkenyl” or “alkenylene” to a specific range of atoms, such as C₂-C₂₀ alkenyl or C₂-C₂₀ alkenylene, C₂-C₁₂ alkenyl or C₂-C₁₂ alkenylene, or C₂-C₆ alkenyl or C₂-C₆ alkenylene. Examples of alkenyl groups include ethenyl (or vinyl), allyl, and but-3-en-1-yl. Examples of alkenylene groups include ethenylene (or vinylene) (—CH═CH—), n-propenylene (—CH═CHCH₂—), iso-propenylene (—CH═CH(CH₃)—), and the like. Included within this term are cis and trans isomers and mixtures thereof. It will be appreciated that an alkenyl or alkenylene group can be unsubstituted or substituted as described herein. An alkenyl or alkenylene group can be substituted with any of the substituents in the various embodiments described herein, including one or more of such substituents.

The term “alkynyl” refers to a straight- or branched-chain monovalent hydrocarbon group having one or more triple bonds. The term “alkynylene” refers to a straight- or branched-chain divalent hydrocarbon group having one or more triple bonds. In some embodiments, it can be advantageous to limit the number of atoms in an “alkynyl” or “alkynylene” to a specific range of atoms, such as C₂-C₂₀ alkynyl or C₂-C₂₀ alkynylene, C₂-C₁₂ alkynyl or C₂-C₁₂ alkynylene, or C₂-C₆ alkynyl or C₂-C₆ alkynylene. Examples of alkynyl groups include acetylenyl (—C≡CH) and propargyl (—CH₂C≡CH), but-3-yn-1,4-diyl (—C≡C—CH₂CH₂—), and the like. It will be appreciated that an alkynyl or alkynylene group can be unsubstituted or substituted as described herein. An alkynyl or alkynylene group can be substituted with any of the substituents in the various embodiments described herein, including one or more of such substituents.

The term “cycloalkyl” refers to a saturated or partially saturated, monocyclic or polycyclic mono-valent carbocycle. The term “cycloalkylene” refers to a saturated or partially saturated, monocyclic or polycyclic divalent carbocycle. In some embodiments, it can be advantageous to limit the number of atoms in a “cycloalkyl” or “cycloalkylene” to a specific range of atoms, such as having 3 to 12 ring atoms. Polycyclic carbocycles include fused, bridged, and spiro polycyclic systems. Illustrative examples of cycloalkyl groups include monovalent radicals of the following entities, while cycloalkylene groups include divalent radicals of the following entities, in the form of properly bonded moieties:

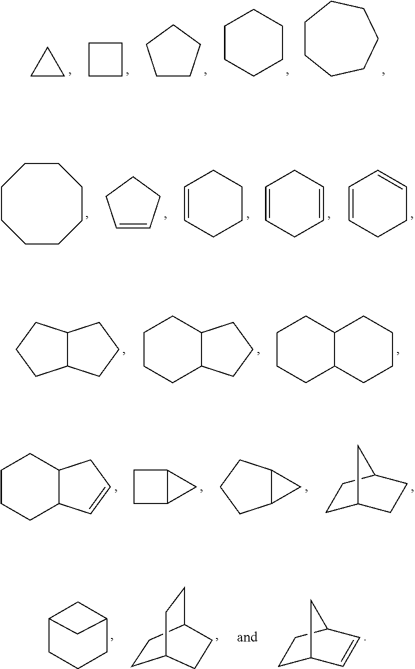

In particular, a cyclopropyl moiety can be depicted by the structural formula

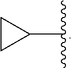 [SMILES: *C1CC1]

### In

particular, a cyclopropylene moiety can be depicted by the structural formula

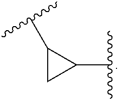 [SMILES: *C1CC1*]

It will be appreciated that a cycloalkyl or cycloalkylene group can be unsubstituted or substituted as described herein. A cycloalkyl or cycloalkylene group can be substituted with any of the substituents in the various embodiments described herein, including one or more of such substituents.

The term “halogen” or “halo” represents chlorine, fluorine, bromine, or iodine.

The term “haloalkyl” refers to an alkyl group with one or more halo substituents. Examples of haloalkyl groups include —CF₃, —(CH₂)F, —CHF₂, —CH₂Br, —CH₂CF₃, and —CH₂CH₂F. The term “haloalkylene” refers to an alkyl group with one or more halo substituents. Examples of haloalkyl groups include —CF₂—, —C(H)(F)—, —C(H)(Br)—, —CH₂CF₂—, and —CH₂C(H)(F)—.

The term “aryl” refers to a monovalent all-carbon monocyclic or fused-ring polycyclic group having a completely conjugated pi-electron system. The term “arylene” refers to a divalent all-carbon monocyclic or fused-ring polycyclic group having a completely conjugated pi-electron system. In some embodiments, it can be advantageous to limit the number of atoms in an “aryl” or “arylene” to a specific range of atoms, such as mono-valent all-carbon monocyclic or fused-ring polycyclic groups of 6 to 14 carbon atoms (C₆-C₁₄ aryl), monovalent all-carbon monocyclic or fused-ring polycyclic groups of 6 to 10 carbon atoms (C₆-C₁₀ aryl), divalent all-carbon monocyclic or fused-ring polycyclic groups of 6 to 14 carbon atoms (C₆-C₁₄ arylene), divalent all-carbon monocyclic or fused-ring polycyclic groups of 6 to 10 carbon atoms (C₆-C₁₀ arylene). Examples, without limitation, of aryl groups are phenyl, naphthalenyl and anthracenyl. Examples, without limitation, of arylene groups are phenylene, naphthalenylene and anthracenylene. It will be appreciated that an aryl or arylene group can be unsubstituted or substituted as described herein. An aryl or arylene group can be substituted with any of the substituents in the various embodiments described herein, including one or more of such substituents.

The term “heterocycloalkyl” refers to a mono-valent monocyclic or polycyclic ring structure that is saturated or partially saturated having one or more non-carbon ring atoms. The term “heterocycloalkylene” refers to a divalent monocyclic or polycyclic ring structure that is saturated or partially saturated having one or more non-carbon ring atoms. In some embodiments, it can be advantageous to limit the number of atoms in a “heterocycloalkyl” or “heterocycloalkylene” to a specific range of ring atoms, such as from 3 to 12 ring atoms (3- to 12-membered), or 3 to 7 ring atoms (3- to 7-membered), or 3 to 6 ring atoms (3- to 6-membered), or 4 to 6 ring atoms (4- to 6-membered), 5 to 7 ring atoms (5- to 7-membered), or 4 to 10 ring atoms (4- to 10-membered). In some embodiments, it can be advantageous to limit the number and type of ring heteroatoms in “heterocycloalkyl” or “heterocycloalkylene” to a specific range or type of heteroatoms, such as 1 to 5 ring heteroatoms selected from nitrogen, oxygen, and sulfur. Polycyclic ring systems include fused, bridged, and spiro systems. The ring structure may optionally contain an oxo group or an imino group on a carbon ring member or up to two oxo groups on sulfur ring members. Illustrative examples of heterocycloalkyl groups include monovalent radicals of the following entities, while heterocycloalkylene groups include divalent radicals of the following entities, in the form of properly bonded moieties:

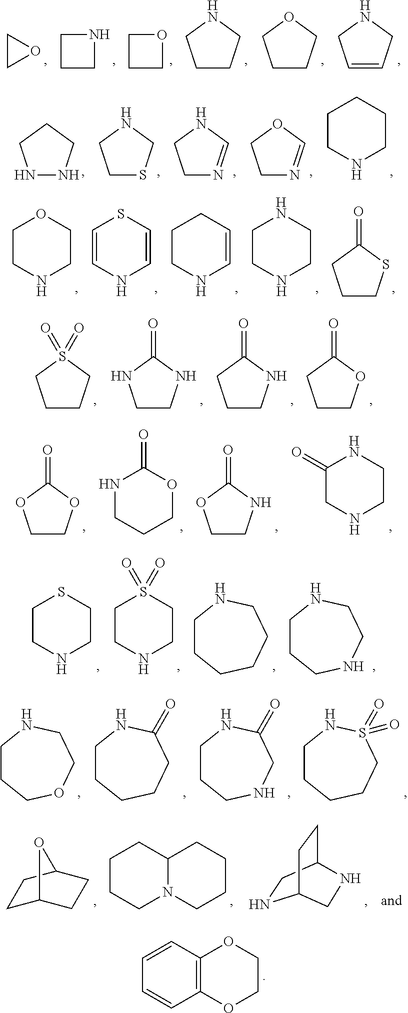

A three-membered heterocycle may contain at least one heteroatom ring atom, where the heteroatom ring atom is a sulfur, oxygen, or nitrogen. Non-limiting examples of three-membered heterocycle groups include monovalent and divalent radicals of oxirane, azetidine, and thiirane. A four-membered heterocycle may contain at least one heteroatom ring atom, where the heteroatom ring atom is a sulfur, oxygen, or nitrogen. Non-limiting examples of four-membered heterocycle groups include monovalent and divalent radicals of azitidine, oxtenane, and thietane. A five-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heterocyle groups include mono-valent and divalent radicals of pyrrolidine, tetrahydrofuran, 2, 5-dihydro-1H-pyrrole, pyrazolidine, thiazolidine, 4,5-dihydro-1H-imidazole, dihydrothiophen-2(3H)-one, tetrahydrothiophene 1,1-dioxide, imidazolidin-2-one, pyrrolidin-2-one, dihydrofuran-2(3H)-one, 1,3-dioxolan-2-one, and oxazolidin-2-one. A six-membered heterocycle can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heterocycle groups include mono-valent or divalent radicals of piperidine, morpholine, 4H-1,4-thiazine, 1,2,3,4-tetrahydropyridine, piperazine, 1,3-oxazinan-2-one, piperazin-2-one, thiomorpholine, and thiomorpholine 1,1-dioxide. A “heterobicycle” is a fused bicyclic system comprising one heterocycle ring fused to a cycloalkyl or another heterocycle ring.

It will be appreciated that a heterocycloalkyl or heterocycloalkylene group can be unsubstituted or substituted as described herein. A heterocycloalkyl or heterocycloalkylene group can be substituted with any of the substituents in the various embodiments described herein, including one or more of such substituents.

The term “heteroaryl” refers to a mono-valent monocyclic, fused bicyclic, or fused polycyclic aromatic heterocycle (ring structure having ring atoms or members selected from carbon atoms and up to four heteroatoms selected from nitrogen, oxygen, and sulfur) that is fully unsaturated and having from 3 to 12 ring atoms per heterocycle. The term “heteroarylene” refers to a divalent monocyclic, fused bicyclic, or fused polycyclic aromatic heterocycle (ring structure having ring atoms or members selected from carbon atoms and up to four heteroatoms selected from nitrogen, oxygen, and sulfur) having from 3 to 12 ring atoms per heterocycle. In some embodiments, it can be advantageous to limit the number of ring atoms in a “heteroaryl” or “heteroarylene” to a specific range of atom members, such as 5- to 10-membered heteroaryl or 5- to 10-membered heteroarylene. In some instances, a 5- to 10-membered heteroaryl can be a monocyclic ring or fused bicyclic rings having 5- to 10-ring atoms wherein at least one ring atom is a heteroatom, such as N, O, or S. In some instances, a 5- to 10-membered heteroarylene can be a monocyclic ring or fused bicyclic rings having 5- to 10-ring atoms wherein at least one ring atom is a heteroatom, such as N, O, or S. The ring structure may optionally contain an oxo group or an imino group on a carbon ring member or up to two oxo groups on sulfur ring members. Illustrative examples of 5- to 10-membered heteroaryl groups include monovalent radicals of the following entities, while examples of 5- to 10-membered heteroarylene groups include divalent radicals of the following entities, in the form of properly bonded moieties:

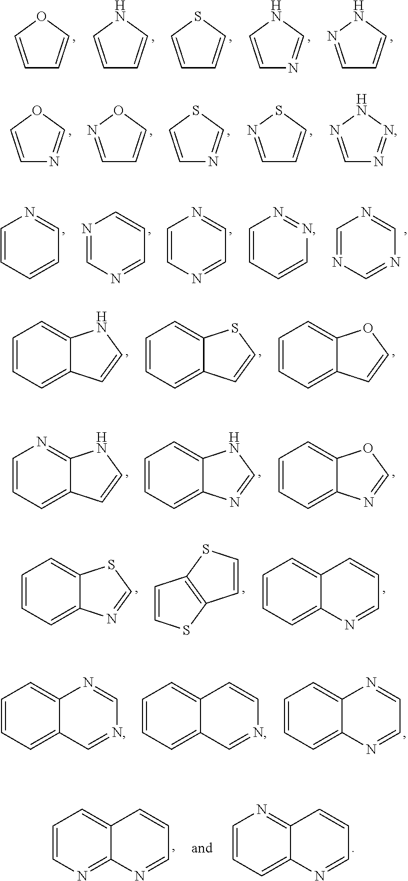

In some embodiments, a “monocyclic” heteroaryl can be an aromatic five- or six-membered heterocycle. A five-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of five-membered heteroaryl groups include mono-valent radicals of furan, thiophene, pyrrole, oxazole, isoxazole, thiazole, isothiazole, pyrazole, imidazole, oxadiazole, thiadiazole, triazole, or tetrazole. Non-limiting examples of five-membered heteroarylene groups include di-valent radicals of furan, thiophene, pyrrole, oxazole, isoxazole, thiazole, isothiazole, pyrazole, imidazole, oxadiazole, thiadiazole, triazole, or tetrazole. A six-membered heteroaryl or heteroarylene can contain up to four heteroatom ring atoms, where (a) at least one ring atom is oxygen and sulfur and zero, one, two, or three ring atoms are nitrogen, or (b) zero ring atoms are oxygen or sulfur and up to four ring atoms are nitrogen. Non-limiting examples of six-membered heteroaryl groups include monovalent radicals of pyridine, pyrazine, pyrimidine, pyridazine, or triazine. Non-limiting examples of six-membered heteroarylene groups include divalent radicals of pyridine, pyrazine, pyrimidine, pyridazine, or triazine. A “bicyclic heteroaryl” or “bicyclic heteroarylene” is a fused bicyclic system comprising one heteroaryl ring fused to a phenyl or another heteroaryl ring. Non-limiting examples of bicyclic heteroaryl groups include monovalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole. Non-limiting examples of bicyclic heteroarylene groups include divalent radicals of quinoline, isoquinoline, quinazoline, quinoxaline, 1,5-naphthyridine, 1,8-naphthyridine, isoquinolin-3(2H)-one, thieno[3,2-b]thiophene, 1H-pyrrolo[2,3-b]pyridine, 1H-benzo[d]imidazole, benzo[d]oxazole, and benzo[d]thiazole.

In particular, a pyrazolyl moiety can be depicted by the structural formula

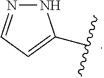 [SMILES: *c1ccn[nH]1]

In particular, an example of a pyrazolylene moiety can be depicted by the structural formula

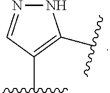 [SMILES: *c1cn[nH]c1*]

It will be appreciated that a heteroaryl or heteroarylene group can be unsubstituted or substituted as described herein. A heteroaryl or heteroarylene group can be substituted with any of the substituents in the various embodiments described herein, including one or more of such substituents.

The term “oxo” represents a carbonyl oxygen. For example, a cyclopentyl substituted with oxo is cyclopentanone.

The term “substituted” means that the specified group or moiety bears one or more substituents. The term “unsubstituted” means that the specified group bears no substituents. Where the term “substituted” is used to describe a structural system, the substitution is meant to occur at any valency-allowed position on the system. In some embodiments, “substituted” means that the specified group or moiety bears one, two, or three substituents. In other embodiments, “substituted” means that the specified group or moiety bears one or two substituents. In still other embodiments, “substituted” means the specified group or moiety bears one substituent.

Any formula depicted herein is intended to represent a compound of that structural formula as well as certain variations or forms. For example, a formula given herein is intended to include a racemic form, or one or more enantiomeric, diastereomeric, or geometric isomers, or a mixture thereof. Additionally, any formula given herein is intended to refer also to a hydrate, solvate, or polymorph of such a compound, or a mixture thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labelled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Certain chemical entities of Formula (I)-(VI) may be depicted in two or more tautomeric forms. Any and all alternative tautomers are included within the scope of these formulas, and no inference should be made as to whether the chemical entity exists as the tautomeric form in which it is drawn. It will be understood that certain chemical entities described herein can exist in different tautomeric forms. It will be readily appreciated by one of skill in the art that because of rapid interconversion, tautomers can generally be considered to be the same chemical compound. Examples of tautomers include but are not limited to enol-keto tautomers, amine-imine tautomers, and the like.

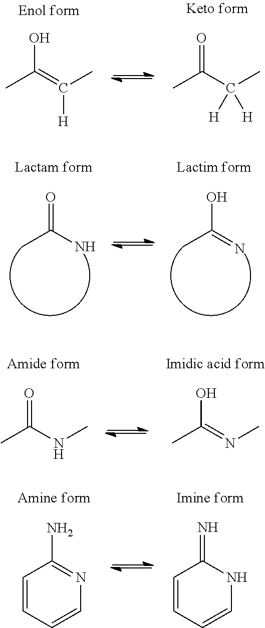

The nomenclature “(ATOM)ᵢ₋(ATOM)ⱼ” with j>i, when applied herein to a class of substituents, is meant to refer to embodiments of this disclosure for which each and every one of the number of atom members, from i to j including i and j, is independently realized. By way of example, the term C₁-C₃ refers independently to embodiments that have one carbon member (C₁), embodiments that have two carbon members (C₂), and embodiments that have three carbon members (C₃).

Any disubstituent referred to herein is meant to encompass the various attachment possibilities when more than one of such possibilities are allowed. For example, reference to disubstituent -J-K—, where J≠K, refers herein to such disubstituent with J attached to a first substituted member and K attached to a second substituted member, and it also refers to such disubstituent with J attached to the second substituted member and K attached to the first substituted member.

It will be appreciated that certain of the compounds described herein include one or more position that can exists as stereoisomers. For example, certain of the compounds described herein include one or more carbon atoms that can exist in one or more stereoisomeric arrangements. It will be appreciated that a carbon atom that can exist in stereoisomeric arrangements that is depicted without showing any stereoisomeric arrangement includes as a disclosure each of eh possible stereoisomeric arrangements. For example a carbon atom having four groups that can be prioritized according to the Cahn-Ingold Prelog Rules known to one of skill in the art will be understood herein as describing no particular stereochemical definition as in the structure on the left below, and also as describing both possible stereoisomers (S) and (R) as shown below

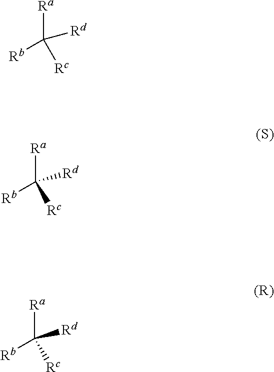

where R^{a}>R^{b}>R^{c}>R^{d} according to the Cahn-Ingold Prelog Rules.

The disclosure also includes pharmaceutically acceptable salts of the compounds represented by Formula (I)-(VI), preferably of those described above and of the specific compounds exemplified herein, and pharmaceutical compositions comprising such salts, and methods of using such salts.

A “pharmaceutically acceptable salt” is intended to mean a salt of a free acid or base of a compound represented herein that is non-toxic, biologically tolerable, or otherwise biologically suitable for administration to the subject. See, generally, S. M. Berge, et al., “Pharmaceutical Salts,” J. Pharm. Sci., 1977, 66, 1-19. Preferred pharmaceutically acceptable salts are those that are pharmacologically effective and suitable for contact with the tissues of subjects without undue toxicity, irritation, or allergic response. A compound described herein may possess a sufficiently acidic group, a sufficiently basic group, both types of functional groups, or more than one of each type, and accordingly react with a number of inorganic or organic bases, and inorganic and organic acids, to form a pharmaceutically acceptable salt.

Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of Formula (I)-(VI) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

The disclosure also relates to pharmaceutically acceptable prodrugs of the compounds of Formula (I)-(VI), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)-(VI)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs,” ed. H. Bundgaard, Elsevier, 1985.

The present disclosure also relates to pharmaceutically active metabolites of compounds of Formula (I)-(VI), and uses of such metabolites in the methods of the disclosure. A “pharmaceutically active metabolite” means a pharmacologically active product of metabolism in the body of a compound of Formula (I)-(VI) or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 255-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

### REPRESENTATIVE EMBODIMENTS

In some embodiments, the disclosure provides a compound of the formula I, or a pharmaceutically acceptable salt thereof,

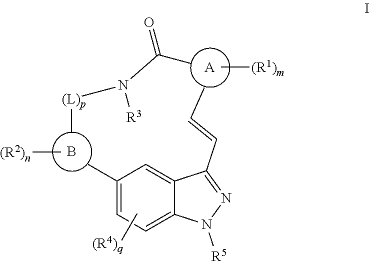

wherein R¹, R², R³, R⁴, R⁵, A, B, L, m, n, p, and q are as described herein.

In some embodiments, the disclosure provides a compound of the formula II, or a pharmaceutically acceptable salt thereof,

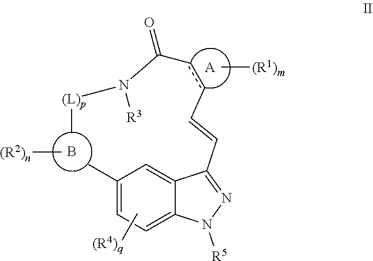

wherein R¹, R², R³, R⁴, R⁵, A, B, L, m, n, p, q, and “ [symbol] ” are as described herein.

In some embodiments, the disclosure provides a compound of the formula III, or a pharmaceutically acceptable salt thereof,

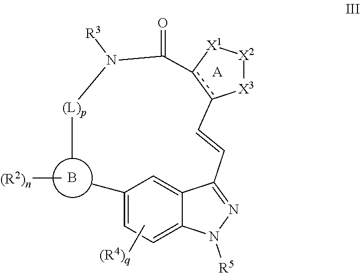

wherein R¹, R², R³, R⁴, R⁵, A, B, L, X¹, X², X³, m, n, p, q, and “ [symbol] ” are as described herein.

In some embodiments, the disclosure provides a compound of the formula IV, or a pharmaceutically acceptable salt thereof,

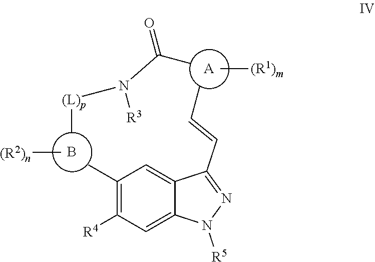

wherein R¹, R², R³, R⁴, R⁵, A, B, L, m, n, and p are as described herein.

In some embodiments, the disclosure provides a compound of the formula V, or a pharmaceutically acceptable salt thereof,

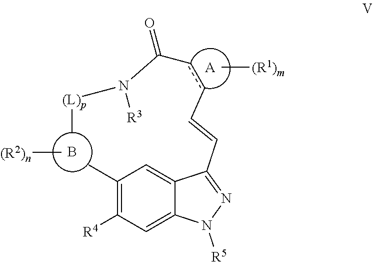

wherein R¹, R², R³, R⁴, R⁵, A, B, L, m, n, p, and “ [symbol] ” are as described herein.

In some embodiments, the disclosure provides a compound of the formula VI, or a pharmaceutically acceptable salt thereof,

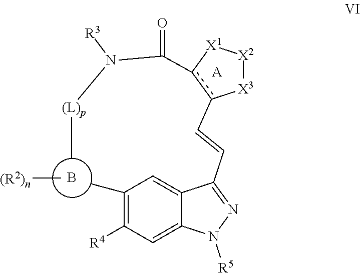

wherein R¹, R², R³, R⁴, R⁵, A, B, L, X¹, X², X³, m, n, p, and “ [symbol] ” are as described herein.

In some embodiments, ring A is a 5- to 10-membered heteroarylene. In some embodiments, ring A is a 5- or 6-membered heteroarylene. In some embodiments, ring A is a 5-membered heteroarylene. In some embodiments, ring A is a 6-membered heteroarylene. In some embodiments, ring A is a fused bicyclic 8- to 10-membered heteroarylene.

In some embodiments, ring A is a 5- to 10-membered heteroarylene, such as a monocyclic 5- or 6-membered heteroarylene or a bicyclic 8- to 10-membered heteroarylene, wherein each hydrogen atom in the 5- to 10-membered heteroarylene, as described herein, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is pyrrolylene, isoxazolylene, isothiazolylene, pyrazolylene, or imidazolylene, wherein each hydrogen atom in pyrrolylene, isoxazolylene, isothiazolylene, pyrazolylene, and imidazolylene, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is pyridinylene, pyrazinylene, pyrimidinylene, pyridazineylene, or triazinylene, wherein each hydrogen atom in pyridinylene, pyrazinylene, pyrimidinylene, pyridazineylene, and triazinylene, is independently optionally substituted by an R¹ that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is a 5- to 10-membered heteroarylene, such as a monocyclic 5- or 6-membered heteroarylene or a bicyclic 8- to 10-membered heteroarylene, wherein the 5- to 10-membered heteroarylene, as described herein, is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, and —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring A is pyrrolylene, isoxazolylene, isothiazolylene, pyrazolylene, or imidazolylene, wherein each is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, and —NO₂.

In some embodiments, ring A is of the formula

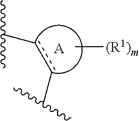

wherein “ [symbol] ” is optionally a carbon-carbon single bond or a carbon-carbon double bond, each “ [symbol] ” represents a point of covalent attachment, and R¹ and m are as described herein. In some embodiments, ring A is of the formula

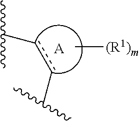

wherein “ [symbol] ” is optionally a carbon-carbon single bond or a carbon-carbon double bond, each “ [symbol] ” represents a point of covalent attachment, ring A is a 5-membered heteroarylene, and R¹ and m are as described herein.

In some embodiments, ring A is of the formula

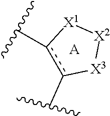

wherein “ [symbol] ” is optionally a carbon-carbon single bond or a carbon-carbon double bond, each “ [symbol] ” represents a point of covalent attachment, X¹, X², and X³ are each independently —O—, —S—, ═C(H)—, ═C(R¹)—, —N(H)—, —N(R′)— or ═N—, provided that at least one of X¹, X², and X³ is not ═C(H)—, or ═C(R′)—, ring A is a 5-membered heteroarylene, and R¹ and m are as described herein.

In some embodiments, ring A is a pyridinylene, pyrazinylene, pyrimidinylene, pyridazineylene, or triazinylene, wherein each is optionally substituted with 1, 2, 3, 4, or 5 of R¹ (m of R¹), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, m is 0, 1, 2, 3, or 4. In some embodiments, m is 0, 1, 2, or 3. In some embodiments, m is 0, 1, or 2. In some embodiments, m is 0 or 1. In some embodiments, m is 0. In some embodiments, m is 1. In some embodiments, m is 2. In some embodiments, m is 3. In some embodiments, m is 4.

In some embodiments, ring A is a 5-membered heteroarylene selected from the group consisting of

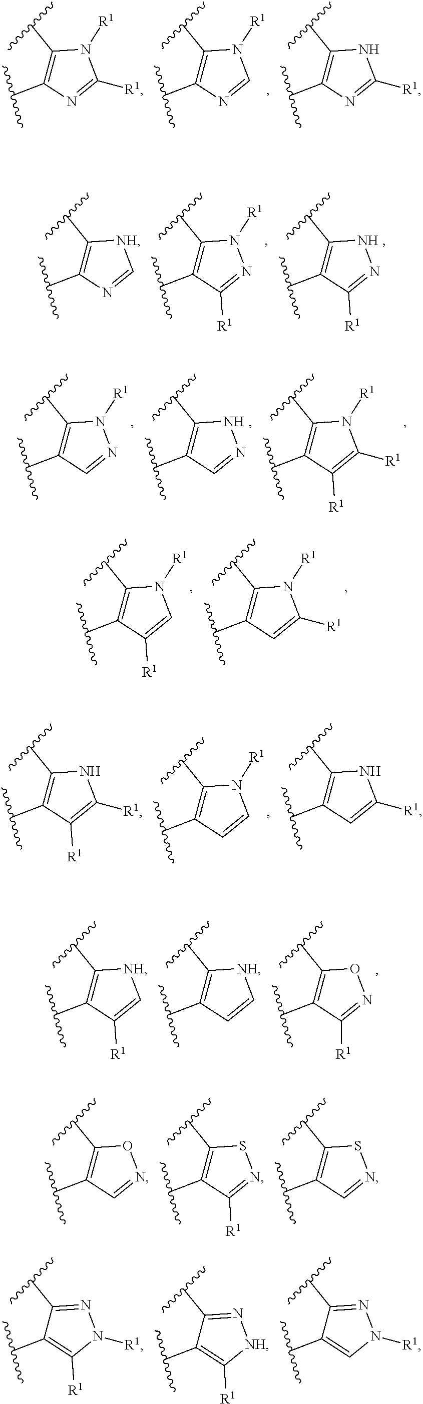

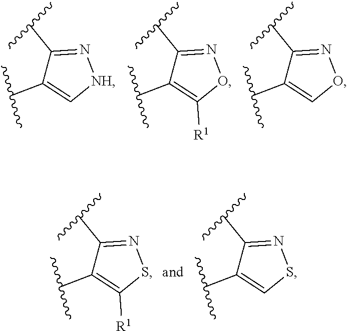

wherein each “ [symbol] ” represents a point of covalent attachment, and each R¹ is independently as described herein.

In some embodiments, ring A is a 5-membered heteroarylene selected from the group consisting of

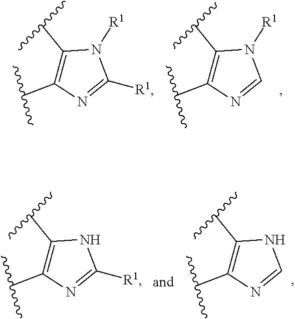

wherein each “ [symbol] ” represents a point of covalent attachment, and each R¹ is independently as described herein.

In some embodiments, ring A is a 5-membered heteroarylene selected from the group consisting of

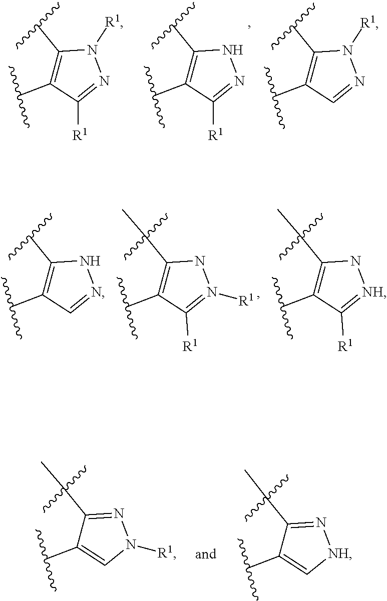

wherein each “ [symbol] ” represents a point of covalent attachment, and each R¹ is independently as described herein.

In some embodiments, ring A is a 5-membered heteroarylene selected from the group consisting of

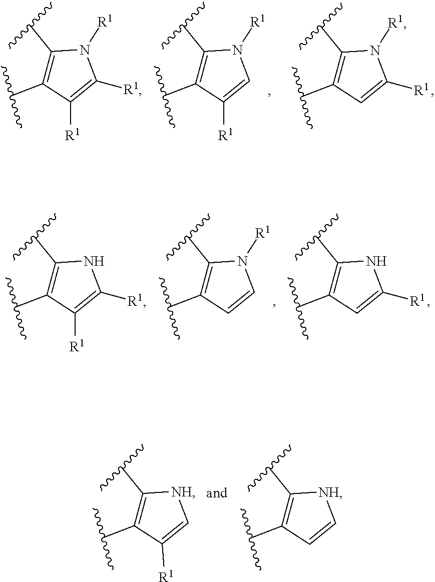

wherein each “ [symbol] ” represents a point of covalent attachment, and each R¹ is independently as described herein.

In some embodiments, ring A is a 5-membered heteroarylene selected from the group consisting of

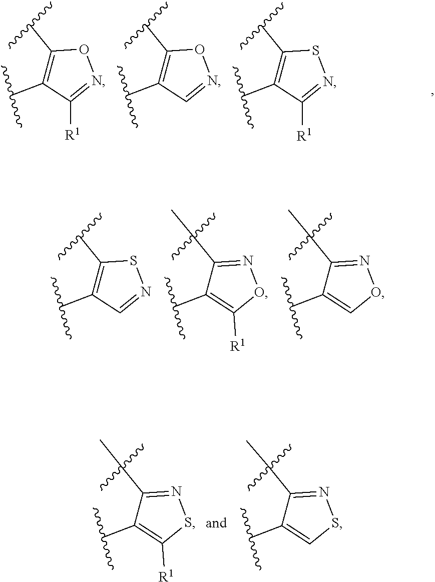

wherein each “ [symbol] ” represents a point of covalent attachment, and R¹ is as described herein.

In some embodiments, ring A is a 5-membered heteroarylene selected from the group consisting of

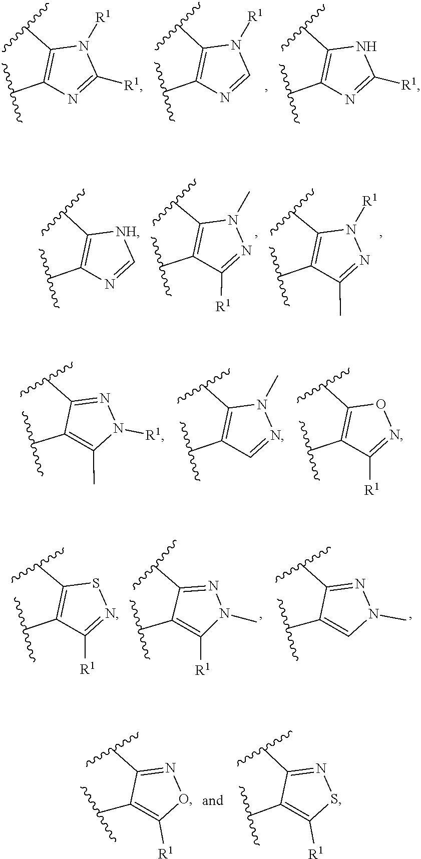

wherein each “ [symbol] ” represents a point of covalent attachment, and each R¹ is independently as described herein.

In some embodiments, ring A is a 5-membered heteroarylene selected from the group consisting of

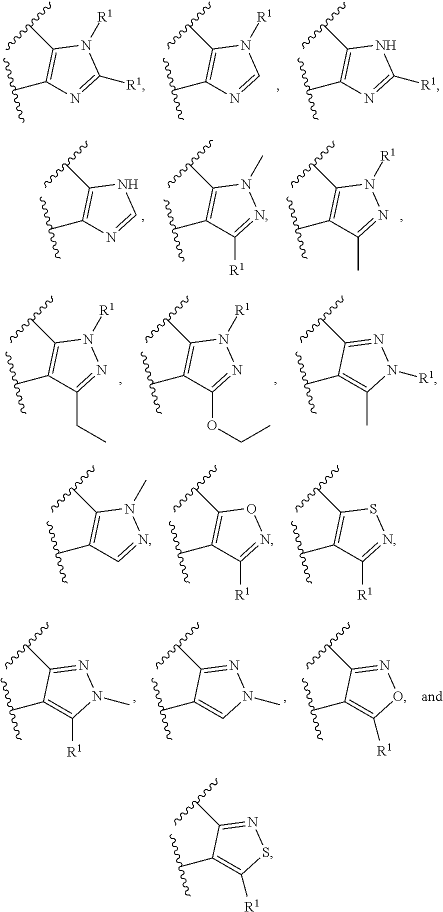

wherein each “ [symbol] ” represents a point of covalent attachment.

In some embodiments, each R¹ is independently deuterium, halogen, or C₁-C₆ alkyl, wherein each hydrogen atom in C₁-C₆ alkyl is independently optionally substituted deuterium, halogen, C₁-C₆ alkyl, C₁-C₆ haloalkyl, —OR^{C}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R¹ is each R¹ is independently methyl, ethyl, F, Cl, Br,

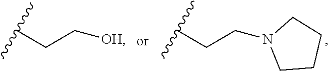

wherein “ [symbol] ” represents a point of covalent attachment

In some embodiments, Ring A is a 5-membered heteroarylene selected from the group consisting of

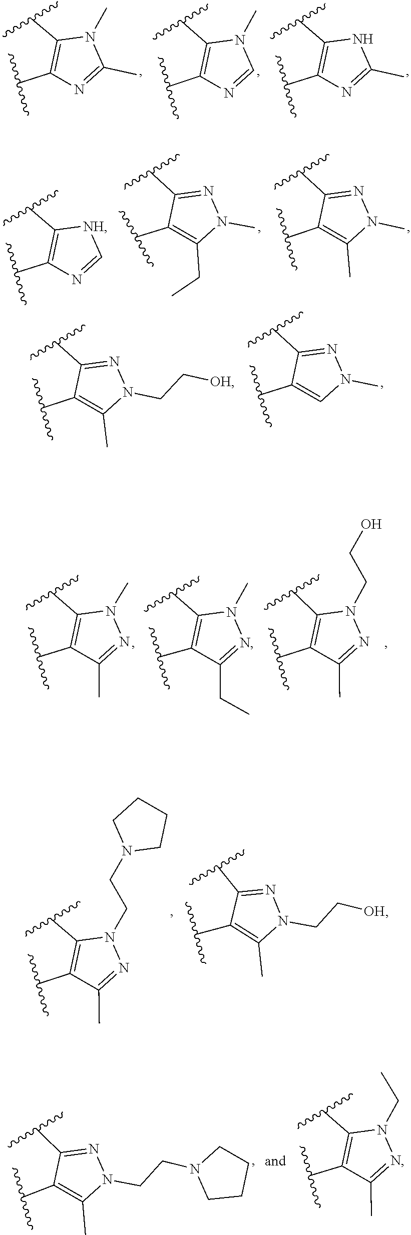

wherein each “ [symbol] ” represents a point of covalent attachment.

In some embodiments, ring A is a 5-membered heteroarylene selected from the group consisting of

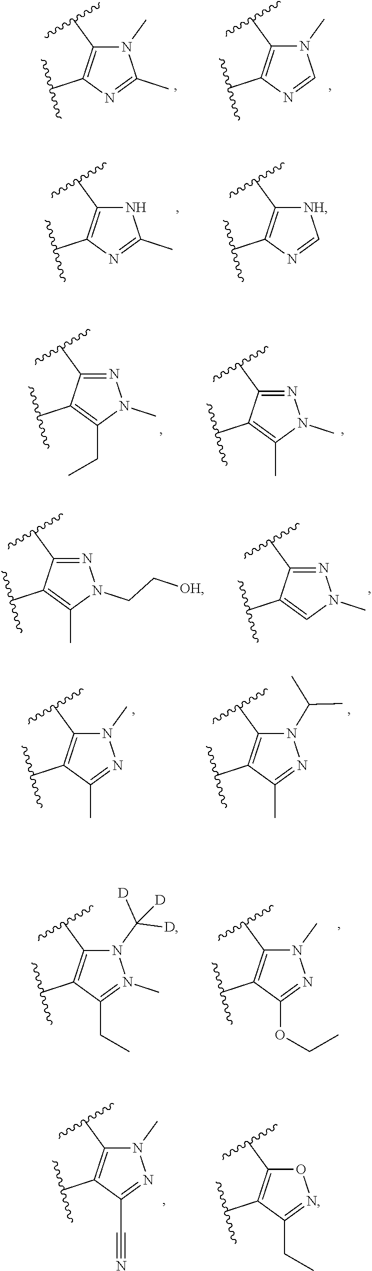

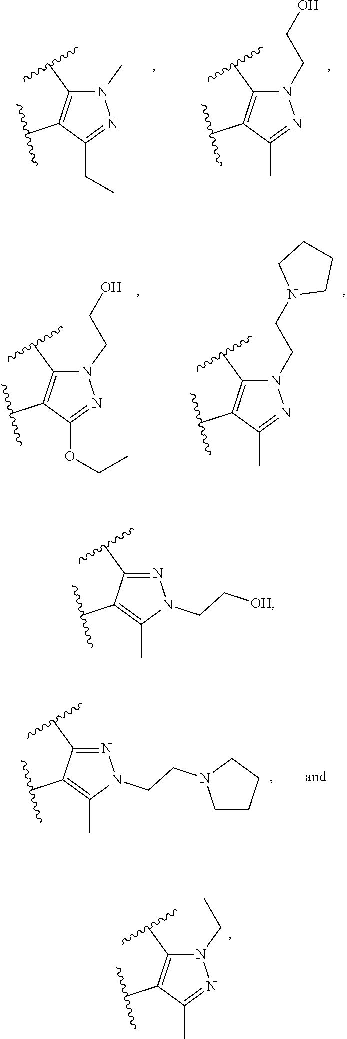

wherein each “ [symbol] ” represents a point of covalent attachment.

In some embodiments, ring B is a 5- to 10-membered heteroarylene or a C₆-C₁₀ arylene. In some embodiments, Ring B is mono- or bi-cyclic C₆-C₁₀ arylene or mono- or bi-cyclic 5- to 10-membered heteroarylene.

In some embodiments, ring B is a 5- to 10-membered heteroarylene. In some embodiments, ring B is a 5- or 6-membered heteroarylene. In some embodiments, ring B is a 5-membered heteroarylene. In some embodiments, ring B is a 6-membered heteroarylene. In some embodiments, ring B is a fused bicyclic 8- to 10-membered heteroarylene.

In some embodiments, ring B is a 5- to 10-membered heteroarylene, such as a monocyclic 5- or 6-membered heteroarylene or a bicyclic 8- to 10-membered heteroarylene, wherein each hydrogen atom in the 5- to 10-membered heteroarylene, as described herein, is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is isoxazolylene, isothiazolylene, or pyrazolylene, wherein each hydrogen atom in isoxazolylene, isothiazolylene, or pyrazolylene, and imidazolylene, is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is isoxazolylene, isothiazolylene, or pyrazolylene, wherein each hydrogen atom in isoxazolylene, isothiazolylene, or pyrazolylene, is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is a 5- to 10-membered heteroarylene, such as a monocyclic 5- or 6-membered heteroarylene or a bicyclic 8- to 10-membered heteroarylene, wherein the 5- to 10-membered heteroarylene, as described herein, is optionally substituted with 1, 2, 3, 4, or 5 of R² (m of R²), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is isoxazolylene, isothiazolylene, or pyrazolylene, wherein each is optionally substituted with 1, 2, 3, 4, or 5 of R² (m of R²), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, n is 0, 1, 2, 3, or 4. In some embodiments, n is 0, 1, 2, or 3. In some embodiments, n is 0, 1, or 2. In some embodiments, n is 0 or 1. In some embodiments, n is 0. In some embodiments, n is 1. In some embodiments, n is 2. In some embodiments, n is 3. In some embodiments, n is 4.

In some embodiments, ring B is a 5-membered heteroarylene selected from the group consisting of

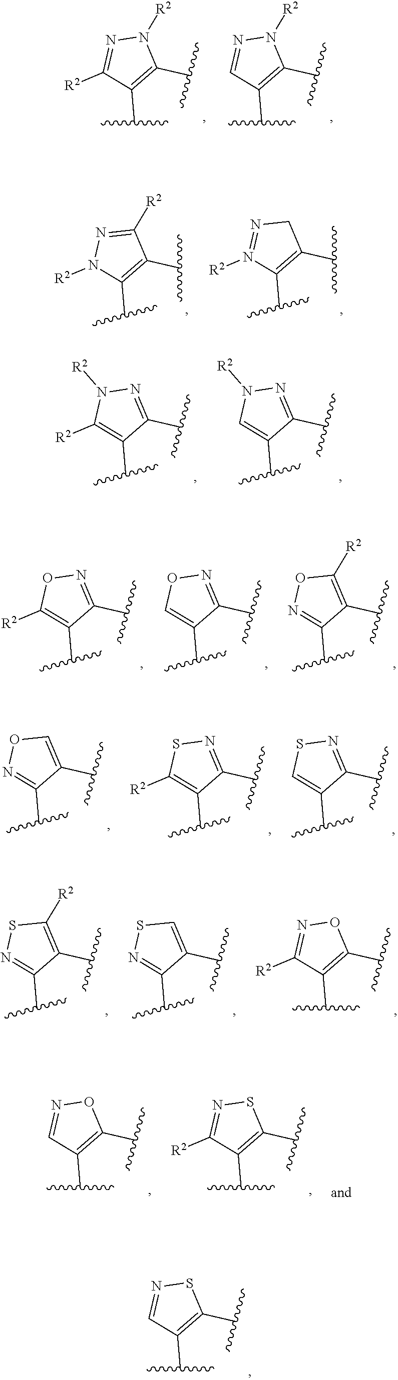

wherein each “ [symbol] ” represents a point of covalent attachment, and each R² is independently as described herein.

In some embodiments, R² is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R² is methyl, or ethyl.

In some embodiments, ring B is a 5-membered heteroarylene selected from the group consisting of

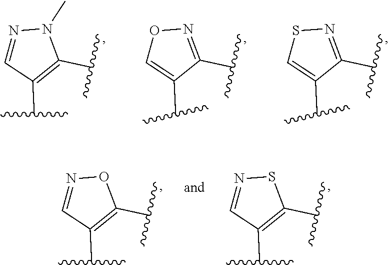

wherein each “ [symbol] ” represents a point of covalent attachment.

In some embodiments, Ring B is mono- or bi-cyclic C₆-C₁₀ arylene. In some embodiments, Ring B is monocyclic C₆-C₁₀ arylene. In some embodiments, Ring B is bicyclic C₆-C₁₀ arylene.

In some embodiments, Ring B is a C₆-C₁₀ mono- or bi-cyclic arylene, wherein each hydrogen atom in C₆-C₁₀ mono- or bi-cyclic arylene is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, Ring B is phenylene or naphthylene, wherein each hydrogen atom in phenylene or naphthylene is independently optionally substituted by an R² that is deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is a C₆-C₁₀ mono- or bi-cyclic arylene, wherein each is optionally substituted with 1, 2, 3, 4, or 5 of R² (n of R²), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, and —NO₂.

In some embodiments, ring B is phenylene or naphthylene, wherein each is optionally substituted with 1, 2, 3, 4, or 5 of R² (n of R²), each of which is independently selected from the group consisting of deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂.

In some embodiments, ring B is a C₆-C₁₀ arylene, and n is as defined herein. In some embodiments, ring B is a phenylene, and n is as defined herein.

In some embodiments, n is 0, 1, 2, 3, or 4. In some embodiments, n is 0, 1, 2, or 3. In some embodiments, n is 0, 1, or 2. In some embodiments, n is 0 or 1. In some embodiments, n is 0. In some embodiments, n is 1. In some embodiments, n is 2. In some embodiments, n is 3. In some embodiments, n is 4.

In some embodiments, R² is methyl, ethyl, F, Cl, or Br. In some embodiments, ring B is a phenylene, n is 1, and R² is methyl, ethyl, F, Cl, or Br.

In some embodiments, ring B is of the formula

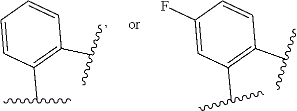 [SMILES: *c1ccccc1* ; *c1ccc(F)cc1*]

wherein each “ [symbol] ” represents a point of covalent attachment.

In some embodiments, R³ is H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{C}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R³ is H or C₁-C₆ alkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{C}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R³ is H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by deuterium, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂. In some embodiments, R³ is H or C₁-C₆ alkyl. In some embodiments, R³ is H or methyl.

In some embodiments, q is 0, 1, or 2. In some embodiments, q is 0 or 1. In some embodiments, q is 0. In some embodiments, q is 1. In some embodiments, q is 2.

In some embodiments, each R⁴ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{c}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂. In some embodiments, each R⁴ is independently deuterium, halogen, or C₁-C₆ alkyl. In some embodiments, each R⁴ is H, fluoro, chloro, or methyl.

In some embodiments, R⁵ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR^{c}. In some embodiments, R⁵ is H deuterium. In some embodiments, R⁵ is —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR^{c}. In some embodiments, R⁵ is C₁-C₆ alkyl. In some embodiments, R⁵ is methyl or ethyl.

In some embodiments, each L is independently —O—, —S—, —S(O)—, —S(O)₂—, —N(R⁶)C(O)—, —C(O)N(R⁶)—, —N(R⁶)—, —N(R⁶)S(O)—, —S(O)N(R⁶)—, —N(R⁶)S(O)₂—, —S(O)₂N(R⁶)—, or —C(R⁷)(R⁸)—, provided that (L)ₚ does not comprise an O—O, S—O, or N—N bond. In some embodiments, each L is independently each L is independently —C(R⁷)(R⁸)—, —C(O)—, —O—, or —N(R⁶)—, provided that (L)ₚ does not comprise a —O—O— or a —O—N(R⁶)— bond, and the point of covalent attachment of (L)ₚ to —NR³— does not form a —N—N— or a —O—N— bond.

In some embodiments, p is 3, 4, 5, 6, 7, 8, or 9. In some embodiments, p is 5, 6, 7, 8, or 9. In some embodiments, p is 4, 5, 6, 7, or 8. In some embodiments, p is 5, 6, 7, or 8. In some embodiments, p is 6, 7, 8, or 9. In some embodiments, p is 5, 6, or 7. In some embodiments, p is 3, 4, 5, 6, or 7. In some embodiments, p is 3, 4, 5, or 6. In some embodiments, p is 3. In some embodiments, p is 4. In some embodiments, p is 5. In some embodiments, p is 6. In some embodiments, p is 7. In some embodiments, p is 8. In some embodiments, p is 9.

In some embodiments, -(L)ₚ- comprises —(CR⁷R⁸)C(O)N(R⁶)—(CR⁷R⁸)₂—, —(CR⁷R⁸)N(R⁶)C(O)—(CR⁷R⁸)₂—, —N(R⁶)—C(O)(CR⁷R⁸)₂O(CR⁷R⁸)₂—, —CR⁷R⁸O(CR⁷R⁸)₂O—(CR⁷R⁸)₂, —O(CR⁷R⁸)₂O(CR⁷R⁸)₂—, —CR⁷R⁸O—CR⁷R⁸—C(O)N(R⁶)—(CR⁷R⁸)₂—, —(CR⁷R⁸)₃O(CR⁷R⁸)₂—, —(CR⁷R⁸)₂O(CR⁷R⁸)₃—, —CR⁷R⁸—N(R⁶)—(CR⁷R⁸)₂—, —CR⁷R⁸—N(R⁶)—(CR⁷R⁸)₃—, —O(CR⁷R⁸)₂O(CR⁷R⁸)₃—, —(CR⁷R⁸)₂—N(R⁶)—(CR⁷R⁸)₃—, —(CR⁷R⁸)₂—N(R⁶)—(CR⁷R⁸)₂—, —O—(CR⁷R⁸)₂—, —O—(CR⁷R⁸)₃—, or —O—(CR⁷R⁸)₄—.

In some embodiments, -(L)ₚ- is —(CR⁷R⁸)C(O)N(R⁶)—(CR⁷R⁸)₂—, —(CR⁷R⁸)N(R⁶)C(O)—(CR⁷R⁸)₂—, —N(R⁶)—C(O)(CR⁷R⁸)₂O(CR⁷R⁸)₂—, —CR⁷R⁸O(CR⁷R⁸)₂O—(CR⁷R⁸)₂, —O(CR⁷R⁸)₂O(CR⁷R⁸)₂—, —CR⁷R⁸O—CR⁷R⁸—C(O)N(R⁶)—(CR⁷R⁸)₂—, —(CR⁷R⁸)₃O(CR⁷R⁸)₂—, —(CR⁷R⁸)₂O(CR⁷R⁸)₃—, —CR⁷R⁸—N(R⁶)—(CR⁷R⁸)₂—, —CR⁷R⁸—N(R⁶)—(CR⁷R⁸)₃—, —O(CR⁷R⁸)₂O(CR⁷R⁸)₃—, —(CR⁷R⁸)₂—N(R⁶)—(CR⁷R⁸)₃—, —(CR⁷R⁸)₂—N(R⁶)—(CR⁷R⁸)₂—, —O—(CR⁷R⁸)₂—, —O—(CR⁷R⁸)₃—, or —O—(CR⁷R⁸)₄—.

In some embodiments, R⁶, when present, is H or C₁-C₆ alkyl. In some embodiments, R⁶, when present, is H or methyl. In some embodiments, R⁷, when present, is H, C₁-C₆ alkyl, —OH, or —OCH₃. In some embodiments, R⁷, when present, is H, methyl, —OH, or —OCH₃. In some embodiments, R⁸, when present, is H, C₁-C₆ alkyl, —OH, or —OCH₃. In some embodiments, R⁸, when present, is H, methyl, —OH, or —OCH₃. In some embodiments, each R⁷ and R⁸, when present, is H.

In some embodiments, -(L)ₚ- is —CH₂C(O)N(H)—(CH₂)₂O—, —CH₂C(O)N(CH₃)—(CH₂)₂O—, —CH₂C(O)N(CH₂CH₃)—(CH₂)₂O—, —CH₂N(H)C(O)—(CH₂)₂O—, —CH₂C(O)N(CH₃)C(O)—(CH₂)₂O—, —CH₂C(O)N(CH₂CH₃)C(O)—(CH₂)₂O—, —C(O)N(H)—(CH₂)₂O(CH₂)₂—, —N(H)—C(O)(CH₂)₂O(CH₂)₂—, —CH₂O(CH₂)₃O—, —CH₂O(CH₂)₂OCH₂—, —(CH₂)₂O(CH₂)₂O—, —CH₂O—CH₂—C(O)N(H)—(CH₂)₂—, —CH₂O(CH₂)₂C(O)N(H)—CH₂—, —CH₂O(CH₂)₂N(H)C(O)—, —CH₂O(CH₂)₃N(H)C(O)—, —(CH₂)₂O(CH₂)₂N(H)C(O)—, —CH(CH₃)—CH₂O(CH₂)₂N(CH₃)C(O)—, —CH(CH₃)—CH₂O(CH₂)₂N(H)C(O)—, —CH(OCH₃)—CH₂O(CH₂)₂N(CH₃)C(O)—, —CH(OCH₃)—CH₂O(CH₂)₂N(H)C(O)—, —O(CH₂)₂O(CH₂)₂N(H)C(O)—, —CH₂O(CH₂)₂N(H)C(O)—CH₂—, or —O—(CH₂)₃C(O)N(H)—.

In some embodiments, -(L)ₚ- is —CH₂C(O)N(H)—(CH₂)₂OCH₂—, —C(O)N(H)—(CH₂)₂O(CH₂)₂—, —N(H)—C(O)(CH₂)₂O(CH₂)₂—, —CH₂O(CH₂)₂O—(CH₂)₂, —O(CH₂)₂O(CH₂)₂O—, —CH₂O—CH₂—C(O)N(H)—(CH₂)₂—, —CH₂O(CH₂)₂C(O)N(H)—CH₂—, —CH₂O(CH₂)₂N(H)C(O)—, —CH₂O(CH₂)₃N(H)C(O)—, —(CH₂)₂O(CH₂)₂N(H)C(O)—, —CH(CH₃)—CH₂O(CH₂)₂N(CH₃)C(O)—, —CH(CH₃)—CH₂O(CH₂)₂N(H)C(O)—, —CH(OCH₃)—CH₂O(CH₂)₂N(CH₃)C(O)—, —CH(OCH₃)—CH₂O(CH₂)₂N(H)C(O)—, —O(CH₂)₂O(CH₂)₂N(H)C(O)—, —CH₂O(CH₂)₂N(H)C(O)—CH₂—, or —O—(CH₂)₃C(O)N(H)—.

In some embodiments, -(L)ₚ- comprises —CH₂N(H)—(CH₂)₂—, —CH₂N(CH₃)—(CH₂)₂—, —O(CH₂)₂—, —O(CH₂)₃—, —O(CH₂)₄—, and —O(CH₂)₂O(CH₂)₂—.

In some embodiments, -(L)ₚ- is —CH₂N(H)—(CH₂)₂—, —CH₂N(CH₃)—(CH₂)₂—, —O(CH₂)₂—, —O(CH₂)₃—, —O(CH₂)₄—, and —O(CH₂)₂O(CH₂)₂—.

In some embodiments, -(L)ₚ- is —CH₂N(H)—(CH₂)₂—, —CH₂N(CH₃)—(CH₂)₂—, —O(CH₂)₂—, —OCH(CH₃)CH₂—, —O(CH₂)₃—, —O(CH₂)₄—, and —O(CH₂)₂O(CH₂)₂—.

In some embodiments, the disclosure provides a compound selected from the group consisting of

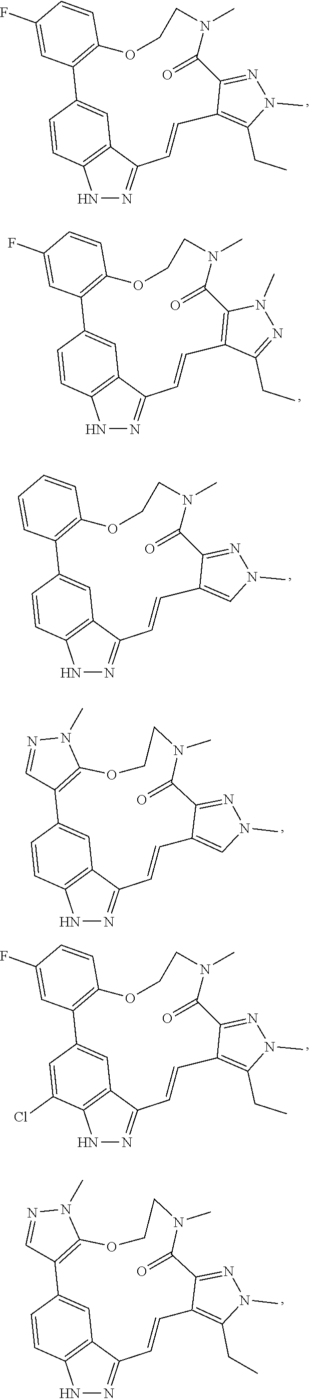

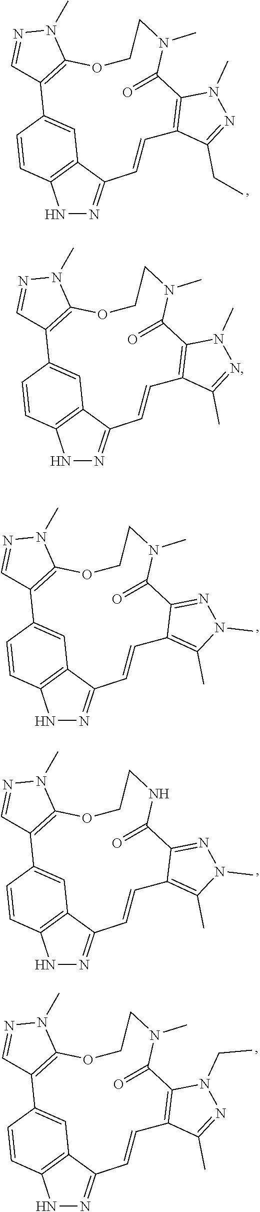

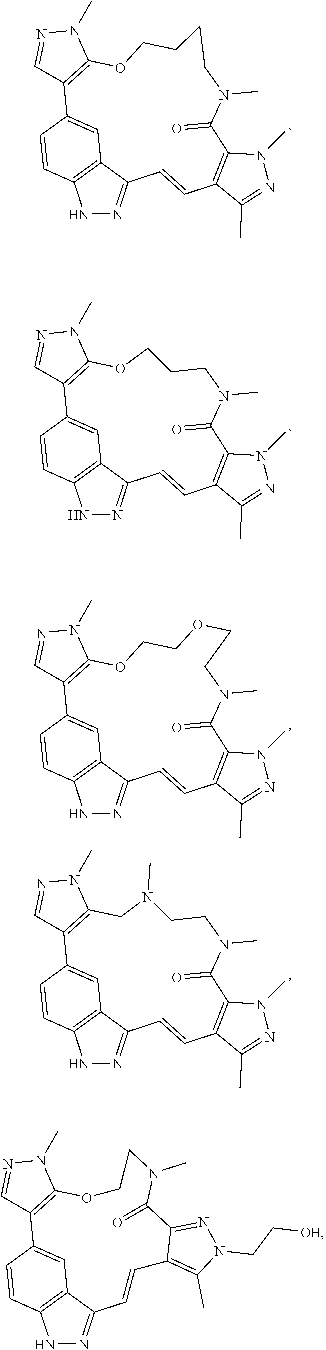

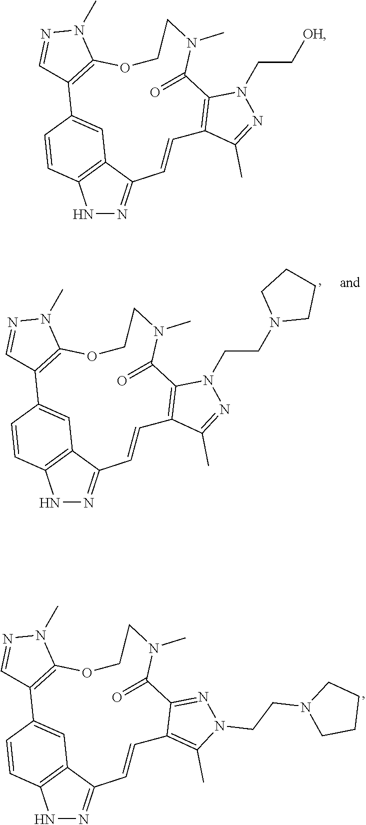

or a pharmaceutically acceptable salt thereof.

In some embodiments, the disclosure provides a compound selected from the group consisting of (17E)-8,12,15-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-16-ethyl-8,12,14-trimethyl-2,11,12,14-tetrahydro-8H-3,5-ethenotripyrazolo|3,4-f:3′,4′-j:4″,3″-n∥1,4|oxazacyclopentadecin-13(10H)-one;
(17E)-16-ethyl-8,12,15-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-8,12,14,16-tetramethyl-2,11,12,14-tetrahydro-8H-3,5-ethenotripyrazolo[3,4-f:3′,4′-j:4″,3″-n][1,4]oxazacyclopentadecin-13(10H)-one;
(17E)-8,12,15,16-tetramethyl-2,11,12,15-tetrahydro-8H-3,5-ethenotripyrazolo[3,4-f:3′,4′-j:4″,3″-n][1,4]oxazacyclopentadecin-13(10H)-one;
(17E)-8,15,16-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-14-ethyl-8,12,16-trimethyl-2,11,12,14-tetrahydro-8H-3,5-ethenotripyrazolo[3,4-f:3′,4′-j:4″,3″-n][1,4]oxazacyclopentadecin-13(10H)-one;
(19E)-8,14,16,18-tetramethyl-2,11,12,13,14,16-hexahydro-8H-3,5-ethenotripyrazolo[3,4-h:3′,4′-l:4″,3″-p][1,6]oxazacycloheptadecin-15(10H)-one;
(18E)-8,13,15,17-tetramethyl-2,10,11,12,13,15-hexahydro-3,5-ethenotripyrazolo[3,4-g:3′,4′-k:4″,3″-o][1,5]oxazacyclohexadecin-14(8H)-one;
(15E)-3,10,12,14-tetramethyl-5,6,9,10,12,18-hexahydro-3H-19,21-ethenotripyrazolo[3,4-i:3′,4′-m:4″,3″-q][1,4,7]dioxazacyclooctadecin-11(8H)-one;
(18E)-8,10,13,15,17-pentamethyl-2,8,9,10,11,12,13,15-octahydro-14H-3,5-ethenotripyrazolo[3,4-f:3′,4′-j:4″,3″-n][1,4]diazacyclohexadecin-14-one;
(17E)-14-(2-hydroxyethyl)-8,12,16-trimethyl-2,11,12,14-tetrahydro-8H-3,5-ethenotripyrazolo[3,4-f:3′,4′-j:4″,3″-n][1,4]oxazacyclopentadecin-13(10H)-one;
(17E)-15-(2-hydroxyethyl)-8,12,16-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-8,12,16-trimethyl-14-[2-(pyrrolidin-1-yl)ethyl]-2,11,12,14-tetrahydro-8H-3,5-ethenotripyrazolo[3,4-f:3′,4′-j:4″,3″-n][1,4]oxazacyclopentadecin-13(10H)-one; and
(17E)-8,12,16-trimethyl-15-[2-(pyrrolidin-1-yl)ethyl]-2,11,12,15-tetrahydro-8H-3,5-ethenotripyrazolo[3,4-f:3′,4′-j:4″,3″-n][1,4]oxazacyclopentadecin-13(10H)-one
   or a pharmaceutically acceptable salt thereof.

In some embodiments, the disclosure provides a compound selected from the group consisting of (18E)-17-ethyl-7-fluoro-13,16-dimethyl-2,12,13,16-tetrahydro-3,5-ethenodipyrazolo[3,4-f3′,4′-j][1,4]benzoxazacyclopentadecin-14(11H)-one;
(18E)-17-ethyl-7-fluoro-13,15-dimethyl-2,12,13,15-tetrahydro-3,5-ethenodipyrazolo[3,4-f:3′,4′-j][1,4]benzoxazacyclopentadecin-14(11H)-one
(18E)-13,16-dimethyl-2,12,13,16-tetrahydro-3,5-ethenodipyrazolo[3,4-f:3′,4′-j][1,4]benzoxazacyclopentadecin-14(11H)-one; and
(18E)-21-chloro-17-ethyl-7-fluoro-13,16-dimethyl-2,12,13,16-tetrahydro-3,5-ethenodipyrazolo[3,4-f:3′,4′-j][1,4]benzoxazacyclopentadecin-14(11H)-one;
   or a pharmaceutically acceptable salt thereof.

In some embodiments, the disclosure provides a compound selected from the group consisting of (17E)-8,12,15-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-16-ethyl-8,12,14-trimethyl-2,11,12,14-tetrahydro-8H-3,5-ethenotripyrazolo[3,4-f:3′,4′-j:4″,3″-n][1,4]oxazacyclopentadecin-13(10H)-one;
(17E)-16-ethyl-8,12,15-trimethyl-2,11,12,15-tetrahydro-8H-3,5-ethenotripyrazolo[3,4-f:3′,4-j:4″,3″-n][1,4]oxazacyclopentadecin-13(10H)-one;
(17E)-8,12,14,16-tetramethyl-2,11,12,14-tetrahydro-8H-3,5-ethenotripyrazolo[3,4-f3′,4-j:4″,3″-n∥1,4|oxazacyclopentadecin-13(10H)-one;
(17E)-8,12,15,16-tetramethyl-2,11,12,15-tetrahydro-8H-3,5-ethenotripyrazolo[3,4-f:3′,4′-j:4″,3″-n][1,4]oxazacyclopentadecin-13(10H)-one;
(17E)-8,15,16-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-14-ethyl-8,12,16-trimethyl-2,11,12,14-tetrahydro-8H-3,5-ethenotripyrazolo[3,4-f:3′,4′-j:4″,3″-n][1,4]oxazacyclopentadecin-13(10H)-one;
(19E)-8,14,16,18-tetramethyl-2,11,12,13,14,16-hexahydro-8H-3,5-ethenotripyrazolo[3,4-h:3′,4′-l:4″,3″-p][1,6]oxazacycloheptadecin-15(10H)-one;
(18E)-8,13,15,17-tetramethyl-2,10,11,12,13,15-hexahydro-3,5-ethenotripyrazolo[3,4-g:3′,4′-k:4″,3″-o][1,5]oxazacyclohexadecin-14(8H)-one;
(15E)-3,10,12,14-tetramethyl-5,6,9,10,12,18-hexahydro-3H-19,21-ethenotripyrazolo[3,4-i:3′,4′-m:4″,3″-q][1,4,7]dioxazacyclooctadecin-11(8H)-one;
(18E)-8,10,13,15,17-pentamethyl-2,8,9,10,11,12,13,15-octahydro-14H-3,5-ethenotripyrazolo[3,4-f:3′,4′-j:4″,3″-n][1,4]diazacyclohexadecin-14-one;
(17E)-14-(2-hydroxyethyl)-8,12,16-trimethyl-2,11,12,14-tetrahydro-8H-3,5-ethenotripyrazolo[3,4-f:3′,4′-j:4″,3″-n][1,4]oxazacyclopentadecin-13(10H)-one;
(17E)-15-(2-hydroxyethyl)-8,12,16-trimethyl-2,11,12,15-tetrahydro-8H-3,5-ethenotripyrazolo[3,4-f:3′,4′-j:4″,3″-n][1,4]oxazacyclopentadecin-13(10H)-one;
(17E)-8,12,16-trimethyl-14-[2-(pyrrolidin-1-yl)ethyl]-2,11,12,14-tetrahydro-8H-3,5-ethenotripyrazolo[3,4-f:3′,4′-j:4″,3″-n][1,4]oxazacyclopentadecin-13(10H)-one;
(17E)-8,12,16-trimethyl-15-[2-(pyrrolidin-1-yl)ethyl]-2,11,12,15-tetrahydro-8H-3,5-ethenotripyrazolo[3,4-f:3′,4′-j:4″,3″-n][1,4]oxazacyclopentadecin-13(10H)-one;
(17E)-8,12,16-trimethyl-14-(propan-2-yl)-2,11,12,14-tetrahydro-8H-3,5-ethenotripyrazolo[3,4-f:3′,4′-j:4″,3″-n][1,4]oxazacyclopentadecin-13(10H)-one;
(17E)-16-ethyl-8-methyl-12,14-bis[(²H₃)methyl]-2,11,12,14-tetrahydro-8H-3,5-ethenotripyrazolo[3,4-f:3′,4′-j:4″,3″-n][1,4]oxazacyclopentadecin-13(10H)-one;
(17E)-16-ethoxy-8,12,14-trimethyl-2,11,12,14-tetrahydro-8H-3,5-ethenotripyrazolo[3,4-f:3′,4′-j:4″,3″-n][1,4]oxazacyclopentadecin-13(10H)-one;
(10S,17E)-16-ethyl-8,10,12,14-tetramethyl-2,11,12,14-tetrahydro-8H-3,5-ethenotripyrazolo[3,4-f:3′,4′-j:4″,3″-n][1,4]oxazacyclopentadecin-13(10H)-one;
(17E)-8,12,14-trimethyl-13-oxo-2,10,11,12,13,14-hexahydro-8H-3,5-ethenotripyrazolo[3,4-f:3′,4′-j:4″,3″-n][1,4]oxazacyclopentadecine-16-carbonitrile;
(17E)-16-ethyl-8,12-dimethyl-2,8,11,12-tetrahydro-3,5-etheno[1,2]oxazolo[5,4-f|dipyrazolo|3,4-j:4′,3′-n∥1,4|oxazacyclopentadecin-13(10H)-one;
(18E)-15-(2-hydroxyethyl)-8,10,13,17-tetramethyl-2,8,9,10,11,12,13,15-octahydro-14H-3,5-ethenotripyrazolo[3,4-f:3′,4′-j:4″,3″-n][1,4]diazacyclohexadecin-14-one; and
(17E)-16-ethoxy-14-(2-hydroxyethyl)-8,12-dimethyl-2,11,12,14-tetrahydro-8H-3,5-ethenotripyrazolo[3,4-f:3′,4′-j:4″,3″-n][1,4]oxazacyclopentadecin-13(10H)-one;
   or a pharmaceutically acceptable salt thereof.

The following represent illustrative embodiments of compounds of Formula (I):

| Ex. # | Structure | Name |
|---|---|---|
| 1 | 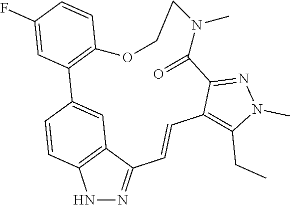 [SMILES: CCc1c2c(nn1C)C(=O)N(C)CCOc1ccc(F)cc1-c1ccc3[nH]nc(c3c1)/C=C/2] | (18E)-17-ethyl-7-fluoro-13,16- dimethyl-2,12,13,16-tetrahydro-3,5- ethenodipyrazolo[3,4-f:3′,4′- j[1,4]benzoxazacyclopentadecin- 14(11H)-one |
| 2 | 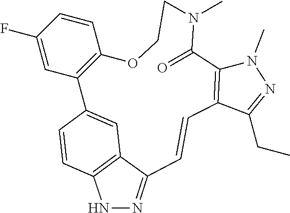 [SMILES: CCc1nn(C)c2c1/C=C/c1n[nH]c3ccc(cc13)-c1cc(F)ccc1OCCN(C)C2=O] | (18E)-17-ethyl-7-fluoro-13,15- dimethyl-2,12,13,15-tetrahydro-3,5- ethenodipyrazolo[3,4-f:3′,4′- j][1,4]benzoxazacyclopentadecin- 14(11H)-one |
| 3 | 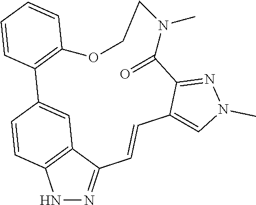 [SMILES: CN1CCOc2ccccc2-c2ccc3[nH]nc(c3c2)/C=C/c2cn(C)nc2C1=O] | (18E)-13,16-dimethyl-2,12,13,16- tetrahydro-3,5-ethenodipyrazolo[3,4- f:3′,4′-j][1,4]benzoxazacyclopentadecin- 14(11H)-one |
| 4 | 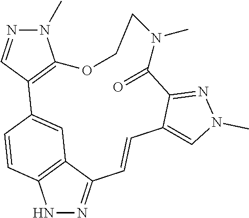 [SMILES: CN1CCOc2c(cnn2C)-c2ccc3[nH]nc(c3c2)/C=C/c2cn(C)nc2C1=O] | (17E)-8,12,15-trimethyl-2,11,12,15- tetrahydro-8H-3,5- ethenotripyrazolo[3,4-f:3′,4′-j:4″,3″- n][1,4]oxazacyclopentadecin-13(10H)- one |
| 5 | 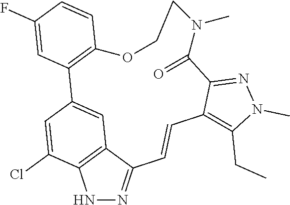 [SMILES: CCc1c2c(nn1C)C(=O)N(C)CCOc1ccc(F)cc1-c1cc(Cl)c3[nH]nc(c3c1)/C=C/2] | (18E)-21-chloro-17-ethyl-7-fluoro- 13,16-dimethyl-2,12,13,16-tetrahydro- 3,5-ethenodipyrazolo[3,4-f:3′,4′- j][1,4]benzoxazacyclopentadecin- 14(11H)-one |
| 6 | 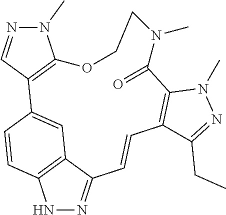 [SMILES: CCc1nn(C)c2c1/C=C/c1n[nH]c3ccc(cc13)-c1cnn(C)c1OCCN(C)C2=O] | (17E)-16-ethyl-8,12,14-trimethyl- 2,11,12,14-tetrahydro-8H-3,5- ethenotripyrazolo[3,4-f:3′,4′-j:4″,3″- n][1,4]oxazacyclopentadecin-13(10H)- one |
| 7 | 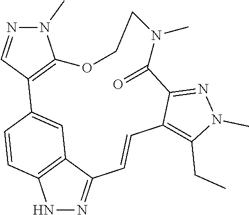 [SMILES: CCc1c2c(nn1C)C(=O)N(C)CCOc1c(cnn1C)-c1ccc3[nH]nc(c3c1)/C=C/2] | (17E)-16-ethyl-8,12,15-trimethyl- 2,11,12,15-tetrahydro-8H-3,5- ethenotripyrazolo[3,4-f:3′,4′-j:4″,3″- n][1,4]oxazacyclopentadecin-13(10H)- one |
| 8 | 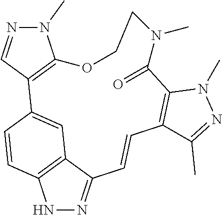 [SMILES: Cc1nn(C)c2c1/C=C/c1n[nH]c3ccc(cc13)-c1cnn(C)c1OCCN(C)C2=O] | (17E)-8,12,14,16-tetramethyl- 2,11,12,14-tetrahydro-8H-3,5- ethenotripyrazolo[3,4-f:3′,4′-j:4″,3″- n][1,4]oxazacyclopentadecin-13(10H)- one |
| 9 | 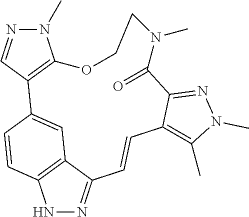 [SMILES: Cc1c2c(nn1C)C(=O)N(C)CCOc1c(cnn1C)-c1ccc3[nH]nc(c3c1)/C=C/2] | (17E)-8,12,15,16-tetramethyl- 2,11,12,15-tetrahydro-8H-3,5- ethenotripyrazolo[3,4-f:3′,4′-j:4″,3″- n][1,4]oxazacyclopentadecin-13(10H)- one |
| 10 | 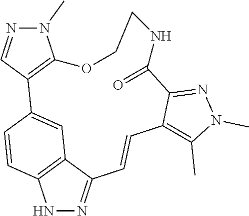 [SMILES: Cc1c2c(nn1C)C(=O)NCCOc1c(cnn1C)-c1ccc3[nH]nc(c3c1)/C=C/2] | (17E)-8,15,16-trimethyl-2,11,12,15- tetrahydro-8H-3,5- ethenotripyrazolo[3,4-f:3′,4′-j:4″,3″- n][1,4]oxazacyclopentadecin-13(10H)- one |
| 11 | 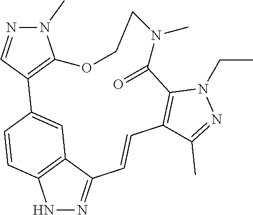 [SMILES: CCn1nc(C)c2c1C(=O)N(C)CCOc1c(cnn1C)-c1ccc3[nH]nc(c3c1)/C=C/2] | (17E)-14-ethyl-8,12,16-trimethyl- 2,11,12,14-tetrahydro-8H-3,5- ethenotripyrazolo[3,4-f:3′,4′-j:4″,3″- n][1,4]oxazacyclopentadecin-13(10H)- one |
| 12 | 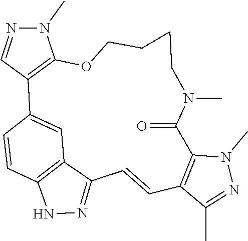 [SMILES: Cc1nn(C)c2c1/C=C/c1n[nH]c3ccc(cc13)-c1cnn(C)c1OCCCCN(C)C2=O] | (19E)-8,14,16,18-tetramethyl- 2,11,12,13,14,16-hexahydro-8H-3,5- ethenotripyrazolo[3,4-h:3′,4′-1:4″,3″- p][1,6]oxazacycloheptadecin-15(10H)- one |
| 13 | 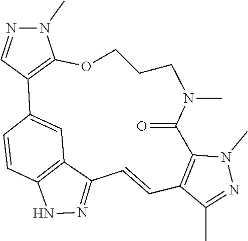 [SMILES: Cc1nn(C)c2c1/C=C/c1n[nH]c3ccc(cc13)-c1cnn(C)c1OCCCN(C)C2=O] | (18E)-8,13,15,17-tetramethyl- 2,10,11,12,13,15-hexahydro-3,5- ethenotripyrazolo[3,4-g:3′,4′-k:4″,3″- o][1,5]oxazacyclohexadecin-14(8H)-one |
| 14 | 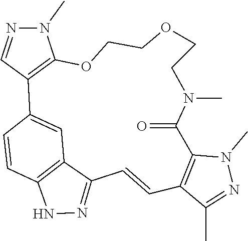 [SMILES: Cc1nn(C)c2c1/C=C/c1n[nH]c3ccc(cc13)-c1cnn(C)c1OCCOCCN(C)C2=O] | (15E)-3,10,12,14-tetramethyl- 5,6,9,10,12,18-hexahydro-3H-19,21- ethenotripyrazolo[3,4-i:3′,4′-m:4″,3″- q][1,4,7]dioxazacyclooctadecin-11(8H)- one |
| 15 | 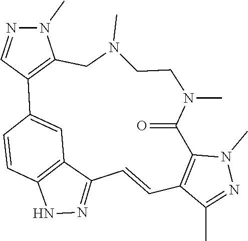 [SMILES: Cc1nn(C)c2c1/C=C/c1n[nH]c3ccc(cc13)-c1cnn(C)c1CN(C)CCN(C)C2=O] | (18E)-8,10,13,15,17-pentamethyl- 2,8,9,10,11,12,13,15-octahydro-14H- 3,5-ethenotripyrazolo[3,4-f:3′,4′-j:4″,3″- n][1,4]diazacyclohexadecin-14-one |
| 16 | 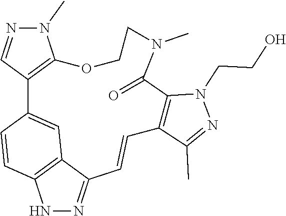 [SMILES: Cc1nn(CCO)c2c1/C=C/c1n[nH]c3ccc(cc13)-c1cnn(C)c1OCCN(C)C2=O] | (17E)-14-(2-hydroxyethyl)-8,12,16- trimethyl-2,11,12,14-tetrahydro-8H-3,5- ethenotripyrazolo[3,4-f:3′,4′-j:4″,3″- n][1,4]oxazacyclopentadecin-13(10H)- one |
| 17 | 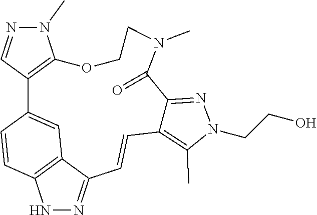 [SMILES: Cc1c2c(nn1CCO)C(=O)N(C)CCOc1c(cnn1C)-c1ccc3[nH]nc(c3c1)/C=C/2] | (17E)-15-(2-hydroxyethyl)-8,12,16- trimethyl-2,11,12,15-tetrahydro-8H-3,5- ethenotripyrazolo[3,4-f:3′,4′-j:4″,3″- n][1,4]oxazacyclopentadecin-13(10H)- one |
| 18 | 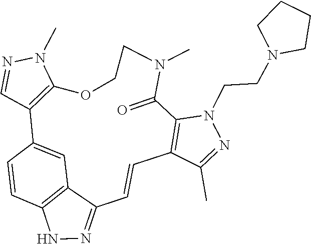 | (17E)-8,12,16-trimethyl-14-[2- (pyrrolidin-1-yl)ethyl]-2,11,12,14- tetrahydro-8H-3,5- ethenotripyrazolo[3,4-f:3′,4′-j:4″,3″- n][1,4]oxazacyclopentadecin-13(10H)- one |
| 19 | 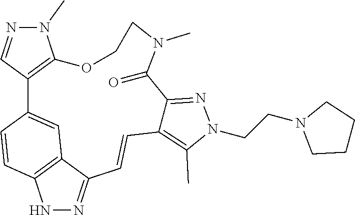 [SMILES: Cc1c2c(nn1CCN1CCCC1)C(=O)N(C)CCOc1c(cnn1C)-c1ccc3[nH]nc(c3c1)/C=C/2] | (17E)-8,12,16-trimethyl-15-[2- (pyrrolidin-1-yl)ethyl]-2,11,12,15- tetrahydro-8H-3,5- ethenotripyrazolo[3,4-f:3′,4′-j:4″,3″- n][1,4]oxazacyclopentadecin-13(10H)- one |
| 20 | 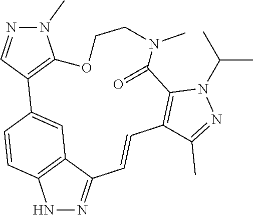 [SMILES: Cc1nn(C(C)C)c2c1/C=C/c1n[nH]c3ccc(cc13)-c1cnn(C)c1OCCN(C)C2=O] | (17E)-8,12,16-trimethyl-14-(propan-2- yl)-2,11,12,14-tetrahydro-8H-3,5- ethenotripyrazolo[3,4-f:3′,4′-j:4″,3″- n][1,4]oxazacyclopentadecin-13(10H)- one |
| 21 | 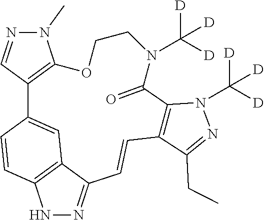 [SMILES: [2H]C([2H])([2H])N1CCOc2c(cnn2C)-c2ccc3[nH]nc(c3c2)/C=C/c2c(CC)nn(C([2H])([2H])[2H])c2C1=O] | (17E)-16-ethyl-8-methyl-12,14- bis[(²H₃)methyl]-2,11,12,14-tetrahydro- 8H-3,5-ethenotripyrazolo[3,4-f:3′,4′- j:4″,3″-n][1,4]oxazacyclopentadecin- 13(10H)-one |
| 22 | 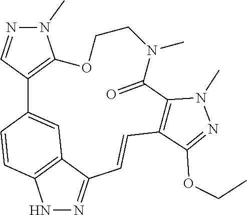 [SMILES: CCOc1nn(C)c2c1/C=C/c1n[nH]c3ccc(cc13)-c1cnn(C)c1OCCN(C)C2=O] | (17E)-16-ethoxy-8,12,14-trimethyl- 2,11,12,14-tetrahydro-8H-3,5- ethenotripyrazolo[3,4-f:3′,4′-j:4″,3″- n][1,4]oxazacyclopentadecin-13(10H)- one |
| 23 | 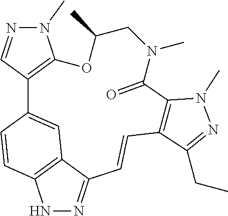 [SMILES: CCc1nn(C)c2c1/C=C/c1n[nH]c3ccc(cc13)-c1cnn(C)c1O[C@@H](C)CN(C)C2=O] | (10S,17E)-16-ethyl-8,10,12,14- tetramethyl-2,11,12,14-tetrahydro-8H- 3,5-ethenotripyrazolo[3,4-f:3′,4′-j:4″,3″- n][1,4]oxazacyclopentadecin-13(10H)- one |
| 24 | 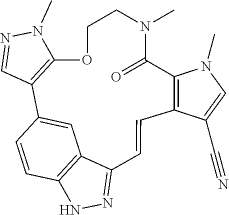 [SMILES: CN1CCOc2c(cnn2C)-c2ccc3[nH]nc(c3c2)/C=C/c2c(C#N)cn(C)c2C1=O] | (17E)-8,12,14-trimethyl-13-oxo- 2,10,11,12,13,14-hexahydro-8H-3,5- ethenotripyrazolo[3,4-f:3′,4′-j:4″,3″- n][1,4]oxazacyclopentadecine-16- carbonitrile |
| 25 | 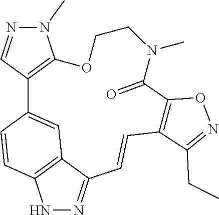 [SMILES: CCc1noc2c1/C=C/c1n[nH]c3ccc(cc13)-c1cnn(C)c1OCCN(C)C2=O] | (17E)-16-ethyl-8,12-dimethyl-2,8,11,12- tetrahydro-3,5-etheno[1,2]oxazolo[5,4- f]dipyrazolo[3,4-j:4′,3′- n][1,4]oxazacyclopentadecin-13(10H)- one |
| 26 | 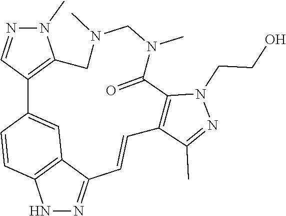 [SMILES: Cc1nn(CCO)c2c1/C=C/c1n[nH]c3ccc(cc13)-c1cnn(C)c1CN(C)CN(C)C2=O] | (18E)-15-(2-hydroxyethyl)-8,10,13,17- tetramethyl-2,8,9,10,11,12,13,15- octahydro-14H-3,5- ethenotripyrazolo[3,4-f:3′,4′-j:4″,3″- n][1,4]diazacyclohexadecin-14-one |
| 27 | 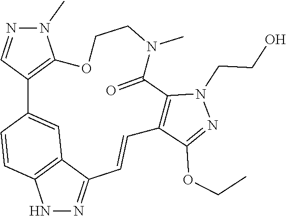 [SMILES: CCOc1nn(CCO)c2c1/C=C/c1n[nH]c3ccc(cc13)-c1cnn(C)c1OCCN(C)C2=O] | (17E)-16-ethoxy-14-(2-hydroxyethyl)- 8,12-dimethyl-2,11,12,14-tetrahydro- 8H-3,5-ethenotripyrazolo[3,4-f:3′,4′- j:4″,3′-n][1,4]oxazacyclopentadecin- 13(10H)-one |

and pharmaceutically acceptable salts thereof.

Those skilled in the art will recognize that the species listed or illustrated herein are not exhaustive, and that additional species within the scope of these defined terms may also be selected.

### Pharmaceutical Compositions

For treatment purposes, pharmaceutical compositions comprising the compounds described herein may further comprise one or more pharmaceutically-acceptable excipients. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the disclosure are sterile compositions. Pharmaceutical compositions may be prepared using compounding techniques known or that become available to those skilled in the art.

Sterile compositions are also contemplated by the disclosure, including compositions that are in accord with national and local regulations governing such compositions.

The pharmaceutical compositions and compounds described herein may be formulated as solutions, emulsions, suspensions, or dispersions in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the disclosure may be administered by a suitable route of delivery, such as oral, parenteral, rectal, nasal, topical, or ocular routes, or by inhalation. Preferably, the compositions are formulated for intravenous or oral administration.

For oral administration, the compounds the disclosure may be provided in a solid form, such as a tablet or capsule, or as a solution, emulsion, or suspension. To prepare the oral compositions, the compounds of the disclosure may be formulated to yield a dosage of, e.g., from about 0.1 mg to 1 g daily, or about 1 mg to 50 mg daily, or about 50 to 250 mg daily, or about 250 mg to 1 g daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil, such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

For parenteral use, including intravenous, intramuscular, intraperitoneal, intranasal, or subcutaneous routes, the agents of the disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 g/kg/minute of agent admixed with a pharmaceutical carrier over a period ranging from several minutes to several days.

For nasal, inhaled, or oral administration, the inventive pharmaceutical compositions may be administered using, for example, a spray formulation also containing a suitable carrier. The inventive compositions may be formulated for rectal administration as a suppository.

For topical applications, the compounds of the present disclosure are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the disclosure may utilize a patch formulation to effect transdermal delivery.

As used herein, the terms “treat” or “treatment” encompass both “preventative” and “curative” treatment. “Preventative” treatment is meant to indicate a postponement of development of a disease, a symptom of a disease, or medical condition, suppressing symptoms that may appear, or reducing the risk of developing or recurrence of a disease or symptom. “Curative” treatment includes reducing the severity of or suppressing the worsening of an existing disease, symptom, or condition. Thus, treatment includes ameliorating or preventing the worsening of existing disease symptoms, preventing additional symptoms from occurring, ameliorating or preventing the underlying systemic causes of symptoms, inhibiting the disorder or disease, e.g., arresting the development of the disorder or disease, relieving the disorder or disease, causing regression of the disorder or disease, relieving a condition caused by the disease or disorder, or stopping the symptoms of the disease or disorder.

The term “subject” refers to a mammalian patient in need of such treatment, such as a human.

Exemplary diseases include cancer, pain, neurological diseases, autoimmune diseases, and inflammation. As used herein, the term “cancer” includes, but is not limited to, ALCL, NSCLC, neuroblastoma, inflammatory myofibroblastic tumor, adult renal cell carcinoma, pediatric renal cell carcinoma, breast cancer, ER⁺ breast cancer, colonic adenocarcinoma, glioblastoma, glioblastoma multiforme, anaplastic thyroid cancer, cholangiocarcinoma, ovarian cancer, gastric adenocarcinoma, colorectal cancer, inflammatory myofibroblastic tumor, angiosarcoma, epithelioid hemangioendothelioma, intrahepatic cholangiocarcinoma, thyroid papillary cancer, spitzoid neoplasms, sarcoma, astrocytoma, brain lower grade glioma, secretory breast carcinoma, mammary analogue carcinoma, myelodysplastic syndromes (MDS), chronic myelomonocytic leukemia (CML), acute myeloid leukemia (AML), congenital mesoblastic nephroma, congenital fibrosarcomas, Ph-like acute lymphoblastic leukemia, thyroid carcinoma, skin cutaneous melanoma, head and neck squamous cell carcinoma, pediatric glioma prostate cancer, lung squamous carcinoma, ovarian serous cystadenocarcinoma, skin cutaneous melanoma, castrate-resistant prostate cancer, Hodgkin lymphoma, and serous and clear cell endometrial cancer. In some embodiments, cancer includes, lung cancer, colon cancer, breast cancer, prostate cancer, hepatocellular carcinoma, renal cell carcinoma, gastric and esophago-gastric cancers, glioblastoma, head and neck cancers, inflammatory myofibroblastic tumors, and anaplastic large cell lymphoma.

In one aspect, the compounds and pharmaceutical compositions of the disclosure specifically target FLT3. Thus, these compounds and pharmaceutical compositions can be used to prevent, reverse, slow, or inhibit diseases, such as cancers driven by the activity of FLT3. In some embodiments, the compounds described herein can target FLT3 in a oncogenic driver mutation, such as FLT3-ITD. In some embodiments, the compounds described herein can target FLT3 (e.g. in such as FLT3-ITD) having one or more resistance mutations, such as such as resistance mutations in the activating loop residues (e.g., D835, 1836, D839, and Y842), or in the gatekeeper residue F691 of FLT3. In some embodiments, methods of treating a target cancer, such as AML, are described.

In one aspect, the compounds and pharmaceutical compositions of the disclosure specifically target PIM kinases. In some embodiments, the compounds described herein can target PIM kinase activity to overcome resistance mechanisms of chemotherapy, radiotherapy, anti-angiogenic therapies and targeted therapies. In some embodiments, methods of treating a target cancer, such as AML, are described.

In one aspect, the compounds and pharmaceutical compositions of the disclosure specifically target CLK kinases. In some embodiments, the compounds described herein can target CLK kinase activity to treat diseases, such as cancers, through modulation of pre-mRNA splicing via inhibition of CLK kinase activity. In some embodiments, methods of treating a target cancer, such as myelodysplastic syndromes (MDS), chronic myelomonocytic leukemia, AML, lung cancer, breast cancer, and pancreatic cancer are described.

In some embodiments, compounds as described herein can be useful in connection with the treatment of diseases, such as cancer, such as AML, by inhibiting one or more of aberrant FLT3, including oncogenic driver mutations such as FLT3-ITD and FLT3 resistance mutations, such as resistance mutations in the activating loop residues (e.g., D835, I836, D839, and Y842), or in the gatekeeper residue F691 of FLT3, aberrant PIM kinases, and/or aberrant CLK kinases.

In the inhibitory methods of the disclosure, an “effective amount” means an amount sufficient to inhibit the target protein. Measuring such target modulation may be performed by routine analytical methods such as those described below. Such modulation is useful in a variety of settings, including in vitro assays. In such methods, the cell is preferably a cancer cell with abnormal signaling due to a mutation of FLT3, PIM, and/or CLK as described herein.

In treatment methods according to the disclosure, an “effective amount” means an amount or dose sufficient to generally bring about the desired therapeutic benefit in subjects needing such treatment, such as those described herein having a disease, such as cancer, such as AML, including those associated with aberrant FLT3, including oncogenic driver mutations such as FLT3-ITD and FLT3 resistance mutations, such as resistance mutations in the activating loop residues (e.g., D835, 1836, D839, and Y842), or in the gatekeeper residue F691 of FLT3, aberrant PIM kinases, and/or aberrant CLK kinases. Effective amounts or doses of the compounds of the disclosure may be ascertained by routine methods, such as modeling, dose escalation, or clinical trials, taking into account routine factors, e.g., the mode or route of administration or drug delivery, the pharmacokinetics of the agent, the severity and course of the infection, the subject's health status, condition, and weight, and the judgment of the treating physician. An exemplary dose is in the range of about from about 0.1 mg to 1 g daily, or about 1 mg to 50 mg daily, or about 50 to 250 mg daily, or about 250 mg to 1 g daily. The total dosage may be given in single or divided dosage units (e.g., BID, TID, QID).

Once improvement of the patient's disease has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms. Patients may also require chronic treatment on a long-term basis.

### Drug Combinations

The inventive compounds described herein may be used in pharmaceutical compositions or methods in combination with one or more additional active ingredients in the treatment of the diseases and disorders described herein. Further additional active ingredients include other therapeutics or agents that mitigate adverse effects of therapies for the intended disease targets. Such combinations may serve to increase efficacy, ameliorate other disease symptoms, decrease one or more side effects, or decrease the required dose of an inventive compound. The additional active ingredients may be administered in a separate pharmaceutical composition from a compound of the present disclosure or may be included with a compound of the present disclosure in a single pharmaceutical composition. The additional active ingredients may be administered simultaneously with, prior to, or after administration of a compound of the present disclosure.

Combination agents include additional active ingredients are those that are known or discovered to be effective in treating the diseases and disorders described herein, including those active against another target associated with the disease. For example, compositions and formulations of the disclosure, as well as methods of treatment, can further comprise other drugs or pharmaceuticals, e.g., other active agents useful for treating or palliative for the target diseases or related symptoms or conditions. For cancer indications, additional such agents include, but are not limited to, kinase inhibitors, such as ALK inhibitors (e.g., crizotinib), Raf inhibitors (e.g., vemurafenib), VEGFR inhibitors (e.g., sunitinib), standard chemotherapy agents such as alkylating agents, antimetabolites, anti-tumor antibiotics, topoisomerase inhibitors, platinum drugs, mitotic inhibitors, antibodies, hormone therapies, or corticosteroids.

### Chemical Synthesis Methods

The following examples are offered to illustrate but not to limit the disclosure. One of skill in the art will recognize that the following synthetic reactions and schemes may be modified by choice of suitable starting materials and reagents in order to access other compounds of Formula (I)-(VI).

Abbreviations: The examples described herein use materials, including but not limited to, those described by the following abbreviations known to those skilled in the art:

| | |
|---|---|
| g | grams |
| eq | equivalents |
| mmol | millimoles |
| mL | milliliters |
| EtOAc | ethyl acetate |
| MHz | megahertz |
| ppm | parts per million |
| δ | chemical shift |
| s | singlet |
| d | doublet |
| t | triplet |
| q | quartet |
| quin | quintet |
| br | broad |
| m | multiplet |
| Hz | hertz |
| THF | tetrahydrofuran |
| ° C. | degrees Celsius |
| PE | petroleum ether |
| EA | ethyl acetate |
| R_{f} | retardation factor |
| N | normal |
| J | coupling constant |
| DMSO-d₆ | deuterated dimethyl sulfoxide |
| n-BuOH | n-butanol |
| DIEA | n,n-diisopropylethylamine |
| TMSCl | trimethylsilyl chloride |
| min | minutes |
| hr | hours |
| Me | methyl |
| Et | ethyl |
| i-Pr | isopropyl |
| TLC | thin layer chromatography |
| M | molar |
| Compd# | compound number |
| MS | mass spectrum |
| m/z | mass-to-charge ratio |
| Ms | methanesulfonyl |
| FDPP | pentafluorophenyl diphenylphosphinate |
| Boc | tert-butyloxycarbonyl |
| TFA | trifluoroacetic acid |
| Tos | toluenesulfonyl |
| DMAP | 4-(dimethylamino)pyridine |
| mM | micromolar |
| ATP | adenosine triphosphate |
| IC₅₀ | half maximal inhibitory concentration |
| U/mL | units of activity per milliliter |
| KHMDS | potassium bis(trimethylsilyl)amide |
| DIAD | diisopropyl azodicarboxylate |
| MeTHF | 2-methyltetrahydrofuran |
| MOM | methoxymethyl |
| DCM | dichloromethane |
| DMF | N,N-dimethylformamide |
| DPPA | diphenyl phosphoryl azide |
| DBU | 1,8-diazabicyclo[5.4.0]undec-7-ene |
| DIPEA | N,N-diisopropylethylamine |
| SEM | [2-(Trimethylsilyl)ethoxy]methyl acetal |
| Hex | hexanes |
| Pd(dppf)Cl₂ | [1,1′-Bis(diphenylphosphino)ferrocene]dichloropalladium(II) |
| MeCN (ACN) | Acetonitrile |
| Pd₂(dba)₃ | Tris(dibenzylideneacetone)dipalladium(0) |
| Hunig's Base | N,N-diisopropylethylamine |
| TBAF | Tert butyl ammonium fluoride |
| PPh₃ | Triphenyl phosphine |
| RT | Room Temperature |
| p-TSA | Para-Tolylsulfonic acid |
| t-BuOH | Tert-Butanol |
| Pd(amphos)Cl₂ | Dichlorobis[di-tert-butyl(4- |
| | dimethylaminophenyl)phosphine]palladium(II) |
| mCPBA | Meta-Chloroperoxy benzoic acid |
| AcOH | Acetic Acid |
| DMAc | N,N-Dimethylformamide |
| BPD | 4,4,5,5-tetramethyl-2-(4,4,5,5-tetramethyl-1,3,2- |
| | dioxaborolan-2-yl)-1,3,2-dioxaborolane |
| MTBE | Methy tert-Butyl Ether |
| NBS | N-bromosuccinimide |
| NIS | N-iodosuccinimide |
| T₃P | Propylphosphonic anhydride |
| HATU | 1-[Bis(dimethylamino)methylene]-1H-1,2,3-triazolo[4,5- |
| | b]pyridinium 3-oxid hexafluorophosphate |
| B₂pin₂ | Bis(pinacolato)diboron |

The proposed targets can be prepared via the conventional chemistry or following the general schemes as shown below which use a selected example for illustration:

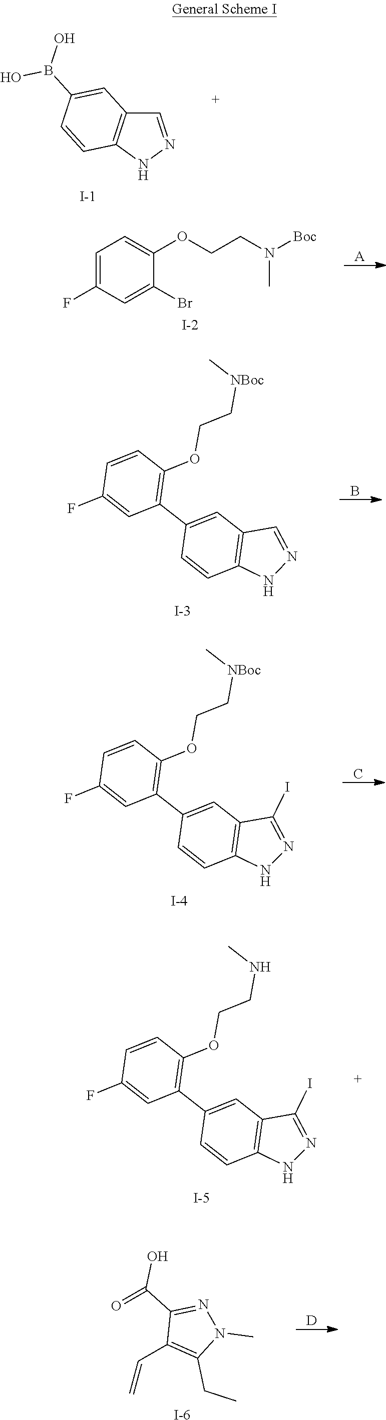

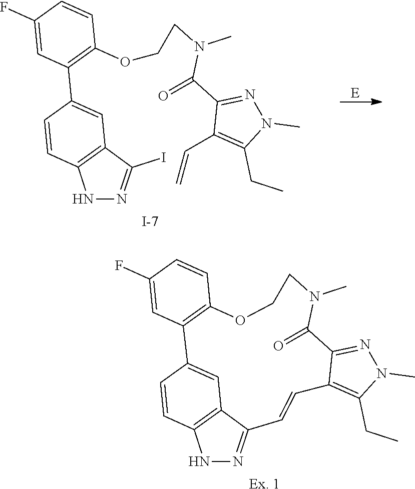

General Scheme I is using Example 1 as an illustration. Compounds I-1 and I-2 are prepared via conventional chemistry from commercially available materials. Under palladium catalyzed coupling condition A, compounds I-1 and 1-2 are converted to a product, 1-3, which then reacted with iodine under condition B to generate I-4, de-Boc under condition C to generate I-5, and amide coupling with a variety of carboxylic acid I-6 under condition D to generate 1-7. Under palladium-catalyzed Heck coupling condition E 1-7 is macrocyclized to the final product, e.g., Ex. 1.

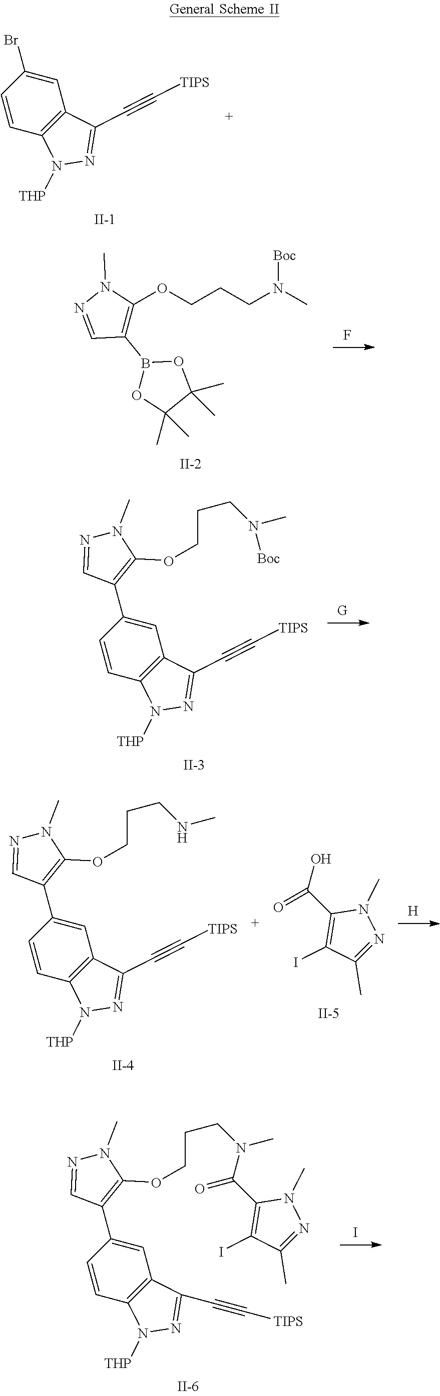

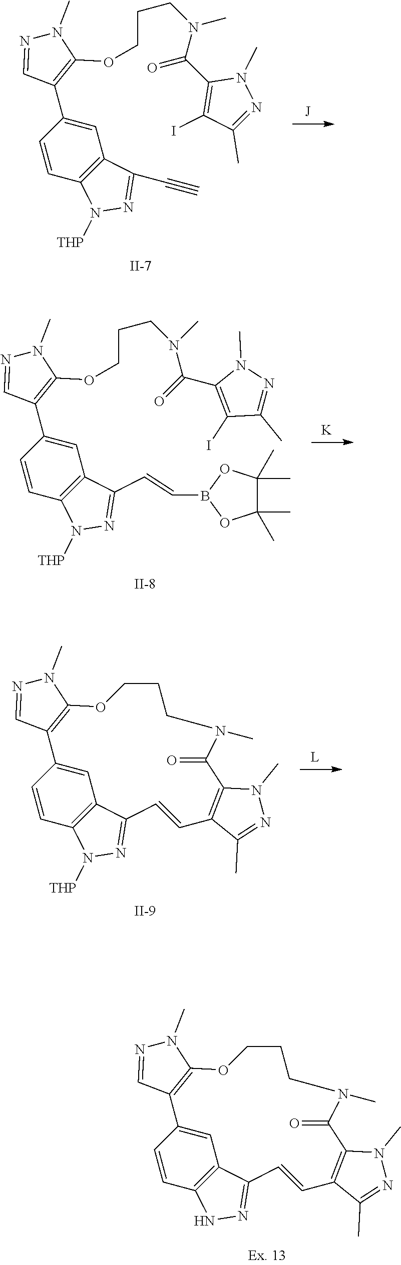

General scheme II is using Example 13 as an illustration. Compound II-1 and II-2 are prepared via conventional chemistry from commercially available materials. Under palladium catalyzed coupling condition F, compounds II-1 and II-2 are converted to a product, II-3, which then de-Boc under condition G to generate II-4 and followed by amide coupling with a variety of carboxylic acid, e.g. II-5 under condition H to generate II-6. II-6 is deprotected under condition I to provide II-7, which is converted to a boronic ester II-8 under condition J. Under palladium-catalyzed Suzuki coupling condition II-8 is macrocyclized to generate II-9. After deprotection under condition L, II-9 is converted to the final product, e.g., Ex. 13.

### General Scheme III

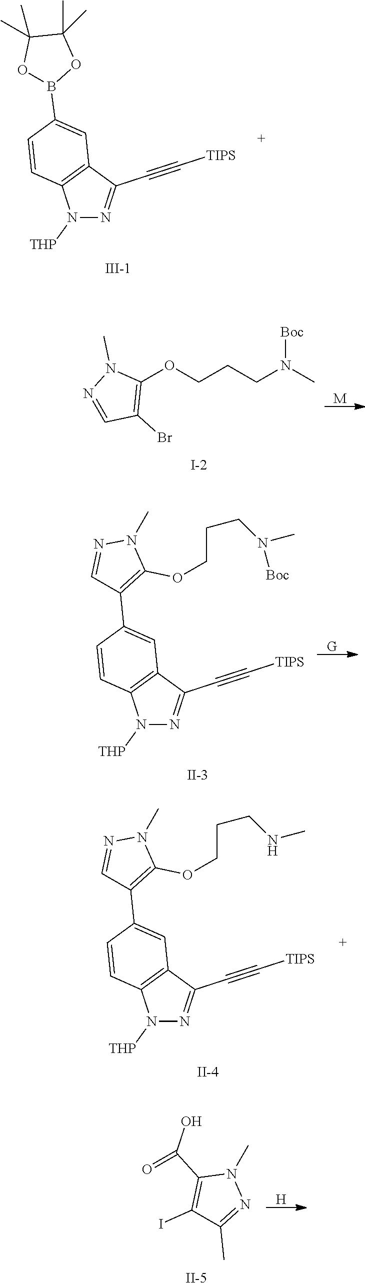

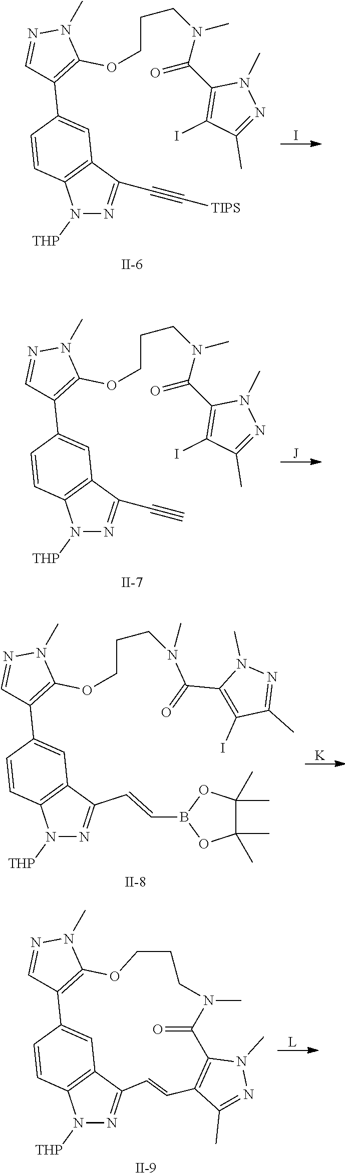

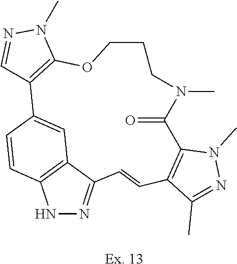 [SMILES: Cc1nn(C)c2c1/C=C/c1n[nH]c3ccc(cc13)-c1cnn(C)c1OCCCN(C)C2=O ; "Ex. 13"]

The general scheme III is using Example 13 as an illustration. The boronic ester III-1 and bromo-starting material III-3 are exchanged in comparison with Compound II-1 and II-2. Under palladium catalyzed coupling condition M, compounds III-1 and III-2 are converted to the same product as that in General Scheme II, II-3, which then de-Boc under condition G to generate II-4 and followed by amide coupling with a variety of carboxylic acid, e.g. II-5 under condition H to generate II-6. II-6 is deprotected under condition I to provide II-7, which is converted to a boronic ester II-8 under condition J. Under palladium-catalyzed Suzuki coupling condition II-8 is macrocyclized to generate II-9. After deprotection under condition L, II-9 is converted to the final product, e.g., Ex. 13.

### Preparation of tert-butyl N-[2-(2-bromo-4-fluoro-phenoxy) ethyl]-N-methyl-carbamate (II-2-1)

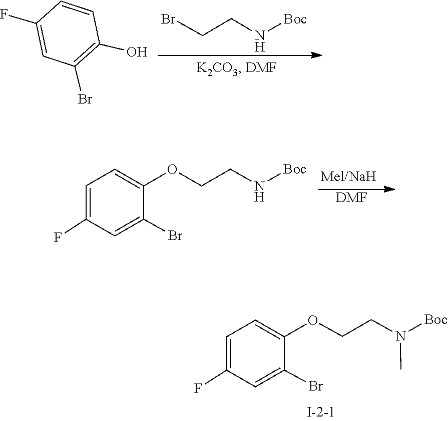

Step 1. To a solution of 2-bromo-4-fluoro-phenol (5.00 g, 26.2 mmol, 1 eq) in DMF (120 mL) was added K₂CO₃ (10.8 g, 78.5 mmol, 3 eq) and tert-butyl N-(2-bromoethyl) carbamate (7.04 g, 31.4 mmol, 1.2 eq). The mixture was stirred at 80° C. for 2 hours. LCMS showed starting material was consumed completely and desired MS in main peak. The mixture was diluted with water (300 mL) and extracted with EtOAc (50 mL*4). The combined organic layer was dried over anhydrous Na₂SO₄, filtered and the filtrate was concentrated in vacuum to give tert-butyl N-[2-(2-bromo-4-fluoro-phenoxy) ethyl]carbamate (9.45 g, crude) was obtained as light yellow oil. ¹H NMR (400 MHz, CDCl₃) δ=7.30 (dd, J=8.0, 3.2 Hz, 1H), 6.94-7.02 (m, 1H), 6.82-6.89 (m, 1H), 5.07 (s, 1H), 4.05 (t, J=4.8 Hz, 2H), 3.57 (q, J=10.4, 5.2 Hz, 2H), 1.46 (s, 11H).

Step 2. To a solution of tert-butyl N-[2-(2-bromo-4-fluoro-phenoxy)ethyl]carbamate (7.50 g, 22.4 mmol, 1 eq) in DMF (80 mL) was added NaH (1.35 g, 33.7 mmol, 60% purity, 1.5 eq) at 0° C. and stirred for 30 minutes. Then Mel (3.82 g, 26.9 mmol, 1.2 eq) was added to the mixture and stirred at 15° C. for 3 hours. The mixture was quenched by water (150 mL) and extracted with EtOAc (50 mL*4). The combined organic layer was dried over anhydrous Na₂SO₄, filtered and the filtrate was concentrated in vacuum to give I-2-1 (7.80 g, 22.4 mmol, 99.8% yield) was obtained as yellow solid.

### Preparation of tert-butyl N-[2-(2-bromo-4-fluoro-phenoxy) ethyl]-N-methyl-carbamate (I-2-2)

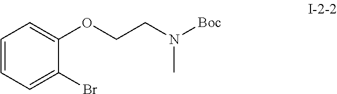 [SMILES: CN(CCOc1ccccc1Br)C(=O)OC(C)(C)C]

I-2-2 was prepared following similar procedures as I-2-1 using 2-bromo-phenol as starting material.

### Preparation of tert-butyl N-[2-(4-bromo-2-methyl-pyrazol-3-yl)oxyethyl]-N-methyl-carbamate (I-2-3)

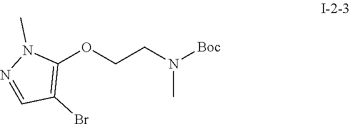 [SMILES: CN(CCOc1c(Br)cnn1C)C(=O)OC(C)(C)C]

I-2-3 was prepared first following similar procedures as I-2-1 using 2-methylpyrazol-3-ol as starting material. Then the bromo-group was introduced. To a solution of tert-butyl N-methyl-N-[2-(2-methylpyrazol-3-yl)oxyethyl]carbamate (2 g, 7.83 mmol, 1 eq) in ACN (20 mL) was added NBS (1.44 g, 8.07 mmol, 1.03 eq). The mixture was stirred at 25° C. for 2 hr. On completion, the mixture was concentrated and the residue was purified by silica gel column chromatography to provide I-2-3 (1.73 g, 5.18 mmol, 66.08% yield) as red oil. ¹H NMR (400 MHz, DMSO-d₆) δ=7.32 (s, 1H), 4.24 (t, J=5.6 Hz, 2H), 3.54 (s, 3H), 3.48 (t, J=5.6 Hz, 2H), 2.81 (s, 3H), 1.31 (d, J=4.0 Hz, 9H).

### Preparation of 5-ethyl-1-methyl-4-vinyl-pyrazole-3-carboxylic acid (I-6-1) and 5-ethyl-2-methyl-4-vinyl-pyrazole-3-carboxylic acid (I-6-2)

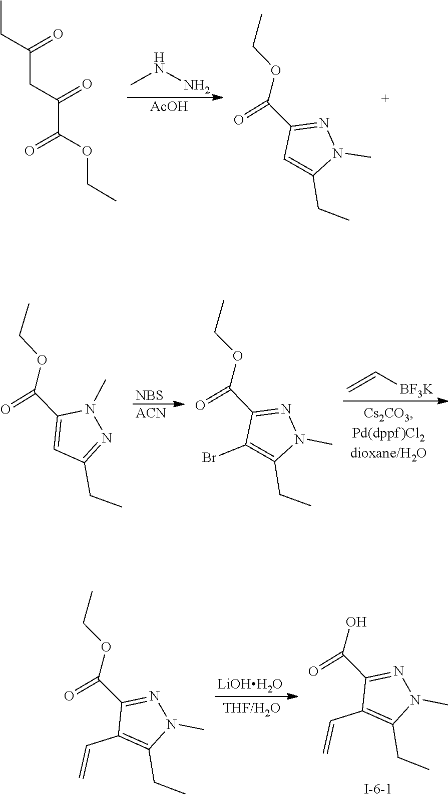

Step 1. To a solution of ethyl 2, 4-dioxohexanoate (10.0 g, 58.1 mmol, 1 eq) in AcOH (65.7 g, 1.09 mol, 18.8 eq) was added methylhydrazine (7.45 g, 64.7 mmol, 40% purity, 1.11 eq) at 0° C. The mixture was stirred at 15° C. for 5 hours and concentrated in vacuum. The residue was purified by combi flash chromatography (120 g silica gel column, EtOAc in PE from 0% to 50%) to provide ethyl 5-ethyl-1-methyl-pyrazole-3-carboxylate (10.1 g, 55.5 mmol, 95.5% yield) as yellow oil. ¹H NMR (400 MHz, CDCl₃) δ=6.59 (s, 1H), 4.39 (q, J=14.4, 7.2 Hz, 2H), 3.85 (s, 3H), 2.62 (q, J=14.4, 7.2 Hz, 2H), 1.39 (t, J=7.2 Hz, 3H), 1.28 (t, J=7.6 Hz, 3H).

Ethyl 5-ethyl-2-methyl-pyrazole-3-carboxylate (1.33 g, 7.30 mmol, 12.6% yield) was obtained as colorless oil. ¹H NMR (400 MHz, CDCl₃) δ=6.65 (s, 1H), 4.34 (q, J=7.2 Hz, 2H), 4.18-4.11 (m, 4H), 2.65 (q, J=15.2, 7.6 Hz, 2H), 1.38 (t, J=7.2 Hz, 3H), 1.25 (t, J=7.6 Hz, 3H).

Step 2. To a solution of ethyl 5-ethyl-1-methyl-pyrazole-3-carboxylate (10.0 g, 54.9 mmol, 1 eq) in MeCN (200 mL) was added NBS (10.7 g, 60.4 mmol, 1.1 eq). The mixture was stirred at 15° C. for 3 hours. The mixture was diluted with water (200 mL) and extracted with EtOAc (50 mL*3). The combined organic layer was dried over anhydrous Na₂SO₄, filtered and the filtrate was concentrated in vacuum to give crude ethyl 4-bromo-5-ethyl-1-methyl-pyrazole-3-carboxylate (13.4 g, 51.4 mmol, 93.8% yield) as yellow oil. ¹H NMR (400 MHz, CDCl₃) δ=4.41 (q, J=7.2 Hz, 2H), 3.91 (s, 3H), 2.78-2.64 (m, 2H), 1.40 (t, J=7.2 Hz, 3H), 1.18 (t, J=7.6 Hz, 3H).

Step 3. To a solution of ethyl 4-bromo-5-ethyl-1-methyl-pyrazole-3-carboxylate (13.4 g, 51.5 mmol, 1 eq), potassium hydride; trifluoro (vinyl) boron (13.8 g, 103 mmol, 2 eq), Cs₂CO₃ (50.3 g, 154 mmol, 3 eq), Pd(dppf)Cl₂ (3.77 g, 5.15 mmol, 0.1 eq) in dioxane (200 mL) and H₂O (40 mL) was stirred at 80° C. under N₂ for 3 hours. The mixture was stirred at 80° C. for 16 hour and cooled to ambient temperature. The mixture was separated, and the organic layer was concentrated in vacuum. The residue was purified by combi flash chromatography (120 g silica gel column, EtOAc in PE from 0% to 60%) to provide ethyl 5-ethyl-1-methyl-4-vinyl-pyrazole-3-carboxylate (7.62 g, 36.6 mmol, 71.1% yield) as brown oil. ¹H NMR (400 MHz, CDCl₃) δ=7.05 (dd, J=14.0, 11.6 Hz, 1H), 5.47-5.26 (m, 2H), 4.42 (J=7.2 Hz, 2H), 3.90 (s, 3H), 2.83-2.69 (m, 2H), 1.46-1.37 (m, 3H), 1.27-1.17 (m, 3H)

Step 4. To a solution of ethyl 5-ethyl-1-methyl-4-vinyl-pyrazole-3-carboxylate (1.00 g, 4.80 mmol, 1 eq) in THF (5 mL), MeOH (5 mL), H₂O (3 mL) was added LiOH·H₂O (604 mg, 14.4 mmol, 3 eq). The mixture was stirred at 15° C. for 5 hours. LCMS showed desired MS in main peak. The mixture was added 2 N HCl to just pH-5. The result solution was extracted with EtOAc (10 mL*4). The combined organic layer was dried over anhydrous Na₂SO₄, filtered and the filtrate was concentrated in vacuum to give crude. The residue was purified by Prep-HPLC (column: Phenomenex luna C18 150*25 mm*10 um; mobile phase: [water (0.225% FA)-ACN]; B %: 40%-79%, 11 min). 5-ethyl-1-methyl-4-vinyl-pyrazole-3-carboxylic acid (746 mg, 4.14 mmol, 86.2% yield) was obtained as brown solid. ¹H NMR (400 MHz, MeOD-d₄) δ=7.03 (dd, J=18.0, 11.6 Hz, 1H), 5.44 (dd, J=18.0, 1.6 Hz, 1H), 5.27 (dd, J=11.8, 1.6 Hz, 1H), 3.87 (s, 3H), 2.84 (q, J=7.6 Hz, 2H) 1.23 (t, J=7.6 Hz, 3H).

I-6-2 was prepared as white solid using ethyl 5-ethyl-2-methyl-pyrazole-3-carboxylate in step 2. ¹H NMR (400 MHz, MeOD-d₄) δ=7.07 (dd, J=18.0, 11.6 Hz, 1H), 5.44 (dd, J=18.0, 1.6 Hz, 1H), 5.30 (dd, J=11.6, 1.6 Hz, 1H), 4.04 (s, 3H), 2.74 (q, J=7.2 Hz, 2H), 1.25 (t, J=7.6 Hz, 3H).

### Preparation of 1-methyl-4-vinyl-pyrazole-3-carboxylic acid (II-6-3)

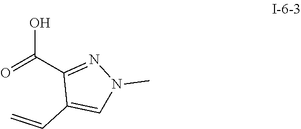

I-6-3 was prepared as yellow solid following similar methods as Step 3 and Step 4 in I-6-1 preparation using methyl 4-bromo-1-methyl-pyrazole-3-carboxylate as starting material. ¹H NMR (400 MHz, DMSO-d₆) δ=12.79-12.48 (m, 1H), 8.09 (s, 1H), 7.02 (dd, J=11.2, 18.0 Hz, 1H), 5.54 (dd, J=1.6, 18.0 Hz, 1H), 5.13 (dd, J=1.6, 11.2 Hz, 1H), 3.87 (s, 3H).

### General Method A: Preparation of (18E)-17-ethyl-7-fluoro-13,16-dimethyl-2,12,13,16-tetrahydro-3,5-ethenodipyrazolo[3,4-f:3′,4′-j][1,4]benzoxazacyclopentadecin-14(11H)-one (Ex. 1)

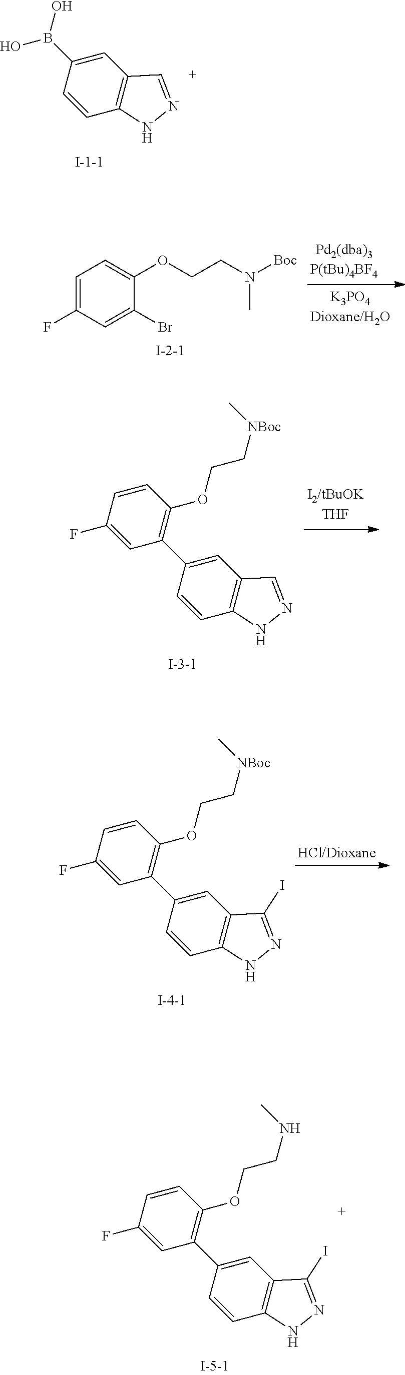 [SMILES: OB(O)c1ccc2[nH]ncc2c1 ; CN(CCOc1ccc(F)cc1Br)C(=O)OC(C)(C)C ; CN(CCOc1ccc(F)cc1-c1ccc2[nH]ncc2c1)C(=O)OC(C)(C)C ; CN(CCOc1ccc(F)cc1-c1ccc2[nH]nc(I)c2c1)C(=O)OC(C)(C)C ; CNCCOc1ccc(F)cc1-c1ccc2[nH]nc(I)c2c1]

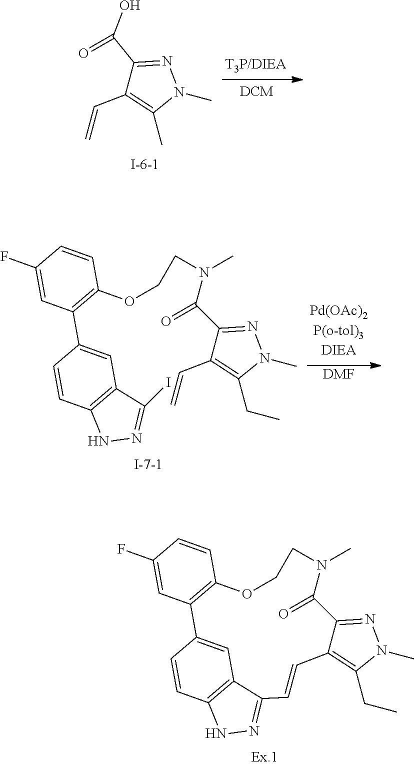

Step 1. To a mixture of I-2-1 (4.00 g, 11.5 mmol, 1 eq) and 5-(4,4,5,5-tetramethyl-1,3,2-dioxaborolan-2-yl)-1H-indazole (3.37 g, 13.8 mmol, 1.2 eq) in dioxane (60 mL) and H₂O (12 mL) was added K₃PO₄ (7.32 g, 34.5 mmol, 3 eq), tritert-butylphosphonium;tetrafluoroborate (333 mg, 1.15 mmol, 0.1 eq), and Pd₂(dba)₃ (526 mg, 0.575 mmol, 0.05 eq). The resulting mixture was stirred at 120° C. under N₂ for 16 hours. The mixture was separated and the organic layer was dried over anhydrous Na₂SO₄ and filtered. The filtrate was concentrated in vacuum and the residue was purified by Combi flash chromatography to provide I-3-1 (2.44 g, 6.33 mmol, 55.1% yield) as brown oil. ¹H NMR (400 MHz, CDCl₃) δ=8.12 (s, 1H), 7.87 (s, 1H), 7.60-7.47 (m, 2H), 7.10 (d, J=8.8 Hz, 1H), 7.04-6.97 (m, 1H), 6.96-6.89 (m, 1H), 5.03 (d, J=21.2 Hz, 2H), 3.49 (s, 2H), 2.70 (d, J=18 Hz, 2H), 1.41 (s, 9H).

Step 2. To a solution of I-3-1 (2.44 g, 6.33 mmol, 1 eq) in THF (40 mL) was added t-BuOK (2.13 g, 18.9 mmol, 3 eq). The resulting mixture was stirred at 0° C. for 5 minutes followed by addition of 12 (2.09 g, 8.23 mmol, 1.3 eq) in THF (5 mL) in dropwise method. The resulting mixture was stirred at 25° C. for another 2 hours and the mixture was filtered. The filtrate was concentrated in vacuum and the residue was purified by Combi flash to provide I-4-1 (1.87 g, 3.66 mmol, 57.77% yield) as brown oil. ¹H NMR (400 MHz, CDCl₃) δ=7.63 (s, 2H), 7.52 (d, J=8.8 Hz, 1H), 7.12 (d, J=7.2 Hz, 1H), 7.07-6.99 (m, 1H), 6.97-6.90 (m, 1H), 4.07 (d, J=22.8 Hz, 2H), 3.51 (s, 2H), 2.72 (d, J=15.2 Hz, 3H), 1.42 (s, 9H).

Step 3. To a solution of I-4-1 (1.00 g, 1.96 mmol, 1 eq) in dioxane (8 mL) was added HCl/dioxane (4 M, 8.77 mL, 17.9 eq). The mixture was stirred at 15° C. for 3 hours. The mixture was filtered and the solid was dried in vacuum to give I-5-1 (890 mg, crude) as white solid. ¹H NMR (400 MHz, MeOD-d₄) δ=7.68-7.57 (m, 3H), 7.25-7.06 (m, 3H), 4.24-4.16 (m, 2H), 3.35-3.33 (m, 2H), 2.61 (s, 3H).

Step 4. To a solution of I-5-1 (417 mg, 0.932 mmol, 1.05 eq, HCl salt) and I-6-1 (160 mg, 0.888 mmol, 1 eq), DIEA (574 mg, 4.44 mmol, 5 eq) in DCM (8 mL) was added T₃P (847 mg, 1.33 mmol, 50% purity, 1.5 eq) at 0° C. The mixture was stirred at 15° C. for 2.5 hours, diluted with water (30 mL), and extracted with EtOAc (15 mL*3). The combined organic layers were dried over anhydrous Na₂SO₄, filtered and the filtrate was concentrated in vacuum. The residue was purified by combi flash (40 g silica gel column, EtOAc in PE from 0% to 100%) to provide I-7-1 (147 mg, 0.256 mmol, 28.9% yield) as white solid. LCMS: m/z 574.4 (M+1).

Step 5. To a solution of I-7-1 (147 mg, 0.256 mmol, 1 eq) in DMF (28 mL) was added tris-o-tolylphosphane (7.80 mg, 0.0256 mmol, 0.1 eq), N-ethyl-N-isopropyl-propan-2-amine (66.3 mg, 0.513 mmol, 2 eq) and Pd(OAc)₂ (2.88 mg, 0.128 mmol, 0.05 eq). The resulting mixture was stirred at 120° C. for 12 hours. The mixture was concentrated in vacuum and the residue was purified by prep-HPLC (column: Phenomenex Luna C18 100*30 mm*5 um; mobile phase: [water (0.225% FA)-ACN]; B %: 38%-68%, 10 min) to provide Ex. 1 (5.09 mg, 4.42% yield) as a white solid.

Ex. 2-5 were prepared following General Method A.

### Preparation of 2-(5-bromo-1-tetrahydropyran-2-yl-indazol-3-yl)ethynyl-triisopropyl-silane (II-1-1)

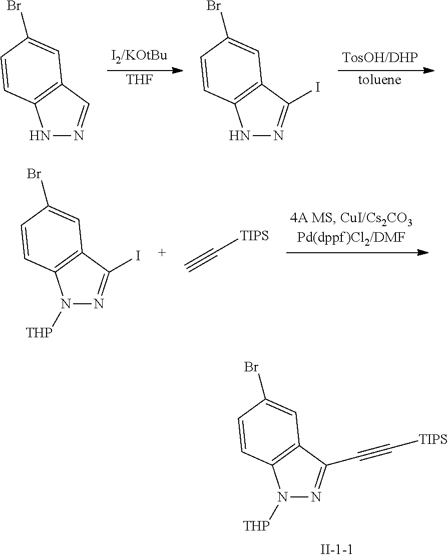

Step 1. A solution of 5-bromo-1H-indazole (21.0 g, 107 mmol, 1 eq) in THE (250 mL) was cooled down on an ice bath and KOtBu (35.9 g, 320 mmol, 3 eq) was added portion wise. The resulting slurry was stirred at 0° C. and a solution of I₂ (54.1 g, 213 mmol, 42.9 mL, 2 eq) in THF (250 mL) was added dropwise. The mixture was stirred at 25° C. for 12 hours. On completion, the reaction mixture was filtered and the filtrate was diluted with H₂O (20 mL) and extracted with EtOAc (20 mL*3). The combined organic layers were washed with brine (20 mL*3), dried over Na₂SO₄, filtered and concentrated under reduced pressure. The residue was purified by flash silica gel chromatography to afford 5-bromo-3-iodo-1H-indazole (120 g, 350 mmol, 82% yield, 94% purity) as a white solid. LCMS: 324.7 (M+1).

Step 2. To a mixture of 5-bromo-3-iodo-1H-indazole (25.0 g, 77.4 mmol, 1 eq) and 3,4-dihydro-2H-pyran (13.0 g, 155 mmol, 2 eq) in toluene (250 mL) was added 4-methylbenzenesulfonic acid (2.67 g, 15.5 mmol, 0.2 eq). The mixture was stirred at 90° C. for 12 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford 5-bromo-3-iodo-1-tetrahydropyran-2-yl-indazole (24.0 g, 58.9 mmol, 76% yield) as a white solid. ¹H NMR (400 MHz, CDCl₃) δ=7.64 (d, J=1.6 Hz, 1H), 7.54-7.44 (m, 2H), 5.68 (dd, J=3.2, 9.1 Hz, 1H), 4.04-3.95 (m, 1H), 3.79-3.66 (m, 1H), 2.58-2.46 (m, 1H), 2.20-2.03 (m. 2H), 1.87-1.54 (m, 3H).

Step 3. To a mixture of 5-bromo-3-iodo-1-tetrahydropyran-2-yl-indazole (23.0 g, 56.5 mmol, 1 eq) and ethynyl(triisopropyl)silane (11.3 g, 62.2 mmol, 1.1 eq) in DMF (250 mL) was added Cs₂CO₃ (55.2 g, 170 mmol, 3 eq), Pd(dppf)Cl₂ (2.48 g, 3.39 mmol, 0.06 eq) and CuI (646 mg, 3.39 mmol, 0.06 eq) under N₂. The mixture was stirred at 25° C. for 3 hours. On completion, the reaction was diluted with H₂O and extracted with EtOAc. The combined organic layers were washed with brine, dried over Na₂SO₄, filtered and concentrated under reduced pressure. The residue was purified by flash silica gel chromatography to afford compound 2-(5-bromo-1-tetrahydropyran-2-yl-indazol-3-yl)ethynyl-triisopropyl-silane (II-1-1, 38.0 g, 79.9 mmol, 70% yield) as a white solid. ¹H NMR (400 MHz, CDCl₃) δ=7.87 (s, 1H), 7.57-7.43 (m, 2H), 5.70 (dd, J=2.4, 9.2 Hz, 1H), 4.02 (bd, J=11.2 Hz, 1H), 3.80-3.65 (m, 1H), 2.59-2.41 (m, 1H), 2.14 (d, J=3.2 Hz, 1H), 2.08 (s, 1H), 1.79-1.70 (m, 2H), 1.67 (s, 1H), 1.22-1.18 (m, 18H), 1.18-1.14 (m, 3H).

### Preparation of tert-butyl N-methyl-N-[3-[2-methyl-4-(4,4,5,5-tetramethyl-1,3,2-dioxaborolan-2-yl)pyrazol-3-yl]oxyethyl]carbamate (II-2-1)

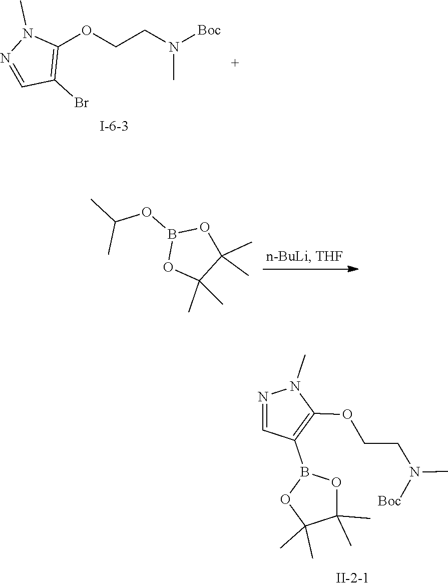 [SMILES: CN(CCOc1c(Br)cnn1C)C(=O)OC(C)(C)C ; CC(C)OB1OC(C)(C)C(C)(C)O1 ; CN(CCOc1c(B2OC(C)(C)C(C)(C)O2)cnn1C)C(=O)OC(C)(C)C]

To a solution of tert-butyl N-[2-(4-bromo-2-methyl-pyrazol-3-yl)oxyethyl]-N-methyl-carbamate (20.0 g, 59.8 mmol, 1 eq) in THF (200 mL) was added n-BuLi (2.5 M, 23.9 mL, 1 eq) at −78° C., the mixture was stirred at this temperature for 30 mins followed by addition of 2-isopropoxy-4,4,5,5-tetramethyl-1,3,2-dioxaborolane (22.2 g, 119 mmol, 2 eq) dropwise at −78° C. The mixture was stirred at −78° C. for 2 hr. On completion, the mixture was quenched with water (200 mL) and extracted with ethyl acetate (250 mL×3). The combined organic phase was dried over anhydrous sodium sulfate, filtered and concentrated to give a residue. The residue was purified by column chromatography to give I1-2-1 (21.2 g, 55.6 mmol, 92% yield) as a yellow oil. LCMS: m/z 381.9 (M+1).

### Preparation of tert-butyl N-[3-[2-methyl-4-(4,4,5,5-tetramethyl-1,3,2-dioxaborolan-2-yl)pyrazol-3-yl]oxyethyl]carbamate (II-2-2)

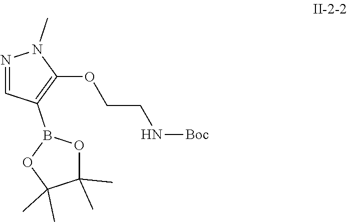 [SMILES: Cn1ncc(B2OC(C)(C)C(C)(C)O2)c1OCCNC(=O)OC(C)(C)C]

II-2-2 was prepared following similar methods as II-2-1.

### Preparation of tert-butyl N-methyl-N-[4-[2-methyl-4-(4,4,5,5-tetramethyl-1,3,2-dioxaborolan-2-yl)pyrazol-3-yl]oxybutyl]carbamate (II-2-3)

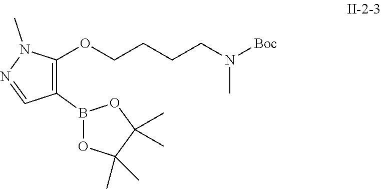 [SMILES: CN(CCCCOc1c(B2OC(C)(C)C(C)(C)O2)cnn1C)C(=O)OC(C)(C)C]

II-2-3 was prepared following similar methods as II-2-1.

### Preparation of tert-butyl N-methyl-N-[4-[2-methyl-4-(4,4,5,5-tetramethyl-1,3,2-dioxaborolan-2-yl)pyrazol-3-yl]oxypropyl]carbamate (II-2-4)

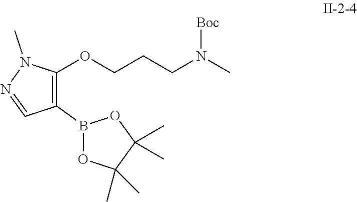 [SMILES: CN(CCCOc1c(B2OC(C)(C)C(C)(C)O2)cnn1C)C(=O)OC(C)(C)C]

II-2-4 was prepared following similar methods as II-2-1.

### Preparation of Tert-butyl N-methyl-N-[2-[2-[2-methyl-4-(4,4,5,5-tetramethyl-1,3,2-dioxaborolan-2-yl)pyrazol-3-yl]oxyethoxy]ethyl]carbamate (II-2-5)

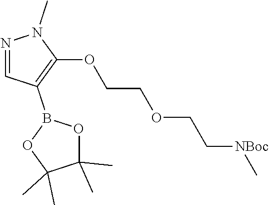 [SMILES: CN(CCOCCOc1c(B2OC(C)(C)C(C)(C)O2)cnn1C)C(=O)OC(C)(C)C]

II-2-5 was prepared following similar methods as II-2-1.

### Preparation of 5-ethyl-4-iodo-2-methyl-pyrazole-3-carboxylic acid (II-5-1)

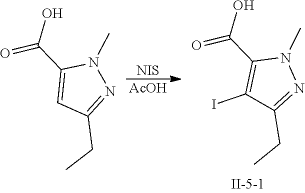

To a solution of 5-ethyl-2-methyl-pyrazole-3-carboxylic acid (1 g, 6.49 mmol, 1 eq) in AcOH (10 mL) was added NIS (1.75 g, 7.78 mmol, 1.2 eq). The mixture was stirred at 90° C. for 2 hours. Additional NIS (291.87 mg, 1.30 mmol, 0.2 eq) was added to the mixture and the result mixture was stirred at 90° C. for 12 hours. The reaction mixture was quenched by addition sat.aq. Na₂SO₃ (20 mL) at 0° C., and then diluted with H₂O (10 mL), K₂CO₃ (20 mg) was added to the mixture, then adjust pH=5 with HCl (2 N) and extracted with EtOAc (10 mL×2). The combined organic layers were washed with brine (10 mL), dried over anhydrous sodium sulfate, filtered and concentrated under reduced pressure to give a residue, which was triturated with Petroleum ether:Ethyl acetate=10:1 to provide II-5-1 (705 mg, 2.41 mmol, 37% yield) as a light yellow solid. LCMS: m/z 280.9 (M+1).

### Preparation of 5-ethyl-4-iodo-1-methyl-pyrazole-3-carboxylic acid (II-5-2), 5-methyl-4-iodo-2-methyl-pyrazole-3-carboxylic acid (II-5-3), 5-methyl-4-iodo-1-methyl-pyrazole-3-carboxylic acid (II-5-4), and 2-ethyl-4-iodo-5-methyl-pyrazole-3-carboxylic acid (II-5-5)

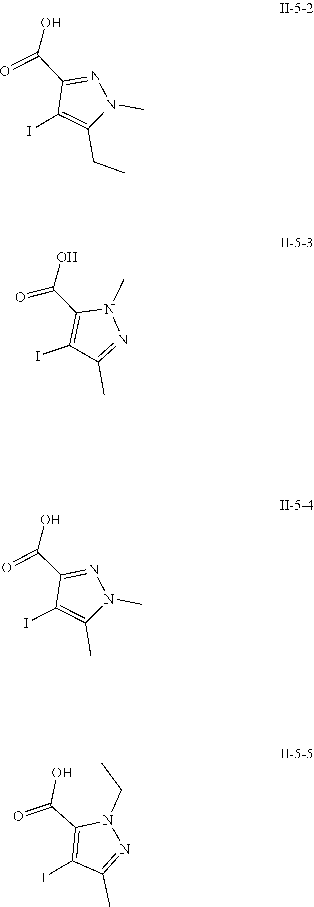

II-5-2, II-5-3, II-5-4 and II-5-5 were prepared following similar methods as II-5-1 using the corresponding pyrazole-3-carboxylic acid as starting materials.

### Preparation of 1-(2-ethoxy-2-oxo-ethyl)-4-iodo-5-methyl-pyrazole-3-carboxylic acid (II-5-5) and 2-(2-ethoxy-2-oxo-ethyl)-4-iodo-5-methyl-pyrazole-3-carboxylic acid (II-5-5)

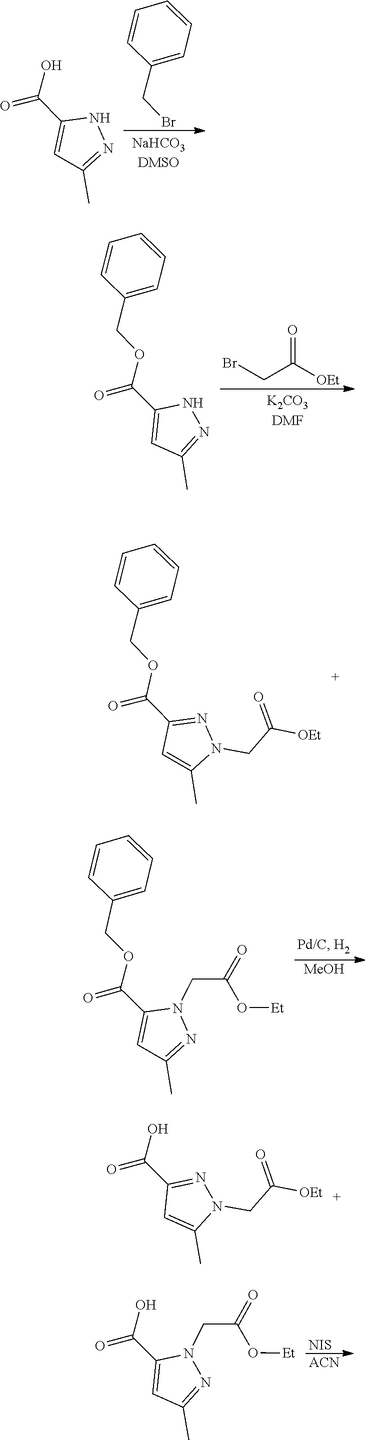

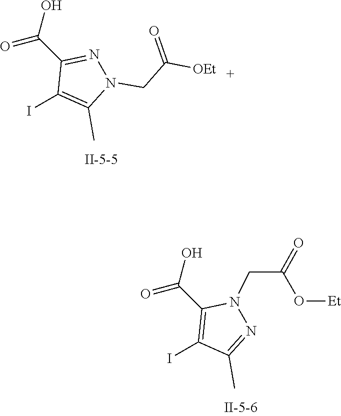 [SMILES: CCOC(=O)Cn1nc(C(=O)O)c(I)c1C ; CCOC(=O)Cn1nc(C)c(I)c1C(=O)O]

Step 1. To a solution of 3-methyl-1H-pyrazole-5-carboxylic acid (10.0 g, 79.3 mmol, 1.0 eq) in DMSO (200 mL) was added NaHCO₃ (7.99 g, 95.2 mmol, 3.70 mL, 1.2 eq). The mixture was stirred at 20° C. for 0.5 hr followed by addition of bromomethylbenzene (13.6 g, 79.3 mmol, 1.0 eq) was added. The mixture was stirred at 20° C. for 4 hr, quenched with water (100 mL), and extracted with ethyl acetate (250 mL×3). The combined organic phase was dried over anhydrous sodium sulfate, filtered and concentrated. The residue was purified by column chromatography to give benzyl 3-methyl-1H-pyrazole-5-carboxylate (3.20 g, 14.1 mmol, 17.7% yield, 95% purity) as a white solid. LCMS: m/z (M+1).

Step 2. To a solution of benzyl 3-methyl-1H-pyrazole-5-carboxylate (3.00 g, 13.9 mmol, 1.0 eq) in DMF (30 mL) were added K₂CO₃ (5.75 g, 41.6 mmol, 3.0 eq) and ethyl 2-bromoacetate (3.48 g, 20.8 mmol, 1.5 eq). The mixture was stirred at 25° C. for 16 hr, quenched with water (200 ml) and extracted with ethyl acetate (50 mL×3). The combined organic phase was dried over anhydrous sodium sulfate, filtered and concentrated. The residue was purified by column chromatography to give benzyl 2-(2-ethoxy-2-oxo-ethyl)-5-methyl-pyrazole-3-carboxylate (850 mg, 2.81 mmol, 20.3% yield) as a colorless oil from the first fraction. ¹H NMR (400 MHz, CDCl₃) δ=7.38-7.35 (m, 4H), 7.34-7.33 (m, 1H), 6.70 (s, 1H), 5.26 (s, 2H), 5.22 (s, 2H), 4.22-4.21 (m, 2H), 2.26 (s, 3H), 1.23-1.19 (m, 3H).

The second fraction provided benzyl 1-(2-ethoxy-2-oxoethyl)-5-methyl-1H-pyrazole-3-carboxylate (2.52 g, 8.34 mmol, 60.1% yield) as a white solid. ¹H NMR (400 MHz, CDCl₃) δ=7.44 (d, J=6.4 Hz, 2H), 7.39-7.30 (m, 3H), 6.63 (s, 1H), 5.37 (s, 2H), 4.93 (s, 2H), 4.23 (q, J=7.2 Hz, 2H), 2.27 (s, 3H), 1.27 (t, J=7.2 Hz, 3H).

Step 3. To a solution of benzyl 1-(2-ethoxy-2-oxoethyl)-5-methyl-1H-pyrazole-3-carboxylate (2.50 g, 8.27 mmol, 1.0 eq) in MeOH (40 mL) was added Pd/C (300 mg, 0.331 mmol, 10% purity, 1.0 eq) under N₂. The suspension was degassed under vacuum and purged with H₂ several times. The mixture was stirred under H₂ (15 psi) at 20° C. for 16 hr. The reaction mixture was filtered and the filtrate was concentrated to give 1-(2-ethoxy-2-oxoethyl)-5-methyl-1H-pyrazole-3-carboxylic acid (1.9 g, crude) as a colorless oil. ¹H NMR (400 MHz, DMSO-d₆) δ=6.49 (s, 1H), 5.10 (s, 2H), 4.17 (q, J=7.2 Hz, 2H), 2.22 (s, 3H), 1.21 (t, J=7.2 Hz, 3H).

Step 4. To a solution of 1-(2-ethoxy-2-oxo-ethyl)-5-methyl-pyrazole-3-carboxylic acid (1.42 g, 6.69 mmol, 1.0 eq) in ACN (20 ml) was added NIS (1.66 g, 7.36 mmol, 1.1 eq). The mixture was stirred at 60° C. for 16 hr. The mixture was dried over anhydrous sodium sulfate, filtered, and concentrated. The residue was purified by column chromatography to give 1-(2-ethoxy-2-oxo-ethyl)-4-iodo-5-methyl-pyrazole-3-carboxylic acid (II-5-5, 1.6 g, 4.26 mmol, 63.7% yield) as a yellow solid. ¹H NMR (400 MHz, DMSO-d₆) δ=12.79-9.38 (m, 1H), 5.23 (s, 211), 4.17 (q, J=7.2 Hz, 2H), 4.03 (q, J=7.2 Hz, 2H), 2.26 (s, 3H), 1.17 (t, J=7.2 Hz, 3H)

II-5-6 was prepared from benzyl 2-(2-ethoxy-2-oxo-ethyl)-5-methyl-pyrazole-3-carboxylate from Step 2 following Step 3 and 4 as II-5-5.

General Method B: Preparation of (17E)-16-ethyl-8,12,14-trimethyl-2,11,12,14-tetrahydro-8H-3,5-ethenotripyrazolo[3,4-f:3′,4′-j:4″,3″-n][1,4]oxazacyclopentadecin-13(10H)-one (Ex. 6)

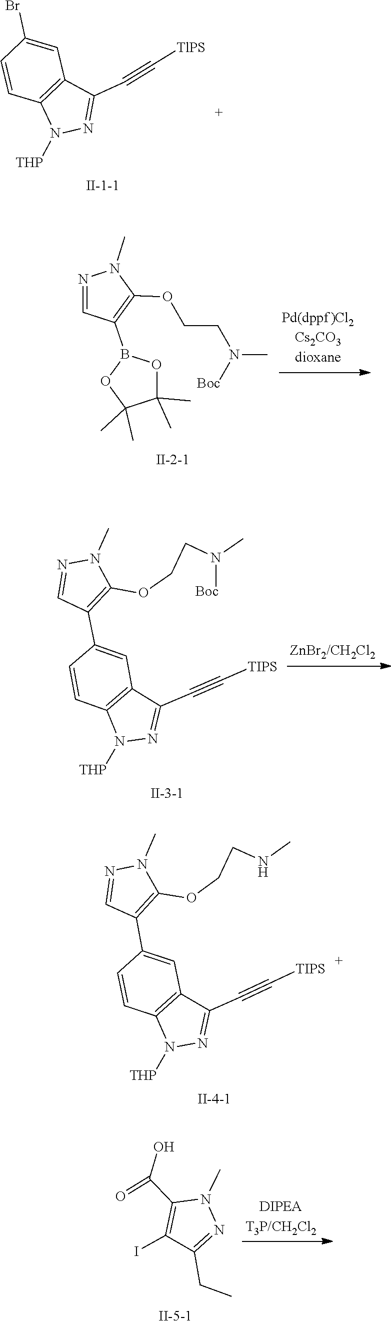

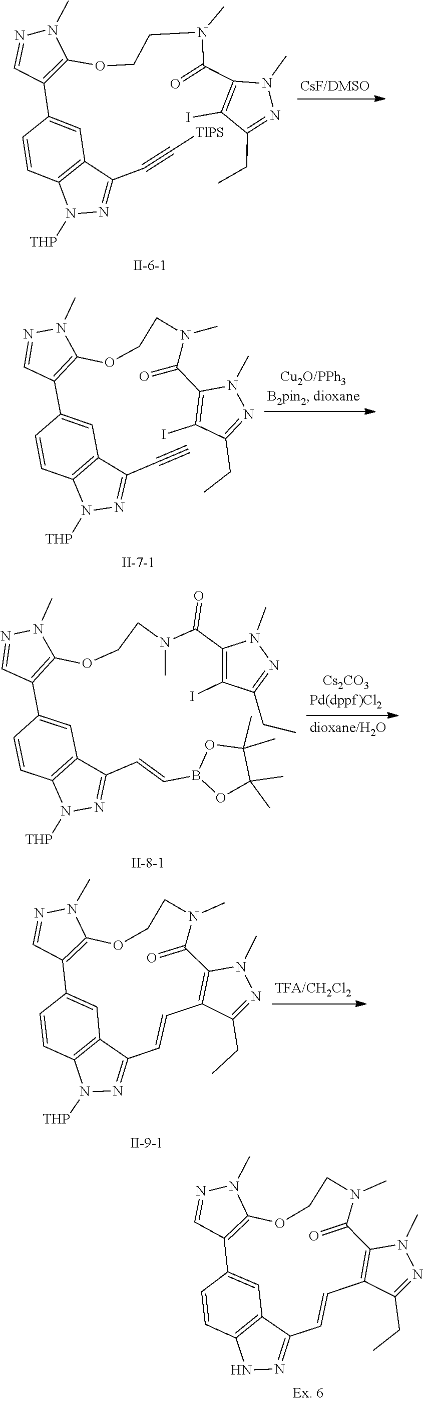

Step 1. A mixture of 2-(5-bromo-1-tetrahydropyran-2-yl-indazol-3-yl)ethynyl-triisopropyl-silane (II-1-1, 5.00 g, 10.83 mmol, 1 eq), tert-butyl N-methyl-N-[2-[2-methyl-4-(4,4,5,5-tetramethyl-1,3,2-dioxaborolan-2-yl)pyrazol-3-yl]oxyethyl]carbamate (II-2-1, 4.96 g, 13.00 mmol, 1.2 eq), Pd(dpp)Cl₂ (1.59 g, 2.17 mmol, 0.2 eq), Cs₂CO₃ (2 M, 16.25 mL, 3 eq) in dioxane (50 mL) was degassed and purged with N₂ for 3 times, and then the mixture was stirred at 80° C. for 5 hours under N₂ atmosphere. The reaction mixture was concentrated under reduced pressure to remove solvent. The residue was diluted with H₂O (50 mL) and extracted with EtOAc (60 mL×3). The combined organic layers were washed with brine (50 mL×3), dried over anhydrous sodium sulfate, filtered and concentrated under reduced pressure to give a residue, which was purified by column chromatography to provide I1-3-1 (5.00 g, 7.86 mmol, 72.6% yield) as a yellow oil. ¹H NMR (400 MHz, CDCl₃) δ=7.82 (s, 1H), 7.65-7.55 (m, 3H), 5.72 (dd, J=2.8, 9.2 Hz, 1H), 4.00 (s, 2H), 3.76 (s, 3H), 3.56-3.47 (m, 2H), 2.99-2.93 (m, 3H), 2.62-2.48 (m, 1H), 2.16 (dd, J=3.6, 8.4 Hz, 1H), 1.83-1.59 (m, 5H), 1.47 (s, 3H), 1.41-1.31 (m, 6H), 1.21-1.18 (m, 21H).

Step 2. To a solution of II-3-1 (1 g, 1.57 mmol, 1 eq) in CH₂Cl₂ (10 mL) was added ZnBr₂ (1.77 g, 7.86 mmol, 5 eq). The mixture was stirred at 25° C. for 2 hours. The reaction mixture was concentrated under reduced pressure to remove solvent. The residue was purified by column chromatography to give II-4-1 (800 mg, 1.49 mmol, 95.0% yield) as a yellow solid. LCMS: m/z 536.1 (M+1).

Step 3. To a solution of II-4-1 (1.5 g, 2.80 mmol, 1 eq) and 5-ethyl-4-iodo-2-methyl-pyrazole-3-carboxylic acid (II-5-1, 705 mg, 2.52 mmol, 0.9 eq) in CH₂Cl₂ (30 mL) were added DIPEA (2.89 g, 22.4 mmol, 8 eq) and T₃P (3.56 g, 5.60 mmol, 50% purity, 2 eq). The mixture was stirred at 40° C. for 12 hours. The reaction mixture was partitioned between H₂O (50 mL) and CH₂Cl₂ (30 mL). The organic phase was separated, washed with brine (30 mL), dried over anhydrous sodium sulfate, filtered and concentrated under reduced pressure to give a residue, which was purified by column chromatography to give II-6-1 (1.15 g, 1.37 mmol, 49.0% yield, 95.1% purity) as a yellow gum.

LCMS: EC5257-79-P1E (M+1: 798.4).

Step 4. To a solution of II-6-1 (1.15 g, 1.44 mmol, 1 eq) in DMSO (12 mL) was added CsF (438 mg, 2.88 mmol, 2 eq). The mixture was stirred at 40° C. for 12 hours. The reaction mixture was partitioned between H₂O (10 mL) and EtOAc (10 mL). The organic phase was separated, washed with brine (10 mL), dried over anhydrous sodium sulfate, filtered and concentrated under reduced pressure to give a residue, which was purified by flash silica gel chromatography to give II-7-1 (650 mg, 1.01 mmol, 70.3% yield) as a yellow oil. ¹H NMR (400 MHz, DMSO-d₆) δ=7.87-7.43 (m, 4H), 5.93-5.85 (m, 1H), 4.55 (d, J=7.6 Hz, 1H), 4.22-4.09 (m, 1H), 3.94-3.61 (m, 8H), 3.58-3.46 (m, 2H), 3.16-3.00 (m, 3H), 2.56-2.52 (m, 3H), 2.40-2.28 (m, 1H), 2.09-1.95 (m, 2H), 1.81-1.68 (m, 1H), 1.65-1.55 (m, 2H), 1.20-1.13 (m, 3H).

Step 5. A mixture of II-7-1 (650 mg, 1.01 mmol, 1 eq), Pin₂B₂(257 mg, 1.01 mmol, 1 eq), PPh₃ (266 mg, 1.01 mmol, 1 eq) and Cu₂O (72.5 mg, 0.507 mmol, 0.5 eq) in dioxane (12 mL) was degassed and purged with N₂ for 3 times, and then the mixture was stirred at 100° C. for 12 hours under N₂ atmosphere. The reaction mixture was partitioned between H₂O (30 mL) and EtOAc (30 mL). The organic phase was separated, washed with brine (20 mL), dried over anhydrous sodium sulfate, filtered and concentrated under reduced pressure to give a residue, which was purified by flash silica gel chromatography to give II-8-1 (750 mg, 0.974 mmol, 96.2% yield) as a yellow solid. LCMS: m/z 770.4 (M+1).

Step 6. A mixture of II-8-1 (700 mg, 0.91 mmol, 1 eq), Cs₂CO₃ (889 mg, 2.73 mmol, 3 eq) and Pd(dppf)Cl₂ (66.6 mg, 0.91 mmol, 0.1 eq) in dioxane (12 mL) and H₂O (1.2 mL) was degassed and purged with N₂ for 3 times, and then the mixture was stirred at 90° C. for 12 hours under N₂ atmosphere. The reaction mixture was partitioned between H₂O (10 mL) and EtOAc (10 mL). The organic phase was separated, washed with brine (20 mL), dried over anhydrous sodium sulfate, filtered and concentrated under reduced pressure to give a residue, which was purified by flash silica gel chromatography to give II-9-1 (140 mg, 0.271 mmol, 29.7% yield) as a yellow oil. LCMS: m/z 516.3 (M+1).

Step 7. To a solution of I1-9-1 (140 mg, 0.271 mmol, 1 eq) in CH₂Cl₂ (2 mL) was added TFA (1 mL). The mixture was stirred at 15° C. for 1 hour. The reaction mixture was concentrated under reduced pressure. The residue was diluted with NaHCO₃ (10 mL) and extracted with CH₂Cl₂ (5 mL×2). The combined organic layers were washed with brine (5 mL), dried over anhydrous sodium sulfate, filtered and concentrated under reduced pressure to give a residue, which was purified by flash silica gel chromatography to provide Ex. 6 (56.9 mg, 0.130 mmol, 48.0% yield) as an off-white solid.

Ex. 6-14 were prepared following General Method B.

### Preparation of triisopropyl-[2-[1-tetrahydropyran-2-yl-5-(4,4,5,5-tetramethyl-1,3,2-dioxaborolan-2-yl)indazol-3-yl]ethynyl]silane (III-1-1)

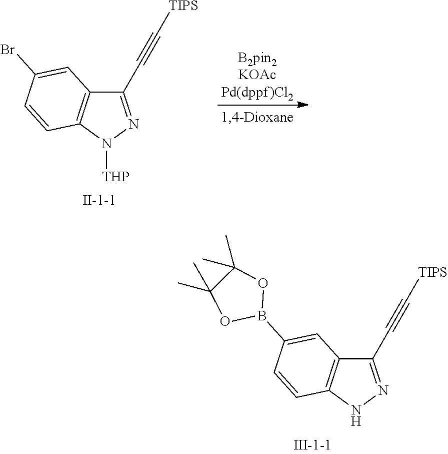

To 2-(5-bromo-1-tetrahydropyran-2-yl-indazol-3-yl)ethynyl-triisopropyl-silane (2 g, 4.33 mmol) in solvent, 1,4-Dioxane (20.86 mL), was added 4,4,5,5-tetramethyl-2-(4,4,5,5-tetramethyl-1,3,2-dioxaborolan-2-yl)-1,3,2-dioxaborolane (1.43 g, 5.63 mmol) and Potassium Acetate (1.28 g, 13.00 mmol). The mixture was stirred as argon was bubbled through for 5 minutes, followed by addition of catalyst, Pd(dppf)Cl₂ (158.55 mg, 216.68 μmol). The vessel sealed and heat to 85° C. for 18 hr. The reaction was diluted with DCM and water (80 mL) and the layers were separated. The aqueous layer was extracted again with DCM (2×40 mL). The combined organic layer was washed with brine and dried over sodium sulfate. Flash column chromatography provided triisopropyl-[2-[l-tetrahydropyran-2-yl-5-(4,4,5,5-tetramethyl-1,3,2-dioxaborolan-2-yl)indazol-3-yl]ethynyl]silane (1.87 g, 3.68 mmol, 84.85% yield). LCMS: [M+H]⁺m/z=509.28

### Preparation of tert-butyl N-[2-[(4-bromo-2-methyl-pyrazol-3-yl)methyl-methylamino]ethyl]-N-methyl-carbamate (III-2-1)

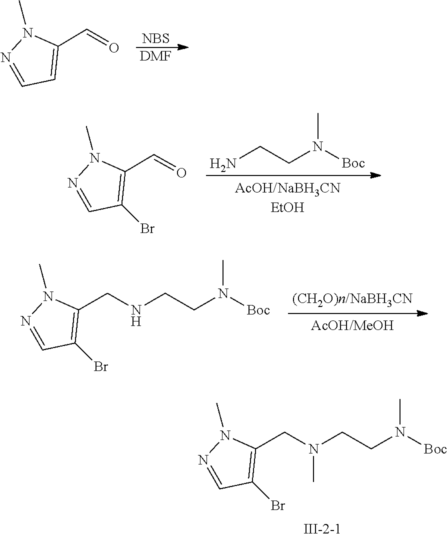

Step 1. To a solution of 2-methylpyrazole-3-carbaldehyde (1 g, 9.08 mmol, 1 eq) in DMF (15 mL) was added NBS (1.78 g, 9.99 mmol, 1.1 eq). The reaction was stirred at 25° C. for 12 hours. The residue was diluted with H₂O (50 mL) and extracted with EtOAc (50 mL*3). The combined organic layers were washed with brine (30 mL*2), dried over Na₂SO₄, filtered and concentrated under reduced pressure. The residue was purified by flash silica gel chromatography to give 4-bromo-2-methyl-pyrazole-3-carbaldehyde (1.3 g, 6.88 mmol, 75.7% yield) as white solid. ¹H NMR (400 MHz, DMSO-d₆) δ=9.84 (s, 1H), 7.77 (s, 1H), 4.08 (s, 3H).

Step 2. To a solution of 4-bromo-2-methyl-pyrazole-3-carbaldehyde (5 g, 26.45 mmol, 1 eq) in EtOH (100 mL) was added AcOH (159 mg, 2.65 mmol, 0.1 eq) and tert-butyl N-(2-aminoethyl)-N-methyl-carbamate (5.53 g, 31.7 mmol, 1.2 eq). The reaction was stirred at 80° C. for 12 hours. And then NaBH₃CN (4.99 g, 79.4 mmol, 3 eq) was added into the mixture and stirred at 25° C. for 1 hour. The reaction mixture was concentrated under reduced pressure and purified by reversed-phase HPLC to give tert-butyl N-[2-[(4-bromo-2-methyl-pyrazol-3-yl)methylamino]ethyl]-N-methyl-carbamate (5 g, 14.4 mmol, 54.4% yield) as yellow gum. ¹H NMR (400 MHz, DMSO-d₆) δ=7.43 (s, 1H), 3.84 (s, 3H), 3.75 (s, 2H), 3.19 (s, 2H), 2.75 (s, 3H), 2.57 (t, J=6.4 Hz, 2H), 1.43-1.30 (m, 9H).

Step 3. To a solution of tert-butyl N-[2-[(4-bromo-2-methyl-pyrazol-3-yl)methylamino]ethyl]-N-methyl-carbamate (5 g, 14.4 mmol, 1 eq) in MeOH (50 mL) was added HCHO (2.34 g, 28.8 mmol, 37% purity, 2 eq), AcOH (86.5 mg, 1.44 mmol, 0.1 eq) and NaBH₃CN (2.71 g, 43.2 mmol, 3 eq). The reaction was stirred at 25° C. for 2 hours. The reaction mixture was concentrated under reduced pressure. The crude product was purified by reversed-phase HPLC to give tert-butyl N-[2-[(4-bromo-2-methyl-pyrazol-3-yl)methyl-methylamino]ethyl]-N-methyl-carbamate (3.2 g, 8.86 mmol, 61.5% yield) as a yellow gum. ¹H NMR (400 MHz, DMSO-d₆) δ=7.46 (s, 1H), 3.80 (s, 3H), 3.52 (s, 2H), 3.29-3.13 (m, 2H), 2.76-2.55 (m, 3H), 2.40 (d, J=5.6 Hz, 2H), 2.29-2.07 (m, 3H), 1.42-1.26 (m, 9H).

### General Method C: Preparation of (18E)-8,10,13,15,17-pentamethyl-2,8,9,10,11,12,13,15-octahydro-14H-3,5-ethenotripyrazolo[3,4-f:3′,4′-j:4″,3″-n][1,4]diazacyclohexadecin-14-one (Ex. 15)

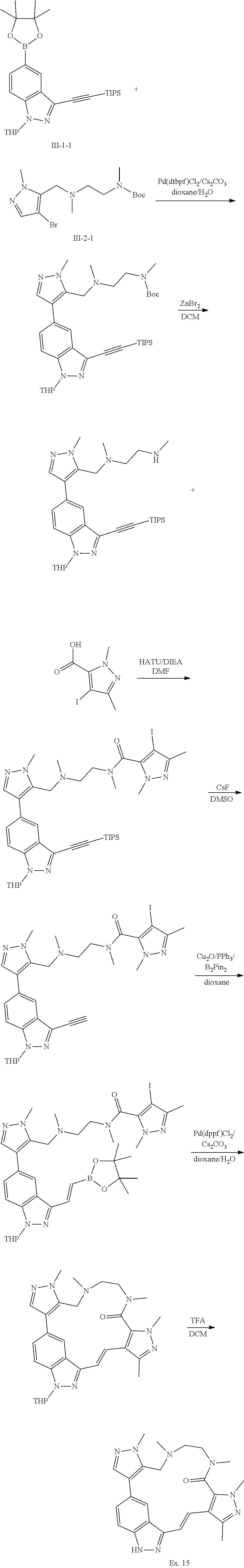

Step 1. A mixture of III-1-1 (700 mg, 1.38 mmol, 1.1 eq), III-2-1 (452 mg, 1.25 mmol, 1 eq), ditert-butyl(cyclopentyl)phosphane;dichloropalladium;iron (81.6 mg, 0.125 mmol, 0.1 eq), Cs₂CO₃ (1.22 g, 3.75 mmol, 3 eq) in dioxane (10 mL) and H₂O (2 mL) was degassed and purged with N₂ for 3 times, and then the mixture was stirred at 80° C. for 2 hours under N₂ atmosphere. The reaction mixture was concentrated under reduced pressure to remove solvent. The residue was purified by flash silica gel chromatography to give tert-butyl N-methyl-N-[2-[methyl-[[2-methyl-4-[1-tetrahydropyran-2-yl-3-(2-triisopropylsilylethynyl)indazol-5-yl]pyrazol-3-yl]methyl]amino]ethyl]carbamate (182 mg, 0.239 mmol, 19.1% yield) as a yellow solid. LCMS: m/z 663.6 (M+1)

tert-Butyl N-methyl-N-[2-[methyl-[[2-methyl-4-[1-tetrahydropyran-2-yl-3-(2-triisopropylsilylethynyl)indazol-5-yl]pyrazol-3-yl]methyl]amino]ethyl]carbamate was converted to Ex. 15 using similar methods as Step 2-Step 6 in General Method B.

### General Method D: Preparation of (17E)-14-(2-hydroxyethyl)-8,12,16-trimethyl-2,11,12,14-tetrahydro-8H-3,5-ethenotripyrazolo[3,4-f:3′,4′-j:4″,3″-n][1,4]oxazacyclopentadecin-13(10H)-one (Ex. 16)

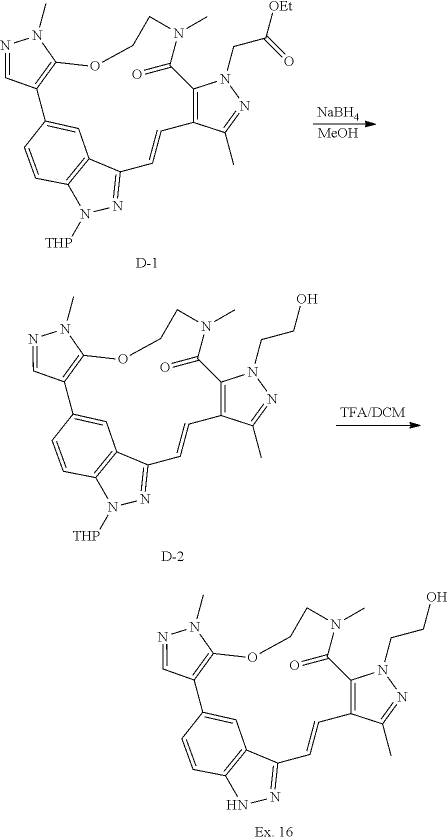

Step 1. D-1 was prepared following General Method B. To a solution of D-1 (30.8 mg, 0.536 mmol, 1 eq) in MeOH (2 mL) was added NaBH₄ (40.5 mg, 1.07 mmol, 20 eq) at 0° C. The mixture was stirred at 0° C. for 3 hr. On completion, the mixture was quenched with water (20 mL) and extracted with ethyl acetate (25 mL×3). The combined organic phase was dried over anhydrous sodium sulfate, filtered and concentrated to give D-2 (400 mg, crude) as a white solid. LCMS: m/z 532.2 (M+1).

Step 2. To a solution of D-2 (28.0 mg, 0.526 mmol, 1 eq) in DCM (1 mL) was added TFA (1.54 g, 13.5 mmol, 256 eq). The mixture was stirred at 25° C. for 1 hr. On completion, the mixture was concentrated. The crude product was purified by prep HPLC to give Ex. 16 (4.49 mg, 0.100 mmol, 19% yield) as a white solid.

Ex. 17 was prepared following General Method D.

### General Method E: Preparation of (17E)-8,12,16-trimethyl-14-[2-(pyrrolidin-1-yl)ethyl]-2,11,12,14-tetrahydro-8H-3,5-ethenotripyrazolo[3,4-f:3′,4′-j:4″,3″-n][1,4]oxazacyclopentadecin-13(10H)-one (Ex. 18)

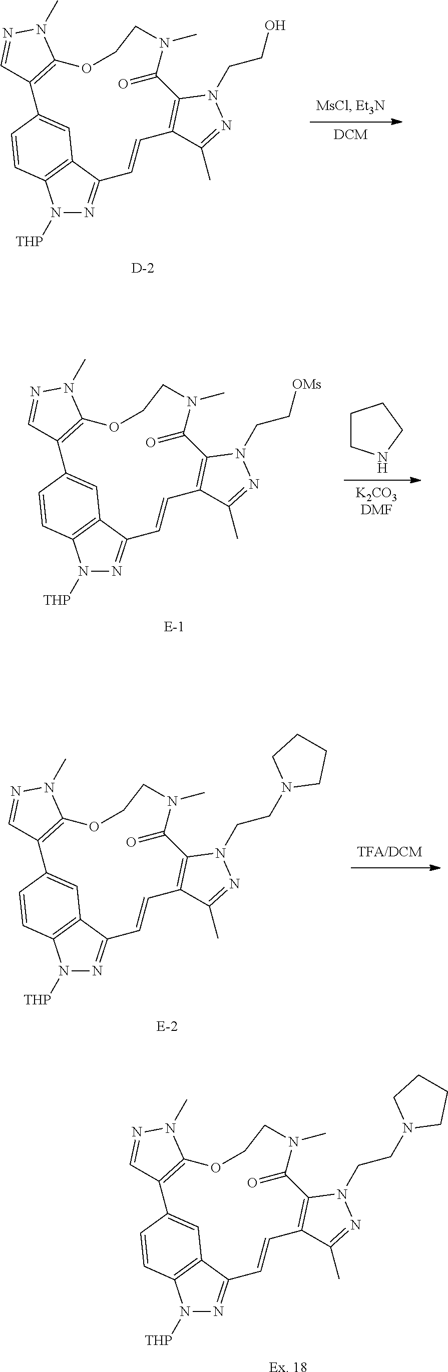

Step 1. To a solution of D-2 (50.0 mg, 0.094 mmol, 1 eq) in DCM (2 mL) was added Et₃N (47.5 mg, 0.470 mmol, 5 eq) and then MsCl (64.6 mg, 0.564 mmol, 6 eq) was added dropwise at 0° C. The mixture was stirred at 0° C. for 2 hr. On completion, the mixture was quenched with water (20 mL) and extracted with DCM (25 mL×3). The combined organic phase was dried over anhydrous sodium sulfate, filtered and concentrated to give E-1 (57.0 mg, 0.0934 mmol, 99% yield) as a yellow oil.

Step 2. To a solution of E-1 (57.0 mg, 0.0934 mmol, 1 eq) and pyrrolidine (9.97 mg, 0.140 mmol, 1.5 eq) in DMF (2 mL) was added K₂CO₃ (38.7 mg, 0.280 mmol, 3 eq). The mixture was stirred at 80° C. for 12 hr. On completion, the mixture was quenched with water (20 mL) and extracted with ethyl acetate (25 mL×3). The combined organic phase was dried over anhydrous sodium sulfate, filtered and concentrated to give E-2 (54.0 mg, 0.0923 mmol, 98% yield) as yellow oil. LCMS: m/z 585.3 (M+1).

Step 3. To a solution of E-2 (50.0 mg, 0.0855 mmol, 1 eq) in DCM (1 mL) was added TFA (1.54 g, 13.5 mmol, 157 eq). The mixture was stirred at 25° C. for 2 hr. On completion, the mixture was filtered and concentrated. The crude product was purified by prep HPLC to give Ex. 18 (4.54 mg, 0.009 mmol, 10% yield) as a yellow solid.

Ex. 19 was prepared following General Method E.

### Preparation of 5-ethoxy-4-iodo-2-methyl-pyrazole-3-carboxylic acid (I-22)

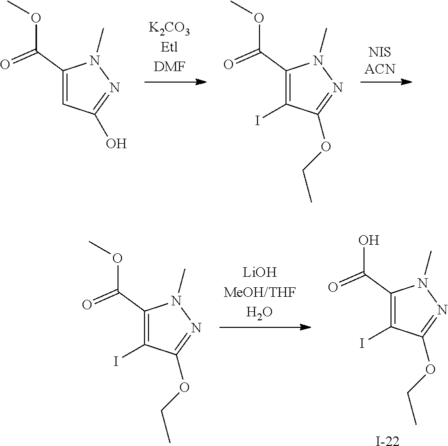

Step 1. To a solution of methyl 5-hydroxy-2-methyl-pyrazole-3-carboxylate (800 mg, 5.12 mmol, 1 eq) in DMF (10 mL) was added K₂CO₃ (2.12 g, 15.4 mmol, 3 eq) and EtI (799 mg, 5.12 mmol, 1 eq), then the mixture was stirred at 80° C. for 2 h. On completion, the reaction mixture was diluted with H₂O (30 mL) and extracted with EA (10 mL×3). The combined organic layers were washed with Brine (10 mL×2), dried over Na₂SO₄, filtered and concentrated under reduced pressure to give a residue. The residue was purified by flash silica gel chromatography (ISCO®; 12 g SepaFlash® Silica Flash Column, Eluent of 0-30% THF/Petroleum ether gradient@30 mL/min) to give methyl 5-ethoxy-2-methyl-pyrazole-3-carboxylate (780 mg, 4.23 mmol, 82.65% yield) as a yellow oil.

Step 2. To a solution of methyl 5-ethoxy-2-methyl-pyrazole-3-carboxylate (750 mg, 4.07 mmol, 1 eq) in ACN (10 mL) was added NIS (1.83 g, 8.14 mmol, 2 eq) at 0° C., then the mixture was stirred at 60° C. for 12 h. On completion, the reaction mixture was quenched by addition saturated Na₂SO₃ (20 mL) at 25° C., and then diluted with H₂O (10 mL) and extracted with EA (10 mL×3). The combined organic layers were washed with Brine (10 mL×2), dried over Na₂SO₄, filtered and concentrated under reduced pressure to give methyl 5-ethoxy-4-iodo-2-methyl-pyrazole-3-carboxylate (1.53 g, crude) as a yellow solid.

Step 3. To a solution of methyl 5-ethoxy-4-iodo-2-methyl-pyrazole-3-carboxylate (1.5 g, 4.84 mmol, 1 eq) in MeOH (3 mL), THE (6 mL) and H₂O (3 mL) was added LiOH·H₂O (609 mg, 14.5 mmol, 3 eq), then the mixture was stirred at 25° C. for 2 h. On completion, the pH was adjusted to 4 with 1 M HCl. The precipitate was collected by filtration. The precipitate was triturated in H₂O and collected by filtration to give 5-ethoxy-4-iodo-2-methyl-pyrazole-3-carboxylic acid (1.00 g, 3.38 mmol, 70% yield) as a yellow solid.

Ex. 22 was prepared following General Method B using the I-22 in step 3.

### Preparation of tert-butyl N-[(2S)-2-[4-(3-ethynyl-1-tetrahydropyran-2-yl-indazol-5-yl)-2-methyl-pyrazol-3-yl]oxypropyl]-N-methyl-carbamate (I-23)

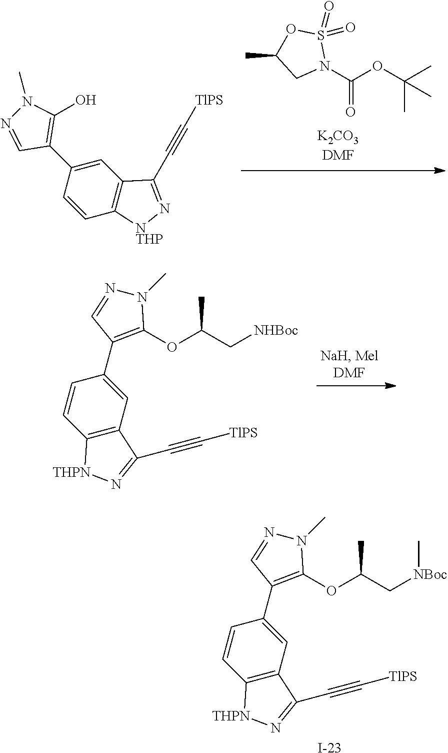

Step 1. To 2-methyl-4-[1-tetrahydropyran-2-yl-3-(2-triisopropylsilylethynyl)indazol-5-yl]pyrazol-3-ol (3 g, 6.27 mmol) in DMF (31.33 mL) was added potassium carbonate (2.60 g, 18.80 mmol) followed by tert-butyl (5R)-5-methyl-2,2-dioxo-oxathiazolidine-3-carboxylate (1.78 g, 7.52 mmol). The mixture was stirred at 80° C. for 18 h, diluted with DCM (50 mL) and cooled. The mixture was then filtered through a celite pad and the filtrate was concentrated. The remaining residue was worked up with DCM and water (50 mL) and the layers were separated. The aqueous layer was extracted again with DCM (2×25 mL). The combined organic layer was washed with brine and dried over sodium sulfate. Flash column chromatography (automated system, 40 g silica, 0-70% EA in Hexanes) provided tert-butyl N-[(2S)-2-[2-methyl-4-[I1-tetrahydropyran-2-yl-3-(2-triisopropylsilylethynyl)indazol-5-yl]pyrazol-3-yl]oxypropyl]carbamate (2.72 g, 4.28 mmol, 68.25% yield).

Step 2. To tert-butyl N-[(2S)-2-[2-methyl-4-[1-tetrahydropyran-2-yl-3-(2-triisopropylsilylethynyl)indazol-5-yl]pyrazol-3-yl]oxypropyl]carbamate (2.2 g, 3.46 mmol) in anhydrous DMF (17 mL) was added Sodium hydride (145.29 mg, 3.63 mmol, 60% purity) at 0° C. The mixture was stirred for 1 h and Methyl Iodide (540.2 mg, 3.81 mmol, 237 μL) was added. The reaction was stirred as temperature increased to ambient, and the mixture was allowed to stir overnight. The reaction was then quenched with saturated ammonium chloride (aq) carefully (˜1 mL) at 0° C. The mixture was worked up with DCM and water (10 mL). The aqueous layer was extracted with DCM (2×5 mL) and the combined organic layer was washed with brine and dried over sodium sulfate. Flash column chromatography (automated system, 12 g silica, 0-100% EA in Hexanes) provided tert-butyl N-[(2S)-2-[4-(3-ethynyl-1-tetrahydropyran-2-yl-indazol-5-yl)-2-methyl-pyrazol-3-yl]oxypropyl]-N-methyl-carbamate (456 mg, 0.924 mmol, 26.70% yield).

Ex. 23 was prepared following General Method B using I-23 in Step 2.

### Preparation of 5-bromo-4-iodo-2-methyl-pyrazole-3-carboxylic acid (I-24)

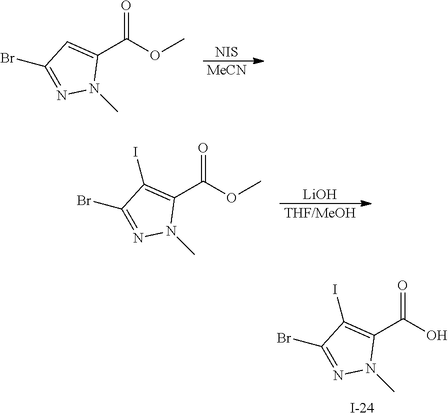

Step 1. To methyl 5-bromo-2-methyl-pyrazole-3-carboxylate (200 mg, 0.913 mmol) in acetonitrile (4 mL) was added NIS (616 mg, 2.7 mmol), and the mixture was stirred at 85° C. for 18 h. The reaction was then quenched with water (4 mL), diluted with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-40% EA in Hexanes) provided methyl 5-bromo-4-iodo-2-methyl-pyrazole-3-carboxylate (231 mg, 0.670 mmol, 73.34% yield).

Step 2. To methyl 5-bromo-4-iodo-2-methyl-pyrazole-3-carboxylate (231 mg, 0.670 mmol) in THF (2 mL) was added LiOH (2 M, 0.4 mL, aq), and the mixture was stirred at 22° C. for 4 h. The reaction was then cooled in −20° C. freezer, diluted with DCM (5 mL), and 2M HCl (aq., 0.5 mL) was added with vigorous stirring. The reaction was then diluted with DCM and water (20 mL) and the layers were separated. The aqueous layer was extracted again with DCM (2×10 mL). The combined organic layer was washed with brine and dried over sodium sulfate. The solids were filtered and washed with DCM to afford 5-bromo-4-iodo-2-methyl-pyrazole-3-carboxylic acid (223 mg, 0.674 mmol, 100% yield).

### General Method F: Preparation of (17E)-8,12,14-trimethyl-13-oxo-2,10,11,12,13,14-hexahydro-8H-3,5-ethenotripyrazolo[3,4-f:3′,4′-j:4″,3″-n][1,4]oxazacyclopentadecine-16-carbonitrile (Ex. 24)

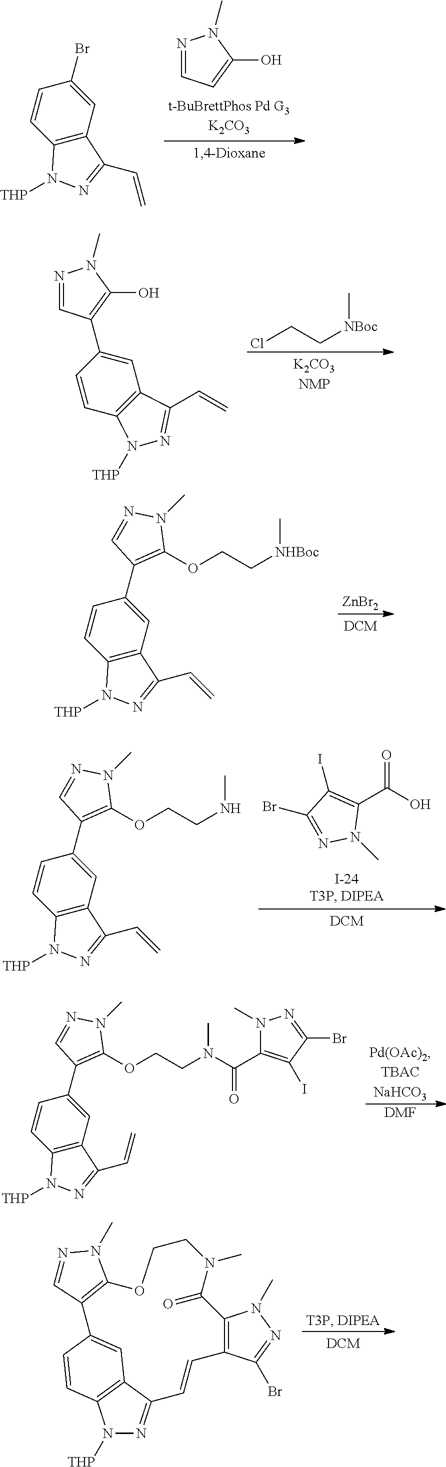

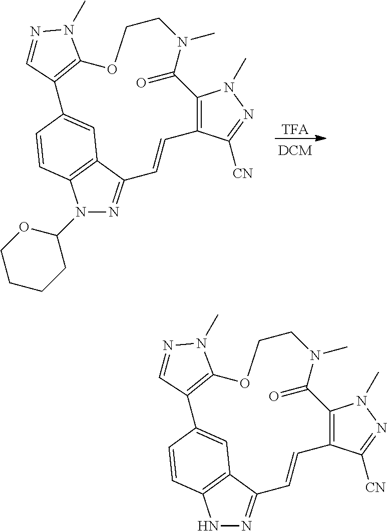 [SMILES: CN1CCOc2c(cnn2C)-c2ccc3c(c2)c(nn3C2CCCCO2)/C=C/c2c(C#N)nn(C)c2C1=O ; CN1CCOc2c(cnn2C)-c2ccc3[nH]nc(c3c2)/C=C/c2c(C#N)nn(C)c2C1=O ; "TFA"]

Step 1. 5-bromo-1-tetrahydropyran-2-yl-3-vinyl-indazole (10.08 g, 32.81 mmol), 2-methylpyrazol-3-ol (4.18 g, 42.66 mmol) and Potassium Carbonate, anhydrous powder 325 mesh (11.34 g, 82.04 mmol) were added into a vial with dioxane (82.04 mL). Argon stream was bubbled for 15 minutes. i-BuBrettPhos Pd G3 (1.12 g, 1.31 mmol) was added under Argon. The vial was sealed and stirred at 100° C. Water (800 mL) was added to the reaction mixture, followed by 20% K₂CO₃ aq. solution (150 mL) to adjust the pH to 9-10, followed by extraction with ether (100 mL×2). The combined ether layers were washed with 20 ml 20% K₂CO₃ aq. solution. The aqueous layer was cooled in an ice bath and acidified with NaHSO₄ to pH-6 and was extracted with 10% MeOH/DCM (200 mL×6). The combined organic layers were washed with water and brine, dried over Na₂SO₄. The solvents were removed in vacuo and the residue was dissolved in DCM (50 mL), added 15 w/w % activated charcoal (0.825 g), 20 w/w % trisamine resin (1.1 g), and stirred for 3 hours. The suspension was then filtered through a celite cake, and the cake was washed with DCM (20 mL). The filtrate was evaporated to furnish 2-methyl-4-(1-tetrahydropyran-2-yl-3-vinyl-indazol-5-yl)pyrazol-3-ol, which was carried over to the next step without further purification.

Step 2. To 2-methyl-4-(1-tetrahydropyran-2-yl-3-vinyl-indazol-5-yl)pyrazol-3-ol (250 mg, 0.771 mmol) in NMP (3.85 mL) was added base, potassium carbonate (320 mg, 2.3 mmol) followed by tert-butyl N-(2-chloroethyl)-N-methyl-carbamate (224 mg, 1.2 mmol). Stirred at 90° C. for 2 hr. Reaction was cooled and diluted with DCM (20 mL). Filtered through a microfilter and filtrate was worked up with DCM and water (25 mL) and the layers were separated. The aqueous layer was extracted again with DCM (2×10 mL). The combined organic layer was washed with brine and dried over sodium sulfate. Flash column chromatography twice (automated system, 12 g silica, 0-50% EA in Hexanes) provided tert-butyl N-methyl-N-[2-[2-methyl-4-(1-tetrahydropyran-2-yl-3-vinyl-indazol-5-yl)pyrazol-3-yl]oxyethyl]carbamate (198 mg, 0.411 mmol, 53.35% yield).

Step 3. To tert-butyl N-methyl-N-[2-[2-methyl-4-(1-tetrahydropyran-2-yl-3-vinyl-indazol-5-yl)pyrazol-3-yl]oxyethyl]carbamate (198 mg, 0.411 mmol) in DCM (2 mL) was added zinc bromide (370 mg, 1.6 mmol), and the reaction was stirred at 22° C. for 2 days. The reaction was diluted with DCM and water (20 mL) and the layers were separated. The aqueous layer was extracted again with DCM (2×10 mL). The combined organic layer was washed with brine and dried over sodium sulfate. Flash column chromatography (automated system, 12 g silica, 0-20% Methanol in DCM) provided N-methyl-2-[2-methyl-4-(1-tetrahydropyran-2-yl-3-vinyl-indazol-5-yl)pyrazol-3-yl]oxy-ethanamine (63 mg, 0.165 mmol, 40.17% yield).

Step 4. To 5-bromo-4-iodo-2-methyl-pyrazole-3-carboxylic acid (66 mg, 198.2 μmol) in DCM (1 mL) was added N-methyl-2-[2-methyl-4-(1-tetrahydropyran-2-yl-3-vinyl-indazol-5-yl)pyrazol-3-yl]oxy-ethanamine (63 mg, 0.165 mmol) and DIPEA (1.65 mmol, 288 μL), followed by T3P (0.330 mmol, 193 μL, 50% in EA). The mixture was stirred at 40° C. for 18 h. The reaction was then diluted with DCM and water (5 mL) and the layers were separated. The aqueous layer was extracted again with DCM (2×3 mL). The combined organic layer was washed with brine and dried over sodium sulfate. Flash column chromatography (automated system, 12 g silica, 40-100% EA in Hexanes) provided 5-bromo-4-iodo-N,2-dimethyl-N-[2-[2-methyl-4-(1-tetrahydropyran-2-yl-3-vinyl-indazol-5-yl)pyrazol-3-yl]oxyethyl]pyrazole-3-carboxamide (75 mg, 0.108 mmol, 65.40% yield).

Step 5. To 5-bromo-4-iodo-N,2-dimethyl-N-[2-[2-methyl-4-(1-tetrahydropyran-2-yl-3-vinyl-indazol-5-yl)pyrazol-3-yl]oxyethyl]pyrazole-3-carboxamide (75 mg, 0.108 mmol) in anhydrous DMF (2 mL) was added sodium bicarbonate (28 mg, 0.324 mmol), and TBAC (33 mg, 0.119 mmol). The mixture was stirred while Argon was bubbled through and catalyst, palladium acetate (3 mg, 0.011 mmol) was added. Argon was bubbled through for an additional 5 minutes. The vessel was then sealed, and the reaction was heated to 140° C. for 1.5 h. The reaction was diluted with DCM and water (20 mL) and the layers were separated. The aqueous layer was extracted again with DCM (2×10 mL). The combined organic layer was washed with brine and dried over sodium sulfate. Flash column chromatography (automated system, 12 g silica, 0-10% Methanol in DCM) provided (17E)-16-bromo-8,12,14-trimethyl-2-(oxan-2-yl)-2,11,12,14-tetrahydro-8H-3,5-ethenotripyrazolo[3,4-f:3′,4′-j:4″,3″-n][1,4]oxazacyclopentadecin-13(10H)-one (58 mg, 0.102 mmol, 94.80% yield).

Step 6. To (13E)-22-bromo-29,30,32-trimethyl-31-tetrahydropyran-2-yl-34-oxa-26,27,28,29,30,31,32-heptazapentacyclohexacosa-3,5(15),6(26),13,16,18(23),19(21),20(27),22(28)-nonaen-25-one (58 mg, 0.102 mmol) in DMA (1 mL) was added Zinc (13.5 mg, 0.205 mmol) and zinc cyanide (132 mg, 1.13 mmol), argon was bubbled through as dppf (34 mg, 0.061 mmol) was added followed by Pd(dba)₂ (18 mg, 0.030 mmol). The mixture was stirred under argon for about 5 minutes. The vessel was closed and heated to 120° C. and stirred for 18 h. The reaction was diluted with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-10% Methanol in DCM) provided (17E)-8,12,14-trimethyl-2-(oxan-2-yl)-13-oxo-2,10,11,12,13,14-hexahydro-8H-3,5-ethenotripyrazolo[3,4-f:3′,4′-j:4″,3″-n][1,4]oxazacyclopentadecine-16-carbonitrile (30 mg, 0.585 mmol, 57.16% yield).

Step 7. To (13E)-31,32,34-trimethyl-26-oxo-33-tetrahydropyran-2-yl-36-oxa-28,29,30,31,32,33,34-heptazapentacyclohexacosa-3,5(16),6(28),13,17,19(24),20(23),21(29),22(30)-nonaene-22-carbonitrile (30 mg, 0.585 mmol) in DCM (1 mL) was added TFA (6.53 mmol, 0.5 mL). The mixture was stirred at 22° C. for 2 h. The volatiles were removed under reduced pressure, and 0.5 mL of Triethylamine added. Flash column chromatography (automated system, 12 g silica, 0-10% MeOH in DCM) followed by trituration in DCM/Ethanol (0.2/2 mL) provided (17E)-8,12,14-trimethyl-13-oxo-2,10,11,12,13,14-hexahydro-8H-3,5-ethenotripyrazolo[3,4-f:3′,4′-j:4″,3″-n∥1,4|oxazacyclopentadecine-16-carbonitrile (15 mg, 0.035 mmol, 59.33% yield, 99.18% purity) after filtration (Ex. 24).

### Preparation of 3-ethyl-4-iodo-isoxazole-5-carboxylic acid

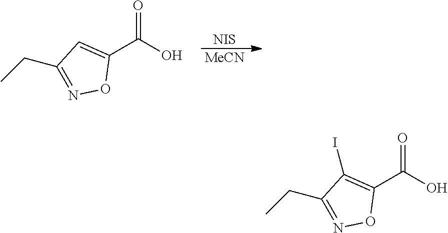

To 3-ethylisoxazole-5-carboxylic acid (296 mg, 2.10 mmol) in TFA (5 mL) was added NIS (566 mg, 2.5 mmol). The mixture was stirred at 70° C. for 30 min. The reaction was diluted with DCM and water (25 mL) and the layers were separated. The aqueous layer was extracted again with DCM (2×15 mL). The combined organic layer was washed with brine and dried over sodium sulfate. Flash column chromatography (automated system, 12 g silica, 20-60% EA in Hexanes) provided 3-ethyl-4-iodo-isoxazole-5-carboxylic acid (109 mg, 0.408 mmol, 19.46% yield).

Ex. 25 was prepared by General Method F using the above 3-ethyl-4-iodo-isoxazole-5-carboxylic acid in Step 1, following Steps 1-5 and Step 7.

### Preparation of 2-[2-[tert-butyl(dimethyl)silyl]oxyethyl]-4-iodo-5-methyl-pyrazole-3-carboxylic acid

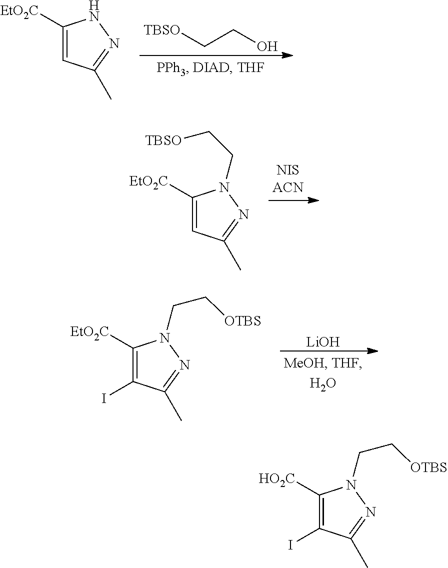

Step 1: To a solution of methyl 3-methyl-1H-pyrazole-5-carboxylate (1.00 g, 7.14 mmol, 1 eq) and 2-[tert-butyl(dimethyl)silyl]oxyethanol (2.52 g, 14.3 mmol, 2 eq) in THF (10 mL) was added PPh₃ (4.12 g, 15.7 mmol, 2.2 eq), the mixture was degassed and purged with N₂ for 3 times and stirred at 25° C. for 30 mins, and then DIAD (3.17 g, 15.7 mmol, 2.2 eq) was added dropwise at 0° C. The mixture was stirred at 25° C. for 12 h under N₂ atmosphere. On completion, the mixture was concentrated to give a residue. The residue was purified by column chromatography (SiO₂, PE/THF=1:0 to 8:1) to give methyl 2-[2-[tert-butyl(dimethyl)silyl]oxyethyl]-5-methyl-pyrazole-3-carboxylate (1.84 g, 6.16 mmol, 86% yield) as a colorless oil.

Step 2: To a solution of methyl 2-[2-[tert-butyl(dimethyl)silyl]oxyethyl]-5-methyl-pyrazole-3-carboxylate (1.63 g, 5.46 mmol, 1 eq) in Acetonitrile (17 mL) was added NIS (3.69 g, 16.38 mmol, 3 eq) at 0° C. The mixture was stirred at 80° C. for 12 h. On completion, the mixture was quenched with sat. Na₂SO₃ (25 mL) and extracted with ethyl acetate (25 mL×3), the combined organic phase was dried over anhydrous sodium sulfate, filtered and concentrated to give a residue. The residue was purified by column chromatography (SiO₂, PE/THF=1:0 to 15:1) to give methyl 2-[2-[tert-butyl(dimethyl)silyl]oxyethyl]-4-iodo-5-methyl-pyrazole-3-carboxylate (2.13 g, 5.02 mmol, 92% yield) as a yellow oil.

Step 3: To a solution of methyl 2-[2-[tert-butyl(dimethyl)silyl]oxyethyl]-4-iodo-5-methyl-pyrazole-3-carboxylate (1.00 g, 2.36 mmol, 1 eq) in MeOH (2 mL), THF (4 ml) and H₂O (2 mL) was added LiOH·H₂O (98.9 mg, 2.36 mmol, 1 eq). The mixture was stirred at 0° C. for 4 h. On completion, the mixture was quenched with 1M HCl (5 mL) and extracted with 2-MeTHF (15 mL×3), the combined organic phase was dried over anhydrous sodium sulfate, filtered and concentrated to give 2-[2-[tert-butyl(dimethyl)silyl]oxyethyl]-4-iodo-5-methyl-pyrazole-3-carboxylic acid (783 mg, 1.91 mmol, 81% yield) as a white solid.

To a solution of tert-butyl N-methyl-N-[2-[methyl-[[2-methyl-4-(1-tetrahydropyran-2-yl-3-vinyl-indazol-5-yl)pyrazol-3-yl]methyl]amino]ethyl]carbamate (700 mg, 1.38 mmol, 1 eq) in Acetonitrile (7 mL) was added TMSI (358 mg, 1.79 mmol, 1.3 eq) at 0° C. The mixture was stirred at 0° C. for 2 h. On completion, the mixture was quenched with sat. NaHCO₃ (20 mL) and extracted with ethyl acetate (25 mL×3), the combined organic phase was dried over anhydrous sodium sulfate, filtered and concentrated to give a residue. The residue was purified by column chromatography (SiO₂, DCM/MeOH=1:0 to 5:1) to give N, N-dimethyl-N-[[2-methyl-4-(1-tetrahydropyran-2-yl-3-vinyl-indazol-5-yl) pyrazol-3-yl]methyl]ethane-1,2-diamine (100 mg, 0.245 mmol, 18% yield) as a yellow oil.

### Preparation of (2-[2-[tert-butyl(dimethyl)silyl]oxyethyl]-4-iodo-N,5-dimethyl-N-[2-[methyl-[[2-methyl-4-(1-tetrahydropyran-2-yl-3-vinyl-indazol-5-yl)pyrazol-3-yl]methyl]amino]ethyl]pyrazole-3-carboxamide

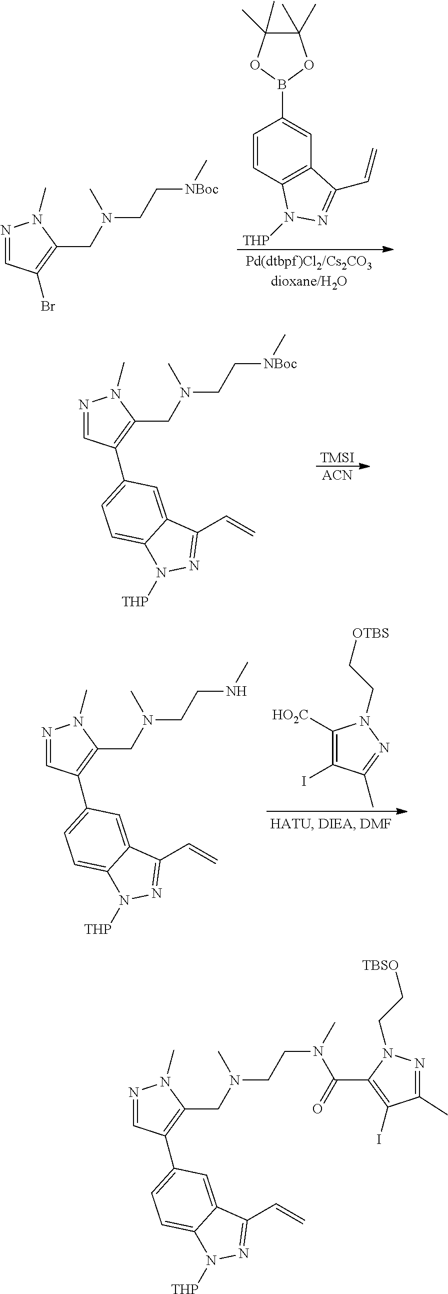

Step 1: A mixture of tert-butyl N-[2-[(4-bromo-2-methyl-pyrazol-3-yl)methyl-methylamino]ethyl]-N-methyl-carbamate (Intermediate III-2-1, 1.22 g, 3.39 mmol, 2 eq), 1-tetrahydropyran-2-yl-5-(4,4,5,5-tetramethyl-1,3,2-dioxaborolan-2-yl)-3-vinyl-indazole (600 mg, 1.69 mmol, 1 eq, prepared using General Method F, Step 1), K₂CO₃ (702 mg, 5.08 mmol, 3 eq), Pd(dppf)Cl₂·CH₂Cl₂ (138 mg, 0.169 mmol, 0.1 eq) in dioxane (12 mL) and H₂O (1.5 mL) was degassed and purged with N₂ for 3 times, and then the mixture was stirred at 100° C. for 12 h under N₂ atmosphere. On completion, the mixture was filtered and concentrated to give a residue. The residue was purified by column chromatography (SiO₂, PE/THF=1:0 to 2:1) to give tert-butyl N-methyl-N-[2-[methyl-[[2-methyl-4-(1-tetrahydropyran-2-yl-3-vinyl-indazol-5-yl) pyrazol-3-yl]methyl]amino]ethyl]carbamate (699 mg, 1.37 mmol, 81% yield) as a yellow oil.

Step 2: To a solution of tert-butyl N-methyl-N-[2-[methyl-[[2-methyl-4-(1-tetrahydropyran-2-yl-3-vinyl-indazol-5-yl)pyrazol-3-yl]methyl]amino]ethyl]carbamate (700 mg, 1.38 mmol, 1 eq) in Acetonitrile (7 mL) was added TMSI (358 mg, 1.79 mmol, 1.3 eq) at 0° C. The mixture was stirred at 0° C. for 2 h. On completion, the mixture was quenched with sat. NaHCO₃ (20 mL) and extracted with ethyl acetate (25 mL×3), the combined organic phase was dried over anhydrous sodium sulfate, filtered and concentrated to give a residue. The residue was purified by column chromatography (SiO₂, DCM/MeOH=1:0 to 5:1) to give N, N-dimethyl-N-[[2-methyl-4-(1-tetrahydropyran-2-yl-3-vinyl-indazol-5-yl) pyrazol-3-yl]methyl]ethane-1,2-diamine (100 mg, 0.245 mmol, 18% yield) as a yellow oil.

Step 3: To a solution of 2-[2-[tert-butyl(dimethyl)silyl]oxyethyl]-4-iodo-5-methyl-pyrazole-3-carboxylic acid (80.4 mg, 0.196 mmol, 1 eq) in DMF (1 mL) was added HATU (89.4 mg, 0.235 mmol, 1.2 eq), DIEA (75.9 mg, 0.587 mmol, 3 eq) and stirred at 25° C. for 30 min, then N,N-dimethyl-N-[[2-methyl-4-(1-tetrahydropyran-2-yl-3-vinyl-indazol-5-yl)pyrazol-3-yl]methyl]ethane-1,2-diamine (80.0 mg, 196 mmol, 1 eq) was added into the mixture and stirred at 25° C. for 1 h. On completion, the mixture was quenched with water (5 mL) and extracted with ethyl acetate (5 mL×3), the combined organic phase was dried over anhydrous sodium sulfate, filtered and concentrated to give a residue. The residue was purified by column chromatography (SiO₂, PE/THF=1:0 to 2:1) to give 2-[2-[tert-butyl(dimethyl)silyl]oxyethyl]-4-iodo-N,5-dimethyl-N-[2-[methyl-[[2-methyl-4-(1-tetrahydropyran-2-yl-3-vinyl-indazol-5-yl)pyrazol-3-yl]methyl]amino]ethyl]pyrazole-3-carboxamide (69.0 mg, 0.086 mmol, 44% yield) as a yellow oil.

Ex. 26 was prepared following General method F from the above intermediate following Steps 5 and 7.

### Preparation of 2-[2-[tert-butyl(dimethyl)silyl]oxyethyl]-5-ethoxy-4-iodo-pyrazole-3-carboxylic acid

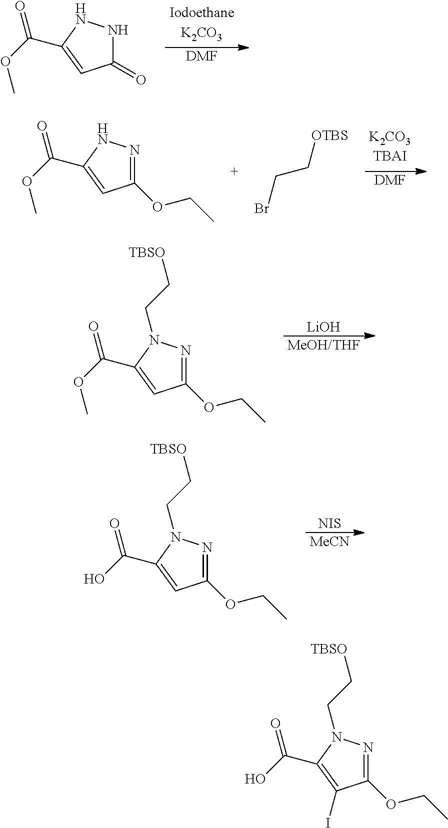

Step 1. To ethyl 3-hydroxy-1H-pyrazole-5-carboxylate (250 mg, 1.60 mmol) in DMF (8 mL) was added potassium carbonate (664 mg, 4.8 mmol) followed by ethyl iodide (1.60 mmol, 130 μL). The mixture was stirred at 80° C. for 2 h. The reaction was cooled and diluted with DCM (20 mL), then filtered. The filtrate was worked up with DCM and water (100 mL). After separation of layers, the aqueous layer was extracted with DCM again (2×50 mL). The combined organic layers were washed with brine and then dried over sodium sulfate. Flash column chromatography (automated system, 12 g silica, 0-30% EA in Hexanes) provided ethyl 3-ethoxy-1H-pyrazole-5-carboxylate (184 mg, 0.999 mmol, 62.39% yield).

Step 2. To ethyl 3-ethoxy-1H-pyrazole-5-carboxylate (184 mg, 998.96 μmol) in DMF (5 mL) was added potassium carbonate (414 mg, 3 mmol), followed by 2-bromoethoxy-tert-butyl-dimethyl-silane (1.50 mmol, 322 μL) and TBAI (369 mg, 999 μmol). The mixture was stirred at 60° C. for 6 hr. The reaction was diluted with DCM and cooled in an ice bath and the mixture was then filtered and washed with more DCM. The filtrate was concentrated to dryness and the residue was purified by Flash column chromatography (automated system with ELSD, 12 g silica, 0-20% EA in Hexanes) provided ethyl 2-[2-[tert-butyl(dimethyl)silyl]oxyethyl]-5-ethoxy-pyrazole-3-carboxylate (220 mg, 0.642 mmol, 64.30% yield).

Step 3. To ethyl 2-[2-[tert-butyl(dimethyl)silyl]oxyethyl]-5-ethoxy-pyrazole-3-carboxylate (150 mg, 0.438 mmol) in Methanol (0.2 mL) and THE (1 ml) was added LiOH (2 M, 1 mL) in water. The mixture was stirred at 22° C. for 18 h. The reaction was cooled in −20° C. freezer and diluted with DCM and 2M HCl (aq) (1 mL) was added with vigorous stirring. The reaction was then diluted with DCM and water (5 mL), and the layers were separated. The aqueous layer was extracted again with DCM (2×5 mL). The combined organic layer was washed with brine and dried over sodium sulfate. Flash column chromatography (automated system, 12 g silica, 0-12.5% MeOH in DCM) provided 2-[2-[tert-butyl(dimethyl)silyl]oxyethyl]-5-ethoxy-pyrazole-3-carboxylic acid (36 mg, 114.48 mol, 26.14% yield) and 5-ethoxy-2-(2-hydroxyethyl)pyrazole-3-carboxylic acid (4.2 mg, 0.021 mmol, 4.79% yield).

Step 4. To 2-[2-[tert-butyl(dimethyl)silyl]oxyethyl]-5-ethoxy-pyrazole-3-carboxylic acid (36 mg, 0.114 mmol) in acetonitrile (1 mL) was added NIS (28 mg, 125.9 mol). The reaction was stirred at 70° C. for 1.5 h. The mixture was then cooled and quenched with water, worked up with DCM and water (10 mL) and the layers were separated. The aqueous layer was extracted again with DCM (2×5 mL). The combined organic layer was washed with brine and dried over sodium sulfate. Flash column chromatography (automated system, 12 g silica, 0-25% Methanol in DCM) provided 2-[2-[tert-butyl(dimethyl)silyl]oxyethyl]-5-ethoxy-4-iodo-pyrazole-3-carboxylic acid (34 mg, 0.077 mmol, 67.44% yield).

Ex. 27 was prepared using General method F using the above intermediate in Step 4, and following Steps 1-5 and Step 7.

| | | MS m/z | 1H NMR (400 MHz, DMSO-d₆) |
|---|---|---|---|
| Ex # | Structure | [M + H]⁺ | δ ppm |
| 1 | 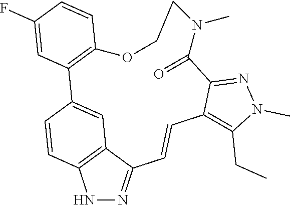 [SMILES: CCc1c2c(nn1C)C(=O)N(C)CCOc1ccc(F)cc1-c1ccc3[nH]nc(c3c1)/C=C/2] | 446.2 | 8.76 (s, 1 H), 7.52 (s, 2 H), 7.50 (d, J = 17.2 Hz, 1 H), 7.29 (dd, J = 9.6, 3.2 Hz, 1 H), 7.22 (dd, J = 7.2, 4.8 Hz, 1 H), 7.13 (d, J = 17.2 Hz, 1 H), 7.08-7.01 (m, 1 H), 4.65-4.43 (m, 3 H), 3.91 (s, 3 H), 3.45-3.34 (m, 1 H), 3.11 (s, 3 H), 3.06 (q, J = 15.2, 7.2 Hz, 2 H), 1.37 (t, J = 8.0 Hz, 3 H) |
| 2 | 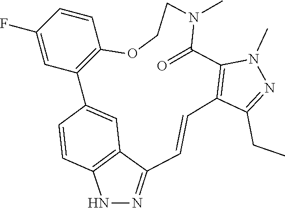 [SMILES: CCc1nn(C)c2c1/C=C/c1n[nH]c3ccc(cc13)-c1cc(F)ccc1OCCN(C)C2=O] | 446.2 | 8.77 (s, 1 H), 7.61 (s, 2 H), 7.52 (d, J = 17.2 Hz, 1 H), 7.31 (dd, J = 9.6, 3.2 Hz, 1 H), 7.23 (dd, J = 9.2, 4.4 Hz, 1 H), 7.17 (d, J = 17.2 Hz, 1 H), 7.08-7.03 (m, 1 H), 4.61-4.48 (m, 3 H), 3.85 (s, 3 H), 3.50-3.39 (m, 1 H), 3.16 (s, 3 H), 2.95 (q, J = 7.6 Hz, 2 H), 1.39 (t, J = 7.6 Hz, 3 H) |
| 3 | 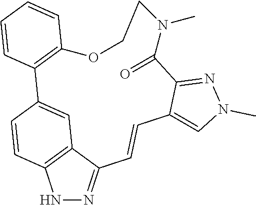 [SMILES: CN1CCOc2ccccc2-c2ccc3[nH]nc(c3c2)/C=C/c2cn(C)nc2C1=O] | 400.1 | 13.07-12.98 (m, 1H), 8.66 (s, 1H), 8.30 (s, 1H), 7.58 (s, 2H), 7.54 (d, J = 6.8 Hz, 1H), 7.34- 7.30 (m, 2H), 7.28-7.19 (m, 2H), 7.11-7.05 (m, 1H), 4.62- 4.34 (m, 4H), 3.90 (s, 3H), 3.00 (s, 3H) |
| 4 | 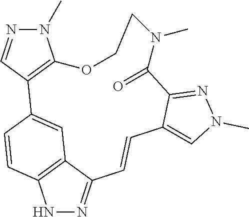 [SMILES: CN1CCOc2c(cnn2C)-c2ccc3[nH]nc(c3c2)/C=C/c2cn(C)nc2C1=O] | 404.2 | 13.01 (s, 1H), 8.46 (s, 1H), 8.31 (s, 1H), 7.94 (s, 1H), 7.72 (d, J = 8.8 Hz, 1H), 7.51 (d, J = 8.8 Hz, 1H), 7.22 (d, J = 1.2 Hz, 2H), 4.37-4.23 (m, 2H), 3.94-3.89 (m, 3H), 3.77 (s, 3H), 3.29 (s, 2H), 3.11 (s, 3H) |
| 5 | 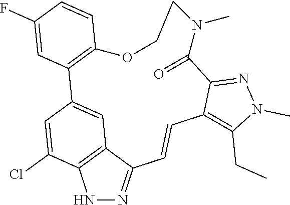 [SMILES: CCc1c2c(nn1C)C(=O)N(C)CCOc1ccc(F)cc1-c1cc(Cl)c3[nH]nc(c3c1)/C=C/2] | 480.1 | 7.49 (s, 1H), 7.38 (s, 1H), 7.17- 7.14 (m, 1H), 7.02-6.97 (m, 1H), 6.77 (dd, J = 2.0, 8.0 Hz, 1H), 6.58 (d, J = 8.0 Hz, 1H), 6.36 (s, 1H), 5.27 (br s, 1H), 4.22 (br d, J = 9.2 Hz, 1H), 3.94 (s, 3H), 3.91-3.83 (m, 2H), 3.39- 3.35 (m, 1H), 2.96-2.85 (m, 2H), 2.72 (s, 3H), 1.11 (br t, J = 7.2 Hz, 3H) |
| 6 | 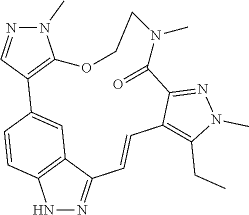 [SMILES: CCc1c2c(nn1C)C(=O)N(C)CCOc1c(cnn1C)-c1ccc3[nH]nc(c3c1)/C=C/2] | 432.0 | 13.06 (br s, 1H), 8.45 (s, 1H), 7.94 (s, 1H), 7.72 (dd, J = 1.2, 8.8 Hz, 1H), 7.52 (d, J = 8.8 Hz, 1H), 7.30 (d, J = 17.3 Hz, 1H), 7.08 (d, J = 17.3 Hz, 1H), 5.75 (s, 1H), 4.95 (br dd, J = 4.1, 14.9 Hz, 1H), 4.36-4.24 (m, 2H), 3.78 (d, J = 3.4 Hz, 6H), 3.39- 3.36 (m, 1H), 3.16 (s, 3H), 2.87 (q, J = 7.5 Hz., 2H), 2.07 (s, 1H), 1.31 (t, J = 7.5 Hz, 3H) |
| 7 | 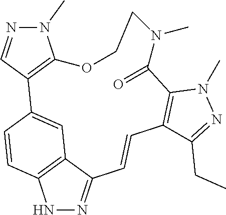 [SMILES: CCc1nn(C)c2c1/C=C/c1n[nH]c3ccc(cc13)-c1cnn(C)c1OCCN(C)C2=O] | 432.1 | 13.05 (br s, 1H), 8.42 (s, 1H), 7.94 (s, 1H), 7.72 (br d, J = 8.5 Hz, 1H), 7.52 (br d, J = 8.6 Hz, 1H), 7.29-7.17 (m, 1H), 7.03 (br d, J = 17.4 Hz, 1H), 4.80 (br d, J = 12.6 Hz, 1H), 4.37-4.19 (m, 2H), 3.85 (s, 3H), 3.77 (s, 3H), 3.27-3.18 (m, 1H), 3.09 (s, 3H), 3.03-2.93 (m, 2H), 1.28 (br t, J = 7.4 Hz, 3H) |
| 8 | 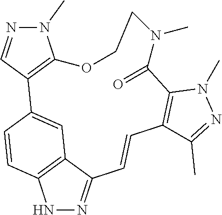 [SMILES: Cc1nn(C)c2c1/C=C/c1n[nH]c3ccc(cc13)-c1cnn(C)c1OCCN(C)C2=O] | 418.0 | 8.46 (s, 1H), 7.94 (s, 1H), 7.72 (dd, J = 2.0, 8.8 Hz, 1H), 7.52 (d, J = 8.8 Hz, 1H), 7.32 (d, J = 16.0 Hz, 1H), 7.11 (d, J = 16.0 Hz, 1H), 4.95 (br dd, J = 4.0, 12.0 Hz, 1H), 4.36-4.24 (m, 2H), 3.78 (s, 3H), 3.76 (s, 3H), 3.35 (br dd, J = 8.0, 12.0 Hz, 1H), 3.16 (s, 3H), 2.45 (s, 3H) |
| 9 | 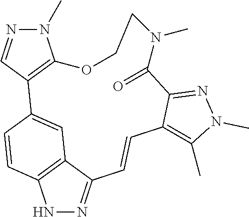 [SMILES: Cc1c2c(nn1C)C(=O)N(C)CCOc1c(cnn1C)-c1ccc3[nH]nc(c3c1)/C=C/2] | 418.0 | 13.03 (s, 1H), 8.43 (s, 1H), 7.93 (s, 1H), 7.72 (br d, J = 8.4 Hz, 1H), 7.52 (d, J = 8.8 Hz, 1H), 7.28-7.22 (m, 1H), 7.10-7.05 (m, 1H), 4.82 (br d, J = 12.0 Hz, 1H), 4.33-4.24 (m, 2H), 3.83 (s, 3H), 3.77 (s, 3H), 3.36 (br d, J = 8.4 Hz, 1H), 3.09 (s, 3H), 2.55 (s, 3H) |
| 10 | 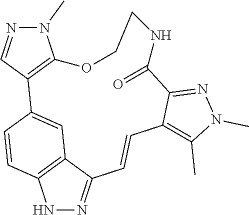 [SMILES: Cc1c2c(nn1C)C(=O)NCCOc1c(cnn1C)-c1ccc3[nH]nc(c3c1)/C=C/2] | 404.1 | 13.12-12.88 (m, 1H), 8.63 (t, J = 5.6 Hz, 1H), 8.58 (s, 1H), 7.93 (s, 1H), 7.71 (d, J = 9.6 Hz, 1H), 7.67 (d, J = 8.0 Hz, 1H), 7.51 (d, J = 8.4 Hz, 1H), 7.03 (d, J = 17.2 Hz, 1H), 4.18-4.13 (m, 2H), 3.83 (s, 3H), 3.77 (s, 1H), 3.75 (s, 3H), 3.70 (s, 1H), 2.54 (s, 3H) |
| 11 | 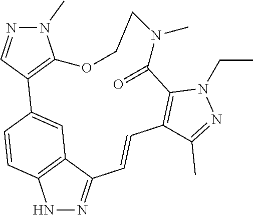 [SMILES: CCn1nc(C)c2c1C(=O)N(C)CCOc1c(cnn1C)-c1ccc3[nH]nc(c3c1)/C=C/2] | 431.9 | 13.06 (s, 1H), 8.45 (s, 1H), 7.94 (s, 1H), 7.72 (d, J = 8.8 Hz, 1H), 7.52 (d, J = 8.4 Hz, 1H), 7.31 (d, J = 17.2 Hz, 1H), 7.10 (d, J = 17.2 Hz, 1H), 4.97-4.89 (m, 1H), 4.36-4.25 (m, 2H), 4.15- 3.97 (m, 3H), 3.78 (s, 3H), 3.16 (s, 3H), 2.46 (s, 3H), 1.33 (t, J = 7.2 Hz, 3H) |
| 12 | 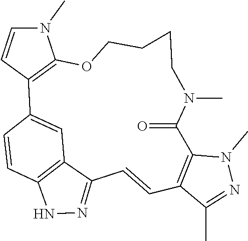 [SMILES: Cc1nn(C)c2c1/C=C/c1n[nH]c3ccc(cc13)-c1ccn(C)c1OCCCCN(C)C2=O] | 446.0 | 7.96 (s, 1H), 7.66 (s, 1H), 7.52- 7.48 (m, 1H), 7.47-7.42 (m, 1H), 7.33 (d, J = 16.4 Hz, 1H), 6.81 (d, J = 16.4 Hz, 1H), 4.44- 4.26 (m, 1H), 4.07-3.98 (m, 1H), 3.75 (s, 3H), 3.70 (s, 3H), 3.27 (s, 1H), 2.93 (s, 3H), 2.85- 2.77 (m, 1H), 2.48-2.36 (m, 1H), 2.30 (s, 3H), 1.99-1.83 (m, 1H), 1.71-1.57 (m, 2H) |
| 13 | 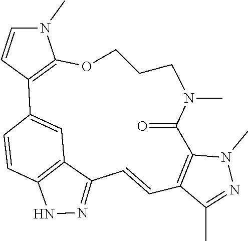 [SMILES: Cc1nn(C)c2c1/C=C/c1n[nH]c3ccc(cc13)-c1ccn(C)c1OCCCN(C)C2=O] | 432.2 | 8.17 (s, 1H), 7.71 (s, 1H), 7.63- 7.59 (m, 1H), 7.57-7.52 (m, 1H), 7.34-7.28 (m, 1H), 7.28- 7.22 (m, 1H), 4.72 (ddd, J = 4.0, 9.6, 13.6 Hz, 1H), 4.42 (td, J = 7.2, 10.0 Hz, 1H), 4.26 (td, J = 6.4, 10.0 Hz, 1H), 3.81 (d, J = 2.0 Hz, 6H), 3.24-3.12 (m, 1H), 3.06 (s, 3H), 2.54 (s, 3H), 2.43- 2.32 (m, 1H), 2.32-2.17 (m, 1H) |
| 14 | 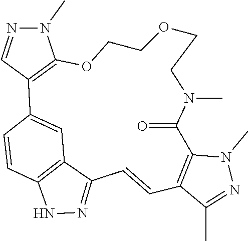 [SMILES: Cc1nn(C)c2c1/C=C/c1n[nH]c3ccc(cc13)-c1cnn(C)c1OCCOCCN(C)C2=O] | 462.1 | 13.36-12.61 (m, 1H), 8.26- 8.04 (m, 1H), 7.80-7.68 (m, 1H), 7.61-7.48 (m, 2H), 7.27- 7.11 (m, 1H), 6.99-6.88 (m, 1H), 4.14-4.04 (m, 2H), 3.88- 3.80 (m, 2H), 3.72 (d, J = 2.8 Hz, 3H), 3.68 (d, J = 6.4 Hz, 3H), 3.62-3.57 (m, 2H), 3.50- 3.38 (m, 2H), 3.18 (d, J = 9.6 Hz, 3H), 2.37-2.22 (m, 3H) |
| 15 | 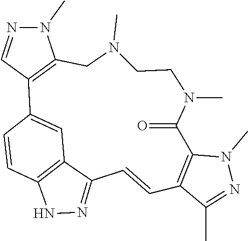 [SMILES: Cc1nn(C)c2c1/C=C/c1n[nH]c3ccc(cc13)-c1cnn(C)c1CN(C)CCN(C)C2=O] | 444.9 | 13.12 (s, 1H), 8.37 (s, 1H), 7.66 (s, 1H), 7.55 (d, J = 8.4 Hz, 1H), 7.43 (dd, J = 1.2, 8.4 Hz, 1H), 7.11 (s, 2H), 4.86-4.66 (m, 1H), 3.92 (s, 3H), 3.71 (s, 3H), 3.50 (s, 2H), 3.06-2.93 (m, 5H), 2.63- 2.54 (m, 1H), 2.40 (s, 3H), 2.25 (s, 3H) |
| 16 | | 448.0 | 13.35-12.83 (m, 1H), 8.47 (s, |
| | | | 1H), 7.94 (s, 1H), 7.72 (dd, J = |
| | | | 1.2, 8.8 Hz, 1H), 7.52 (d, J = 8.8 |
| | | | Hz, 1H), 7.34 (d, J = 16.0 Hz, |
| | | | 1H), 7.11 (d, J = 16.0 Hz, 1H), |
| | | | 4.96 (br dd, J = 4.4, 16.0 Hz, |
| | | | 1H), 4.35-4.24 (m, 2H), 4.18- |
| | | | 4.10 (m, 1H), 4.10-4.02 (m, |
| | | | 1H), 3.78 (s, 3H), 3.72-3.61 (m, |
| | | | 3H), 3.28 (br s, 1H), 3.17 (s, |
| | | | 3H), 2.47 (s, 3H) |
| 17 | 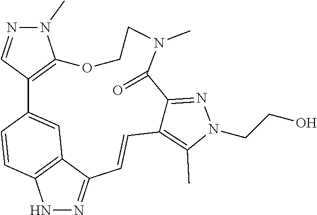 [SMILES: Cc1c2c(nn1CCO)C(=O)N(C)CCOc1c(cnn1C)-c1ccc3[nH]nc(c3c1)/C=C/2] | 448.0 | 13.09-12.95 (m, 1H), 8.43 (s, 1H), 7.94 (s, 1H), 7.74-7.70 (m, 1H), 7.52 (d, J = 9.2 Hz, 1H), 7.27-7.22 (m, 1H), 7.09-7.04 (m, 1H), 4.96 (t, J = 5.6 Hz, 1H), 4.80 (d, J = 11.6 Hz, 1H), 4.37- 4.25 (m, 2H), 4.18 (t, J = 5.6 Hz, 2H), 3.77 (s, 3H), 3.75 (d, J = 5.6 Hz, 2H), 3.47 - 3.38 (m, 1H), 3.10 (s, 3H), 2.57 (s, 3H) |
| 18 | 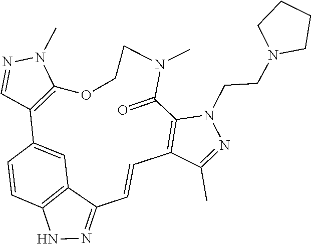 | 501.3 | 13.40-12.94 (m, 1H), 9.66 (br d, J = 1.2 Hz, 1H), 8.47 (s, 1H), 7.95 (s, 1H), 7.73 (dd, J = 1.0, 8.8 Hz, 1H), 7.54 (d, J = 8.8 Hz, 1H), 7.40-7.30 (m, 1H), 7.23- 7.13 (m, 1H), 5.00 (br dd, J = 5.6, 14.5 Hz, 1H), 4.50-4.40 (m, 2H), 4.39-4.32 (m, 1H), 4.31-4.23 (m, 1H), 3.79 (s, 3H), 3.72-3.54 (m, 5H), 3.31 (br d, J = 7.2 Hz, 3H), 3.18 (s, 3H), 3.12- 3.00 (m, 2H), 2.02 (br s, 2H), 1.91-1.80 (m, 2H) |
| 19 | 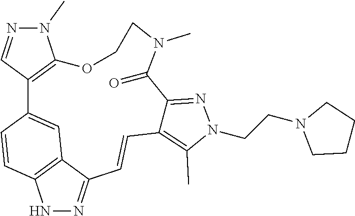 [SMILES: Cc1c2c(nn1CCN1CCCC1)C(=O)N(C)CCOc1c(cnn1C)-c1ccc3[nH]nc(c3c1)/C=C/2] | 501.1 | 10.09 (s, 1H), 8.43 (s, 1H), 7.94 (s, 1H), 7.73 (dd, J = 1.2, 9.2 Hz, 1H), 7.53 (d, J = 8.4 Hz, 1H), 7.27-7.21 (m, 1H), 7.15-7.10 (m, 1H), 4.80 (br d, J = 12.4 Hz, 1H), 4.54 (br t, J = 6.4 Hz, 3H), 4.33-4.25 (m, 2H), 3.77 (s, 3H), 3.69 (d, J = 4.0 Hz, 2H), 3.59 (br d, J = 3.6 Hz, 2H), 3.37 (br dd, J = 8.4, 13.6 Hz, 1H), 3.11 (s, 3H), 3.07 (br s, 2H), 2.62 (s, 3H), 2.08-1.98 (m, 2H), 1.92- 1.81 (m, 2H) |
| 20 | 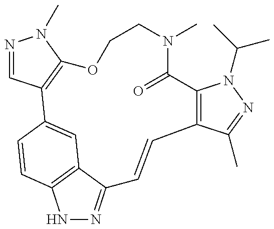 [SMILES: Cc1nn(C(C)C)c2c1/C=C/c1n[nH]c3ccc(cc13)-c1cnn(C)c1OCCN(C)C2=O] | 446.1 | 13.05 (s, 1H), 8.45 (s, 1H), 7.94 (s, 1H), 7.72 (dd, J = 1.2, 8.8 Hz, 1H), 7.52 (d, J = 8.8 Hz, 1H), 7.32-7.27 (m, 1H), 7.13-7.07 (m, 1H), 4.44 (quin, J = 6.4 Hz, 1H), 4.30 (d, J = 4.4 Hz, 2H), 3.78 (s, 1H), 3.79-3.76 (m, 1H), 3.44-3.37 (m, 2H), 3.35-3.34 (m, 3H), 3.16 (s, 3H), 2.47 (s, 3H), 1.43 (d, J = 6.4 Hz, 3H), 1.38 (d, J = 6.4 Hz, 3H) |
| 21 | 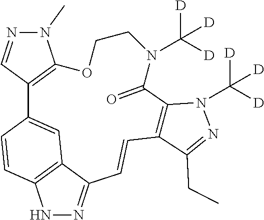 [SMILES: [2H]C([2H])([2H])N1CCOc2c(cnn2C)-c2ccc3[nH]nc(c3c2)/C=C/c2c(CC)nn(C([2H])([2H])[2H])c2C1=O] | 438.2 | 13.06 (s, 1H), 8.45 (s, 1H), 7.94 (s, 1H), 7.72 (d, J = 8.9 Hz, 1H), 7.52 (d, J = 8.8 Hz, 1H), 7.32 (s, 1H), 7.06 (s, 1H), 4.99-4.90 (m, 1H), 4.36-4.22 (m, 2H), 3.78 (s, 3H), 3.39-3.35 (m, 1H), 2.86 (q, J = 7.4 Hz, 2H), 1.31 (t, J = 7.5 Hz, 3H) |
| 22 | 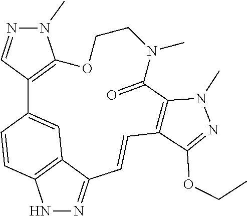 [SMILES: CCOc1nn(C)c2c1/C=C/c1n[nH]c3ccc(cc13)-c1cnn(C)c1OCCN(C)C2=O] | 448.2 | 8.46 (s, 1H), 7.94 (s, 1H), 7.71 (d, J = 8.8 Hz, 1H), 7.51 (d, J = 8.8 Hz, 1H), 7.36-7.27 (m, 1H), 7.25-7.15 (m, 1H), 5.00-4.84 (m, 1H), 4.39-4.25 (m, 4H), 3.78 (s, 3H), 3.69 (s, 3H), 3.42- 3.31 (m, 1H), 3.20 (s, 3H), 1.44 (t, J = 7.2 Hz, 3H) |
| 23 | 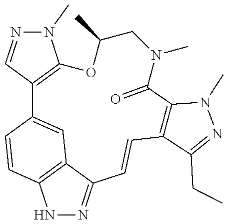 [SMILES: CCc1nn(C)c2c1/C=C/c1n[nH]c3ccc(cc13)-c1cnn(C)c1O[C@@H](C)CN(C)C2=O] | 446.5 | 8.73 (br s, 1 H), 7.97 (br s, 1 H), 7.68-7.76 (m, 1 H), 7.46-7.54 (m, 1 H), 7.42 (d, J = 17.15 Hz, 1 H), 7.09 (br d, J = 17.15 Hz, 1 H), 4.61-4.84 (m, 2 H), 3.70-3.80 (m, 6 H), 3.63 (br d, J = 15.78 Hz, 1 H), 3.35-3.36 (m, 3 H), 2.87 (q, J = 7.32 Hz, 2 H), 1.29-1.34 (m, 3 H), 1.23 (br s, 3 H) |
| 24 | 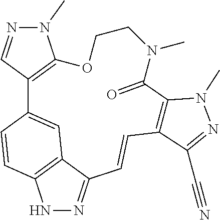 [SMILES: CN1CCOc2c(cnn2C)-c2ccc3[nH]nc(c3c2)/C=C/c2c(C#N)nn(C)c2C1=O] | 429.2 | 13.30 (s, 1 H), 8.46 (s, 1 H), 7.96 (s, 1 H), 7.76 (dd, J = 8.76, 1.10 Hz, 1 H), 7.61 (d, J = 17.25 Hz, 1 H), 7.56 (d, J = 8.76 Hz, 1 H), 7.28 (d, J = 16.97 Hz, 1 H), 4.91- 4.98 (m, 1 H), 4.31-4.38 (m, 2 H), 3.97 (s, 3 H), 3.79 (s, 3 H), 3.36-3.43 (m, 1 H), 3.19 (s, 3 H) |
| 25 | 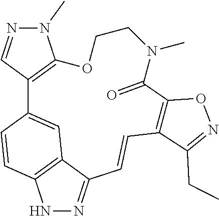 [SMILES: CCc1noc2c1/C=C/c1n[nH]c3ccc(cc13)-c1cnn(C)c1OCCN(C)C2=O] | 419.4 | 1H NMR (400 MHz, METHANOL-d4) δ ppm 8.56 (s, 1 H), 7.90 (s, 1 H), 7.76 (dd, J = 8.90, 1.51 Hz, 1 H), 7.57 (d, J = 8.76 Hz, 1 H), 7.42-7.47 (m, 1 H), 7.33-7.38 (m, 1 H), 5.02- 5.09 (m, 1 H), 4.40-4.47 (m, 1 H), 4.32-4.39 (m, 1 H), 3.84 (s, 3 H), 3.33-3.35 (m, 4 H), 3.07- 3.12 (m, 2 H), 1.47 (t, J = 7.39 Hz, 3 H) |
| 26 | 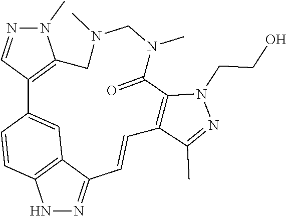 [SMILES: Cc1nn(CCO)c2c1/C=C/c1n[nH]c3ccc(cc13)-c1cnn(C)c1CN(C)CN(C)C2=O] | 475.1 | 1H NMR (400 MHz, METHANOL-d4) δ = 8.46 (s, 1H), 7.64 (s, 1H), 7.58-7.52 (m, 1H), 7.49-7.44 (m, 1H), 7.26 (s, 2H), 5.01-4.92 (m, 1H), 4.36- 4.25 (m, 1H), 4.17-4.08 (m, 1H), 3.98 (s, 3H), 3.85-3.77 (m, 2H), 3.55 (s, 2H), 3.20-3.10 (m, 1H), 3.08 (s, 3H), 3.00-2.91 (m, 1H), 2.64-2.56 (m, 1H), 2.52 (s, 3H), 2.37 (s, 3H) |
| 27 | 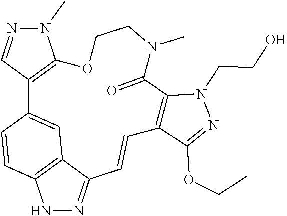 [SMILES: CCOc1nn(CCO)c2c1/C=C/c1n[nH]c3ccc(cc13)-c1cnn(C)c1OCCN(C)C2=O] | 478.3 | 12.98 (s, 1 H), 8.47 (s, 1 H), 7.94 (s, 1 H), 7.71 (dd, J = 8.90, 1.23 Hz, 1 H), 7.50 (d, J = 8.76 Hz, 1 H), 7.28-7.34 (m, 1 H), 7.19- 7.24 (m, 1 H), 4.93 (br dd, J = 14.78, 4.38 Hz, 2 H), 4.26- 4.38 (m, 4 H), 4.04-4.12 (m, 1 H), 3.97 (dt, J = 14.03, 3.66 Hz, 1 H), 3.78 (s, 3 H), 3.64 (br d, J = 4.38 Hz, 2 H), 3.21 (s, 3 H), 1.44 (t, J = 6.98 Hz, 4 H) |

### Screen Assays

### Biochemical Assay

### FLT3 and PIM Kinase Activity Evaluation at Reaction Biology Corporation

The inhibition activities against enzymatic kinases was evaluated using HotSpot assay platform (www.reactionbiology.com), a radiometric assay based on conventional filter-binding assays, that directly measures kinase catalytic activity toward a specific substrate (Anastassiadis T, et al. Comprehensive Assay of Kinase Catalytic Activity Reveals Features of Kinase Inhibitor Selectivity. Nat Biotechnol. 2011, 29:1039-45). Briefly, specific kinase/substrate pairs along with required cofactors were prepared in reaction buffer; 20 mM Hepes pH 7.5, 10 mM MgCl₂, 1 mM EGTA, 0.02% Brij35, 0.02 mg/ml BSA, 0.1 mM Na₃VO₄, 2 mM DTT, 1% DMSO. Compounds were delivered into the reaction, followed ˜20 minutes later by addition of a mixture of ATP (Sigma, St. Louis MO) and ³³P ATP (Perkin Elmer, Waltham MA) to a final concentration of 10 M. Reactions were carried out at room temperature for 120 min, followed by spotting of the reactions onto P81 ion exchange filter paper (Whatman Inc., Piscataway, NJ). Unbound phosphate was removed by extensive washing of filters in 0.75% phosphoric acid. After subtraction of background derived from control reactions containing inactive enzyme, kinase activity data was expressed as the percent remaining kinase activity in test samples compared to vehicle (dimethyl sulfoxide) reactions. IC₅₀ values and curve fits were obtained using Prism (GraphPad Software).

TABLE 1
Inhibition of kinase activities of FLT3 and FLT3-ITD

| | FLT3 | | PIM1 | PIM2 | PIM3 |
|---|---|---|---|---|---|
| | IC₅₀ | FLT3-ITD | IC₅₀ | IC₅₀ | IC₅₀ |
| Ex # | (nM) | IC₅₀ (nM) | (nM) | (nM) | (nM) |
| 6 | 0.088 | 0.348 | 2.65 | 5.68 | 0.115 |
| 7 | 0.247 | 0.537 | 6.72 | 14 | 0.221 |
| 8 | 0.217 | 0.487 | 1.64 | 8.17 | 0.0732 |
| 9 | 0.347 | 0.592 | 12.7 | 145 | 3.65 |
| 11 | 0.084 | 0.566 | | | |
| 19 | 0.564 | 1.052 | | | |
| 20 | 0.187 | 0.775 | | | |
| 22 | 0.093 | 0.527 | | | |
| 23 | 0.416 | 0.967 | | | |
| 24 | 0.23 | 0.475 | | | |

Evaluation of Binding Affinity with Wild Type and Mutant FLT3 Kinases Using KINOMEscan Assay at Eurofins DiscoveRx

For most kinases, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 111× stocks in 100% DMSO. Kds were determined using an 11-point 3-fold compound dilution series with three DMSO control points. All compounds for Kd measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1×PBS, 0.05% Tween 20). The beads were then re-suspended in elution buffer (1×PBS, 0.05% Tween 20, 0.5 μM nonbiotinylated affinity ligand) and incubated at room temperature with shaking for 30 minutes. The kinase concentration in the eluates was measured by qPCR. Binding constants (Kds) were calculated with a standard dose-response curve using a non-linear least square fit with the Levenberg-Marquardt algorithm.

TABLE 2
Binding affinity of Ex. 6 with wild type and mutant FLT3 kinases

| | | FLT3 | FLT3 | FLT3 | FLT3 | FLT3 | FLT3 |
|---|---|---|---|---|---|---|---|
| | FLT3 | (D835H) | (D835V) | (D835Y) | (ITD) | (ITD, D835V) | (ITD, F691L) |
| Ex # | K_{d} (nM) | K_{d} (nM) | K_{d} (nM) | K_{d} (nM) | K_{d} (nM) | K_{d} (nM) | K_{d} (nM) |
| 6 | 0.18 | 67 | 0.062 | 4.1 | 6.3 | 0.058 | 0.23 |

| | FLT3 | FLT3 | FLT3 | FLT3- |
|---|---|---|---|---|
| | (K663Q) | (N841I) | (R834Q) | autoinhibited |
| Ex. # | K_{d} (nM) | K_{d} (nM) | K_{d} (nM) | K_{d} (nM) |
| 6 | 16 | 2.2 | 0.89 | 9.1 |

### Inhibition of CLK1, CLK2, CLK3 and CLK4 Kinase Activity

Kinase protein and substrate were pre-diluted in the HEPES assay buffer (100 mM HEPES, pH 7.5, 0.01% Triton X-100, 0.1% BSA, 5 mM MgCl₂, 1 mM DTT, 10 μM Sodium Orthovanadate, 10 μM Beta-Glycerophosphate) dispensed into 384 well plate (5 μL per well). Control samples (0%-inhibition in the absence of inhibitor, DMSO only) and 100%-inhibition (in the absence of enzyme) were assembled in replicates of six and were used to calculate %-inhibition in the presence of compounds. Test compounds were added to the protein samples by acoustic dispensing (Labcyte Echo550). Concentration of DMSO was equalized to 1% in all samples. Reactions were initiated by addition of ATP by acoustic dispensing (Labcyte Echo550) and incubated according to assay specific incubation time. After incubation, 5 uL of Promega ADP-Glo reagent was added and incubated for 40 minutes. After 40 minutes, 10 uL of Promega kinase detection reagent was added. After 10 min of incubation with Kinase detection reagent, the luminescence was read on microplate reader (Biotek Synergy).

TABLE 3
Inhibition of CLK1, CLK2, CLK3 and CLK4 kinase activity

| | CLK1 | CLK2 | CLK3 | CLK4 |
|---|---|---|---|---|
| | IC₅₀ | IC₅₀ | IC₅₀ | IC₅₀ |
| Ex # | (nM) | (nM) | (nM) | (nM) |
| 6 | 0.134 | 0.165 | 5.87 | 0.446 |
| 15 | 0.157 | 0.161 | 2.77 | 0.53 |
| 16 | 0.299 | 0.16 | 10.4 | 0.758 |
| 22 | 0.355 | 0.253 | 9.47 | 0.668 |
| 23 | 0.843 | 0.225 | 5.53 | 1.39 |
| 24 | 0.415 | 0.67 | 92.9 | 0.593 |
| 26 | 0.233 | 0.386 | 31.1 | 0.605 |
| 27 | 1.55 | 1.11 | 108 | 3.03 |

### Cell Proliferation Assay

MV-4-11 cell line was purchased from ATCC and maintained in RPMI medium supplemented with 10% fetal bovine serum and 100 U/mL of penicillin/streptomycin. One thousand MV-4-11 cells/50 μl per well were seeded in white 384-well plate, followed by treatment with indicated compounds in a 11-dose dilution series. Cell proliferation was measured using CellTiter-Glo 2.0 luciferase-based ATP detection assay (Promega, Madison, WI) following the manufacturer's protocol. Plates were then read on a TECAN Sparks multimode microplate reader. Data was analyzed using GraphPad Prism 9 (GraphPad Software, San Diego, CA) to obtain IC₅₀ values.

TABLE 4
Inhibition of MV-4-11 cell proliferation.

| Ex # | IC₅₀ (nM) |
|---|---|
| 1 | 1162 |
| 2 | 352.7 |
| 4 | 21.2 |
| 5 | >10000 |
| 6 | 0.3 |
| 7 | 73.1 |
| 8 | <0.2 |
| 9 | ~1 |
| 10 | 864.9 |
| 11 | <0.2 |
| 12 | 148.4 |
| 13 | 15.5 |
| 14 | 384.5 |
| 15 | 4.6 |
| 16 | <1 |
| 17 | |
| 18 | 17.8 |
| 19 | 15.2 |
| 20 | 1.1 |
| 21 | <0.2 |
| 22 | <0.2 |
| 23 | 4.1 |
| 24 | 141.4 |
| 25 | 0.52 |
| 26 | 15 |
| 27 | 7.6 |

### Inhibition of Cell Proliferation in a Broad Panel of Cell Proliferation Assays

Ba/F3 cells were purchased from DSMZ. The FLT3-ITD gene was synthesized at GenScript and cloned into pCDH-CMV-MCS-EF1-Puro plasmid (System Biosciences, Inc). FLT3-ITD/D835V and FLT3-ITD/F691L cDNA clones were made at GenScript by PCR and confirmed by sequence. Ba/F3 FLT3-ITD, FLT3-ITD/D835V and FLT3-ITD/F691L cell lines were generated by infecting Ba/F3 cells with lentivirus containing FLT3-ITD, FLT3-ITD/D835V or FLT3-ITD/F691L genes. Stable cell lines were selected by puromycin treatment, followed by IL-3 withdrawal.

Cell proliferation assay was carried out in a panel of 108 cell lines. 1000 (attached cells) or 3000 (suspension cells) cells/100 μl per well were seeded in 96 well black plate with a clear bottom (Corning #3904). Compounds were added using Tecan D300e Digital Dispenser in 1:3 titration for 9 doses. Plates were incubated for 5 days at 37° C. and 5% CO2. Cell proliferation was measured using CellTiter-Glo 2.0 luciferase-based ATP detection assay (Promega, Madison, WI) at 50 μl per well following the manufacturer's protocol. Plates were then read on a TECAN Sparks multimode microplate reader. Data was analyzed using GraphPad Prism 9 (GraphPad Software, San Diego, CA) to obtain IC₅₀ values. IC₅₀ values were determined using Prism software (GraphPad Software, San Diego, CA).

TABLE 5
Anti-cell proliferation activity of Ex. 6 in a panel of 108 cell lines.

| | Cell Line | IC₅₀ | Tumor Type, | Source, | |
|---|---|---|---|---|---|
| | Name | (nM) | Disease | Catalog # | Culture Medium |
| 1 | BaF3 | 513 | murine IL-3 | DSMZ, ACC | 90% RPMI 1640 + |
| | | | dependent pro-B | 300 | 10% h.i. FBS + 1 |
| | | | cell line | | ng/mL mouse IL-3 |
| 2 | BaF3 FLT3- | 0.55 | murine pro-B | Engineered | 90% RPMI 1640 + |
| | ITD | | cell line | Cell line | 10% h.i. FBS |
| | | | expressing | | |
| | | | FLT3-ITD | | |
| 3 | BaF3 FLT3- | 1.3 | murine pro-B | Engineered | 90% RPMI 1640 + |
| | ITD/D835V | | cell line | Cell line | 10% h.i. FBS |
| | | | expressing | | |
| | | | FLT3- | | |
| | | | ITD/D835V | | |
| 4 | BaF3 FLT3- | 22.9 | murine pro-B | Engineered | 90% RPMI 1640 + |
| | ITD/F691L | | cell line | Cell line | 10% h.i. FBS |
| | | | expressing | | |
| | | | FLT3- | | |
| | | | ITD/F691L | | |
| 5 | MV-4-11 | 0.68 | Biphenotypic B- | ATCC, | 90% IMDM + 10% |
| | | | Myelomonocytic | CRL-591 | FBS |
| | | | Leukemia | | |
| 6 | MOLM14 | 2.26 | Acute myeloid | DSMZ, | 90% RPMI 1640 + |
| | | | leukemia | ACC 777 | 10% h.i. FBS |
| 7 | MOLM13 | 2.39 | Acute myeloid | DSMZ, | 90% RPMI 1640 + |
| | | | leukemia | ACC 554 | 10% h.i. FBS |
| 8 | KASUMI-4 | 11.16 | Chronic | ATCC, | 80% RPMI1640 + |
| | | | Myeloblastic | CRL-2726 | 20% h.i. FBS + 10 |
| | | | Leukemia | | ng/ml GM-CSF |
| 9 | IMR-32 | 11.16 | Neuroblastoma | ATCC, | 90% DMEM + 10% |
| | | | | CCL-127 | h.i. FBS |
| 10 | KASUMI-1 | 13.98 | Acute | ATCC, | 90% RPMI 1640 + |
| | | | myeloblastic | CRL-2724 | 10% h.i. FBS |
| | | | leukemia | | |
| 11 | KP-N-RT- | 15.07 | Neuroblastoma | JCRB, | 90% RPMI 1640 + |
| | BM-1 | | | IFO50432 | 10% h.i. FBS |
| 12 | UCSD- | 15.47 | Acute myeloid | DSMZ, ACC | 80% RPMI 1640 + |
| | AML1 | | leukemia | 691 | 20% h.i. FBS + 10 |
| | | | | | ng/ml GM-CSF |
| 13 | SW403 | 18.04 | Colorectal | ATCC, | 90% Leibovitz's L-15 |
| | | | adenocarcinoma | CCL-230 | Medium + 10% h.i. |
| | | | | | FBS |
| 14 | EOL-1 | 18.92 | Chronic | Sigma Aldrich, | 90% RPMI 1640 + |
| | | | eosinophilic | 94042252 | 10% h.i. FBS |
| | | | leukemia | | |
| 15 | JeKo-1 | 20.63 | Mantle cell | ATCC, | 80% RPMI 1640 + |
| | | | lymphoma | CRL-3006 | 20% h.i. FBS |
| 16 | N87 | 20.83 | Gastric | ATCC, | 90% RPMI 1640 + |
| | | | carcinoma | CRL-5822 | 10% h.i. FBS |
| 17 | SK-N-SH | 21.72 | Neuroblastoma | ATCC, HTB-11 | 90% DMEM + 10% |
| | | | | | h.i. FBS |
| 18 | HNT-34 | 22.31 | Acute myeloid | DSMZ, ACC | 90% RPMI 1640 + |
| | | | leukemia | 600 | 10% h.i. FBS |
| 19 | SKNO-1 | 23.34 | Acute myeloid | DSMZ, ACC | 90% RPMI 1640 + |
| | | | leukemia | 690 | 10% h.i. FBS + 10 |
| | | | | | ng/ml GM-CSF |
| 20 | AU565 | 25.46 | Breast | ATCC, CRL- | 90% RPMI 1640 + |
| | | | adenocarcinoma | 2351 | 10% h.i. FBS |
| 21 | CHP-212 | 27.44 | Neuroblastoma | ATCC, CRL- | 90% DMEM + 10% |
| | | | | 2273 | h.i. FBS |
| 22 | SU-DHL-6 | 28.37 | Diffuse Large B | ATCC, CRL- | 90% RPMI 1640 + |
| | | | Cell Lymphoma | 2959 | 10% h.i. FBS |
| 23 | LS513 | 30.11 | Colorectal | ATCC, CRL- | 90% RPMI 1640 + |
| | | | carcinoma | 2134 | 10% h.i. FBS |
| 24 | Toledo | 34.58 | Diffuse Large B | ATCC, CRL- | 90% RPMI 1640 + |
| | | | Cell Lymphoma | 2631 | 10% h.i. FBS |
| 25 | MFM-223 | 36.75 | Breast carcinoma | Sigma, | 90% DMEM + 10% |
| | | | | 98050130-1VL | h.i. FBS |
| 26 | SK-BR-3 | 38.77 | Breast | ATCC, HTB-30 | 90% McCoy's 5a |
| | | | adenocarcinoma | | Medium Modified + |
| | | | | | 10% hi.i. FBS |
| 27 | HCC78 | 42.71 | Non-small cell | DSMZ, ACC | 90% RPMI 1640 + |
| | | | lung carcinoma | 563 | 10% h.i. FBS |
| 28 | KASUMI-3 | 43.32 | Acute | ATCC, CRL- | 80% RPMI 1640 + |
| | | | myeloblastic | 2725 | 20% h.i. FBS |
| | | | leukemia | | |
| 29 | NB-1 | 45.04 | Neuroblastoma | JCRB, | 90% RPMI 1640 + |
| | | | | JCRB0621 | 10% h.i. FBS |
| 30 | PA-1 | 47.97 | Ovary | ATCC, CRL- | 90% DMEM + 10% |
| | | | Teratocarcinoma | 1572 | h.i. FBS |
| 31 | DLD1 | 48.70 | Colorectal | ATCC, CCL- | 90% RPMI 1640 + |
| | | | adenocarcinoma | 221 | 10% h.i. FBS |
| 32 | Kelly | 51.47 | Neuroblastoma | DSMZ, ACC | 90% RPMI 1640 + |
| | | | | 355 | 10% h.i. FBS |
| 33 | MIAPaCa-2 | 53.25 | Pancreatic | ATCC, CRL- | 90% DMEM + 10% |
| | | | carcinoma | 1420 | h.i. FBS |
| 34 | TF-1 | 57.62 | Erythroleukemia | ATCC, CRL- | 90% RPMI 1640 + |
| | | | | 2003 | 10% h.i. FBS |
| 35 | HCT116 | 60.20 | Colorectal | Sigma, | 90% McCoy's 5a |
| | | | adenocarcinoma | 91091005 | Medium Modified + |
| | | | | | 10% hi.i. FBS |
| 36 | KCL22S | 61.19 | Chronic myeloid | ATCC, CRL- | 90% RPMI 1640 + |
| | | | leukemia at blast | 3349 | 10% h.i. FBS |
| | | | crisis | | |
| 37 | DU4475 | 61.44 | Breast carcinoma | ATCC, HTB- | 90% RPMI 1640 + |
| | | | | 123 | 10% h.i. FBS |
| 38 | TOV-112D | 62.54 | Endometrioid | ATCC, CRL- | 90% RPMI 1640 + |
| | | | ovarian cancer | 11731 | 10% h.i. FBS |
| 39 | KCL22R | 64.75 | Chronic | ATCC, CRL- | 90% RPMI 1640 + |
| | | | Myelogenous | 3350 | 10% h.i. FBS |
| | | | Leukemia | | |
| 40 | A549 | 67.73 | Lung carcinoma, | ATCC, CCL- | 90% RPMI 1640 + |
| | | | NSCLC | 185 | 10% h.i. FBS |
| 41 | Ovcar3 | 70.68 | Ovarian | ATCC. HTB- | 80% RPMI 1640 + |
| | | | adenocarcinoma | 161 | 20% h.i. FBS |
| 42 | HCC827 | 70.90 | Lung | ATCC, CRL- | 90% RPMI 1640 + |
| | | | adenocarcinoma | 2868 | 10% h.i. FBS |
| | | | NSCLC | | |
| 43 | 22RV1 | 73.23 | Prostate | ATCC, CRL- | 90% RPMI 1640 + |
| | | | carcinoma | 2505 | 10% h.i. FBS |
| 44 | H2122 | 73.72 | Lung | ATCC, CRL- | 90% RPMI 1640 + |
| | | | adenocarcinoma | 5985 | 10% h.i. FBS |
| | | | NSCLC | | |
| 45 | H526 | 75.49 | Small cell lung | ATCC, CRL- | 90% RPMI 1640 + |
| | | | cancer | 5811 | 10% h.i. FBS |
| 46 | MCF7 | 75.89 | Breast | ATCC, HTB-22 | 90% DMEM + 10% |
| | | | adenocarcinoma | | h.i. FBS |
| 47 | SW620 | 76.50 | Colorectal | ATCC, CCL- | 90% Leibovitz's L-15 |
| | | | adenocarcinoma | 227 | Medium + 10% h.i. |
| | | | | | FBS |
| 48 | SK-N-AS | 77.23 | Neuroblastoma | ATCC, CRL- | 90% DMEM + 10% |
| | | | | 2137 | h.i. FBS |
| 49 | HPAC | 79.08 | Pancreatic | ATCC, CRL- | DMEM:F12K + |
| | | | carcinoma | 2119 | insulin + EGF + 5% |
| | | | | | FBS |
| 50 | MOLM-1 | 80.45 | Chronic myeloid | DSMZ, ACC | 80% RPMI 1640 + |
| | | | leukemia | 720 | 20% h.i. FBS |
| 51 | H1573 | 80.64 | Lung | ATCC, CRL- | 90% RPMI 1640 + |
| | | | adenocarcinoma | 5877 | 10% h.i. FBS |
| | | | NSCLC | | |
| 52 | PANC0504 | 82.22 | Pancreatic | ATCC, CRL- | 90% RPMI 1640 + |
| | | | carcinoma | 2557 | 10% h.i. FBS |
| 53 | LS180 | 82.71 | Colorectal | ATCC, CL-187 | 90% DMEM + 10% |
| | | | adenocarcinoma | | h.i. FBS |
| 54 | HCC1419 | 83.80 | Breast | ATCC, CRL- | 90% RPMI 1640 + |
| | | | carcinoma, | 2326 | 10% h.i. FBS |
| | | | TNBC | | |
| 55 | COLO320 | 85.17 | Colorectal | ATCC, CCL- | 90% RPMI 1640 + |
| | | | adenocarcinoma | 220 | 10% h.i. FBS |
| 56 | HCC1806 | 85.87 | Breast | ATCC, CRL- | 90% RPMI 1640 + |
| | | | carcinoma, | 2335 | 10% h.i. FBS |
| | | | TNBC | | |
| 57 | H1975 | 87.17 | Lung | ATCC, CRL- | 90% RPMI 1640 + |
| | | | adenocarcinoma | 5908 | 10% h.i. FBS |
| | | | NSCLC | | |
| 58 | AGS | 87.94 | Gastric | ATCC, CRL- | 90% F-12K + 10% |
| | | | adenocarcinoma | 1739 | h.i. FBS |
| 59 | H23 | 88.82 | Lung | ATCC, CRL- | 90% RPMI 1640 + |
| | | | adenocarcinoma | 5800 | 10% h.i. FBS |
| | | | NSCLC | | |
| 60 | U87 MG | 89.20 | Glioblastoma | ATCC, HTB-14 | 90% DMEM + 10% |
| | | | | | h.i. FBS |
| 61 | SW480 | 90.46 | Colorectal | ATCC, CCL- | 90% Leibovitz's L-15 |
| | | | adenocarcinoma | 228 | Medium + 10% h.i. |
| | | | | | FBS |
| 62 | SNU-16 | 90.47 | Gastric | ATCC, CRL- | 90% RPMI 1640 + |
| | | | carcinoma | 5974 | 10% h.i. FBS |
| 63 | H441 | 91.12 | Lung | ATCC, HTB- | 90% RPMI 1640 + |
| | | | adenocarcinoma | 174 | 10% h.i. FBS |
| | | | NSCLC | | |
| 64 | 769-P | 91.60 | Renal cell | ATCC, CRL- | 90% RPMI 1640 + |
| | | | adenocarcinoma | 1933 | 10% h.i. FBS |
| 65 | COR-L23 | 98.44 | Large cell lung | DSMZ, ACC | 90% RPMI 1640 + |
| | | | carcinoma | 882 | 10% h.i. FBS |
| 66 | HGC-27 | 100.10 | Gastric | Millipore | 90% EMEM + 2 mM |
| | | | carcinoma | Sigma, | Glutamine + 1% Non |
| | | | | CB_94042256 | Essential Amino |
| | | | | | Acids + 10% h.i. FBS |
| 68 | H69 | 100.30 | Lung small cell | ATCC, HTB- | 90% RPMI 1640 + |
| | | | carcinoma | 119 | 10% h.i. FBS |
| 69 | H727 | 100.50 | Bronchial lung | ATCC, CRL- | 90% RPMI 1640 + |
| | | | carcinoid | 5815 | 10% h.i. FBS |
| 70 | SW1990 | 104.10 | Pancreatic | ATCC, CRL- | 90% Leibovitz's L-15 |
| | | | adenocarcinoma | 2172 | Medium + 10% h.i. |
| | | | | | FBS |
| 71 | H211 | 104.80 | Small cell lung | ATCC, CRL- | 90% RPMI 1640 + |
| | | | carcinoma | 5824 | 10% h.i. FBS |
| 72 | THP-1 | 106.90 | Acute monocytic | ATCC, TIB- | 90% RPMI 1640 + |
| | | | leukemia | 202 | 10% h.i. FBS + |
| | | | | | 0.05 mM BME |
| 73 | H446 | 112.20 | Small cell lung | ATCC, HTB- | 90% RPMI 1640 + |
| | | | carcinoma | 171 | 10% h.i. FBS |
| 74 | T47D | 114.26 | Mammary gland | ATCC, HTB- | 90% RPMI 1640 + |
| | | | ductal carcinoma | 133 | 0.2 units/mL bovine |
| | | | | | insulin + 10% h.i. |
| | | | | | FBS |
| 75 | H1688 | 115.50 | Small cell lung | ATCC, CCL- | 90% RPMI 1640 + |
| | | | carcinoma | 257 | 10% h.i. FBS |
| 76 | SKOV3 | 116.90 | Ovarian | ATCC, HTB-77 | 90% McCoy's 5a |
| | | | adenocarcinoma | | Medium Modified + |
| | | | | | 10% h.i. FBS |
| 77 | H2444 | 117.10 | Lung | ATCC, CRL- | 90% RPMI 1640 + |
| | | | adenocarcinoma | 5945 | 10% h.i. FBS |
| | | | NSCLC | | |
| 78 | H2228 | 117.93 | Lung | ATCC, CRL- | 90% RPMI 1640 + |
| | | | adenocarcinoma, | 5935 | 10% h.i. FBS |
| | | | NSCLC | | |
| 79 | H1792 | 120.53 | Lung | ATCC, CRL- | 90% RPMI 1640 + |
| | | | adenocarcinoma | 5895 | 10% h.i. FBS |
| | | | NSCLC | | |
| 80 | GP2D | 121.30 | Colon | Millipore | DMEM + 2 mM |
| | | | adenocarcinoma | Sigma, | Glutamine + 10% h.i. |
| | | | | CB_950914 | FBS |
| 81 | PANC0203 | 126.10 | Pancreatic | ATCC, CRL- | 85% RPMI 1640 + |
| | | | adenocarcinoma | 2553 | 10 U/mL human |
| | | | | | recombinatant insulin + |
| | | | | | 15% h.i. FBS |
| 82 | LNCaP | 135.90 | Prostate | ATCC, CRL- | 90% RPMI 1640 + |
| | | | carcinoma | 1740 | 10% h.i. FBS |
| 83 | 786-O | 138.40 | Renal cell | ATCC, CRL- | 90% RPMI 1640 + |
| | | | adenocarcinoma | 1932 | 10% h.i. FBS |
| 84 | H2087 | 141.20 | Lung | ATCC, CRL- | 95% RPMI 1640 + |
| | | | adenocarcinoma | 5922 | 5% h.i. FBS |
| | | | NSCLC | | |
| 85 | Caki-2 | 141.70 | Kidney clear cell | ATCC, HTB-47 | 90% McCoy's 5a |
| | | | carcinoma | | Medium Modified + |
| | | | | | 10% h.i. FBS |
| 86 | ZR-75-1 | 144.00 | Mammary gland | ATCC, CRL- | 90% RPMI 1640 + |
| | | | ductal carcinoma | 1500 | 10% h.i. FBS |
| 87 | H358 | 147.60 | Bronchioalveolar | ATCC, CRL- | 90% RPMI 1640 + |
| | | | carcinoma | 5807 | 10% h.i. FBS |
| | | | NSCLC | | |
| 88 | HCC1569 | 151.30 | Mammary gland | ATCC, CRL- | 90% RPMI 1640 + |
| | | | metaplastic | 2330 | 10% h.i. FBS |
| | | | carcinoma | | |
| 89 | KG-1 | 152.80 | Acute | ATCC, CCL- | 80% Iscove's |
| | | | myelogenous | 246 | Modified Dulbecco's |
| | | | leukemia | | Medium + 20% h.i. |
| | | | | | FBS |
| 90 | KATOIII | 158.50 | Gastric | ATCC, HTB- | 80% Iscove's |
| | | | carcinoma | 103 | Modified Dulbecco's |
| | | | | | Medium + 20% h.i. |
| | | | | | FBS |
| 91 | HCC38 | 160.80 | Mammary gland | ATCC, CRL- | 90% RPMI 1640 + |
| | | | ductal carcinoma | 2314 | 10% h.i. FBS |
| 92 | PC-9 | 163.79 | Lung | Millipore | 90% RPMI 1640 + |
| | | | adenocarcinoma | Sigma, | 2 mM Glutamine + |
| | | | NSCLC | CB_90071810 | 10% h.i. FBS |
| 93 | SK-LU-1 | 165.90 | Lung | ATCC, HTB-57 | 90% Eagle's |
| | | | adenocarcinoma | | Minimum Essential |
| | | | NSCLC | | Medium + 10% h.i. |
| | | | | | FBS |
| 94 | A-498 | 169.20 | Kidney | ATCC, HTB-44 | 90% Eagle's |
| | | | carcinoma | | Minimum Essential |
| | | | | | Medium + 10% h.i. |
| | | | | | FBS |
| 95 | U138-MG | 169.50 | Brain | ATCC, HTB-16 | 90% Eagle's |
| | | | glioblastaoma | | Minimum Essential |
| | | | | | Medium + 10% h.i. |
| | | | | | FBS |
| 96 | JPC3 | 170.80 | Lung carcinoma | JCRB, | 90% RPMI 1640 + |
| | | | | JCRB0077 | 10% h.i. FBS |
| 97 | H1930 | 180.70 | Lung carcinoma | ATCC, CRL- | 90% RPMI 1640 + |
| | | | small cell lung | 5906 | 10% h.i. FBS |
| | | | cancer | | |
| 98 | Hs746T | 183.40 | Gastric | ATCC, HTB- | 90% DMEM + 10% |
| | | | carcinoma | 135 | h.i. FBS |
| 99 | PANC1 | 212.60 | Pancreatic ductal | ATCC, CRL- | 90% DMEM + 10% |
| | | | epithelioid | 1469 | h.i. FBS |
| | | | carcinoma | | |
| 100 | CT26 | 214.59 | Murine colon | ATCC, CRL- | 90% RPMI 1640 + |
| | | | carcinoma | 2638 | 10% h.i. FBS |
| | | | fibroblasts | | |
| 101 | MC38 | 216.94 | Murine colon | Creative | 90% DMEM + 10% |
| | | | adenocarcinoma | Bioarray, | h.i. FBS |
| | | | | CSC6983J | |
| 102 | PANC0403 | 220.00 | Pancreatic | ATCC, CRL- | 85% RPMI 1640 + |
| | | | adenocarcinoma | 2555 | 20 U/mL human |
| | | | | | recombinant insulin + |
| | | | | | 15% h.i. FBS |
| 103 | ASPC1 | 240.85 | Pancreatic | ATCC, CRL- | 90% RPMI 1640 + |
| | | | adenocarcinoma | 1682 | 10% h.i. FBS |
| 104 | BT-474 | 277.60 | Mammary gland | ATCC, HTB-20 | Hybri-Care Medium |
| | | | ductal carcinoma | | reconstituted in 1 L |
| | | | | | water and 1.5 g/L of |
| | | | | | Sodium bicarbonate. |
| | | | | | 10% h.i. FBS |
| 105 | U118-MG | 310.00 | Brain | ATCC, HTB-15 | 90% DMEM + 10% |
| | | | glioblastoma | | h.i. FBS |
| 106 | H2291 | 361.90 | Lung | ATCC, CRL- | 90% RPMI 1640 + |
| | | | adenocarcinoma | 5939 | 10% h.i. FBS |
| | | | NSCLC | | |
| 107 | CAPAN2 | 365.20 | Pancreatic | ATCC, HTB-80 | McCoy's 5a Medium |
| | | | adenocarcinoma | | Modified + 10% h.i. |
| | | | | | FBS |
| 108 | SUIT-2 | 393.00 | Pancreatic | JCRB, | 90% RPMI 1640 + |
| | | | tubular | JCRB1094 | 10% h.i. FBS |
| | | | adenocarcinoma | | |
| 109 | BJ | 516.16 | Skin fibroblast | ATCC, CRL- | 90% EMEM + 10% |
| | | | | 2522 | h.i. FBS |

### Inhibition of Kinase Phosphorylation in MV-4-11 and MOLM13 Cell Lines

Inhibition of kinase phosphorylation assay was performed by Western Blot using MV-4-11 and MOLM13 cell lines. Cells were plated in 24 well plates at 1 million cells per well and then treated with compounds at different concentrations, starting at 100 nM, 1:4 titration with DMSO as control. After 4 hour-incubation, cells were washed once with ice-cold PBS, and lysed in RIPA lysis buffer with protease and phosphatase inhibitors. The cell lysates were then sonicated and spun down, and 40 μg of proteins were separated by sodium dodecyl sulfate electrophoresis on a 4 to 12% Bis-Tris polyacrylamide gel and transferred onto nitrocellulose membranes using Bio-Rad Trans-Blot Turbo Transfer System (Bio-Rad). Blocking was performed using 3% BSA or 5% Non-fat dry milk (Fisher Scientific) in TBST (Tris-buffered saline containing 0.05% of Tween 20) (Bio-Rad Laboratories, Inc.). The membranes were incubated overnight with anti-phospho-FLT3 (Y589/Y591) (CST #60413), anti-phospho-STAT5 (Y694) (CST #4322), anti-Phospho-p44/42 MAPK (Erk1/2) (T202/Y204) (CST #9101), anti-Phospho-S6 Ribosomal Protein (S235/236) (CST #4858) and anti-β-Actin (CST #8457) each diluted 1000 times in 3% BSA in TBST. Membranes were washed with TBST and then incubated at room temperature for 1 hour with HRP-labeled anti-rabbit IgG polyclonal antibody (CST #7074) diluted 1000 times in 3% BSA. After TBST washing, blots were developed with a chemiluminescence system (SuperSignal West Femto Maximum Sensitivity Substrate, Pierce #34095). Luminescence was detected by LI-COR Odyssey M imaging system and luminescence intensity were quantified using Empiria Studio 2.2 software. Data was analyzed using GraphPad Prism 9 (GraphPad Software, San Diego, CA) to obtain IC₅₀ values.

TABLE 7
Inhibition of kinase phosphorylation with Ex. 6
and Gilteritinib in MV-4-11 and MOLM-13 cells.

| | |
|---|---|
| MV-4-11 IC₅₀ (nM) | MOLM-13 IC₅₀ (nM) |

| | Gilteritinib | Ex. 6 | Gilteritinib | Ex. 6 |
|---|---|---|---|---|
| pFLT3 | 0.84 | 0.16 | 1.03 | 0.32 |
| pSTAT5 | 1.75 | 0.60 | 2.23 | 0.72 |
| pERK | 1.63 | 2.85 | 1.31 | 4.11 |
| pS6 | 0.92 | 0.41 | 7.91 | 2.47 |

Modulation of alternative splicing in MV-4-11 and MOLM-13 cells with Ex. 6.

Alternative splicing ratio of RNA transcripts was measured by qPCR in MV-4-11 and MOLM-13 cell lines. Cells were plated in 24 well plates at 1 million cells per well and then treated with compounds at different concentrations, with DMSO only as control. After 8-hour-incubation, cells were harvested and washed once with ice-cold PBS, and lysed in RNA lysis buffer (Invitrogen). Total RNA was extracted using the RNA Purification Kit (Invitrogen). 2 μg of RNA was used for cDNA synthesis using cDNA synthesis kit (ThermoFisher) and 20 ng of cDNA was used for qPCR using PROFLEX 96-Well PCR System (ThermoFisher).

Primers with the following sequences were used for the measurement:
BCLAF1 exon10-12/exon 10-11 (TaqMan, IDT)

BCLAF1 (ex10-11) F:
(SEQ ID NO: 1)
CAGGAGTTAGCCGACCACG
BCLAF1 (ex10-11) R:
(SEQ ID NO: 2)
GTTTGGACCAGTATTTGTCCCAG
BCLAF1 (ex10-11) probe:
(SEQ ID NO: 3)
AACCTTTTTTCGAATTAGAGGCA
BCLAF1 (ex10-12) F:
(SEQ ID NO: 4)
TGCAGGAGTTAGCCGACCAC
BCLAF1 (ex10-12) R:
(SEQ ID NO: 5)
TTGGCCCAATAATCCACACC
BCLAF1 (ex10-12) probe:
(SEQ ID NO: 6)
AACCTTTCATGACGACAGAG

PRS6KB1 exon6-8/exon 6-7 (TaqMan, IDT)

S6K (ex6-7) F:
(SEQ ID NO: 7)
TTATGCAGTTAGAAAGAGAGGGAATATTT
S6K (ex6-7) R:
(SEQ ID NO: 8)
CCCCAAAGCCATGGAGATTT
S6K (ex6-7) probe:
(SEQ ID NO: 9)
TGGAAGACACTGCCTGCTTTTACTTGG
S6K (ex6-8) F:
(SEQ ID NO: 10)
TTATGCAGTTAGAAAGAGAGGGAATATTT
S6K (ex6-8) R:
(SEQ ID NO: 11)
GATTCTTTGCATAGTCCAAAGTCTGT
S6K (ex6-8) probe:
(SEQ ID NO: 12)
TGGAAGACACTGCCTGGTCATGTGAAA

BCLxS/xL (SYBR Green, IDT)

BCLxS F:
(SEQ ID NO: 13)
GAGCTTTGAACAGGATACTTTTGTG
BCLxS R:
(SEQ ID NO: 14)
GAAGAGTGAGCCCAGCAGAA
BCLxL F:
(SEQ ID NO: 15)
GATGGCCACTTACCTGAATGA
BCLxL R:
(SEQ ID NO: 16)
TGCTGCATTGTTCCCATAGA

TABLE 8
Modulation of alternative splicing of BCLAF1, SK6K and
BCL-x in MOLM-13 cell line with Ex. 6 by qPCR method.

| Alternative Splicing | | BCLAF1 Ex. | S6K Ex. | |
|---|---|---|---|---|
| Ratio in MOLM-13 | | 10-12/Ex. 10-11 | 6-8/Ex. 6-7 | BCL-xS/xL |
| DMSO | | 1.00 | 1.02 | 1.00 |
| 30 | nM (Ex. 6) | 1.80 | 1.60 | 1.51 |
| 100 | nM (Ex. 6) | 5.76 | 2.71 | 3.54 |
| 300 | nM ((Ex. 6) | 6.89 | 8.08 | 4.20 |

TABLE 9
Modulation of alternative splicing of BCL-x in
MV-4-11 cell line with Ex. 6 by qPCR method.

| Alternative Splicing | BCL-xS/xL | BCL-xS/xL |
|---|---|---|
| Ratio in MV-4-11 | 6-hour | 24-hour |
| DMSO | 1 | 1 |

| | | | |
|---|---|---|---|
| 2 | nM (Ex. 6) | 0.7 | 0.6 |
| 10 | nM (Ex. 6) | 1.1 | 0.9 |
| 50 | nM (Ex. 6) | 1.4 | 1.3 |
| 250 | nM (Ex. 6) | 3.7 | 7.3 |

### Alternative Splicing Events by RNAseq in MOLM-13

Alternative splicing events were measured by RNAseq in MOLM-13 cell line. Cells were plated in 24 well plates at 1 million cells per well and then treated with compounds at different concentrations, with DMSO as control. After 8 hour-incubation, cells were harvested and washed once with ice-cold PBS, and lysed in RNA lysis buffer (Invitrogen). Total RNA was extracted using the RNA Purification Kit (Invitrogen). RNAseq was performed by Biomiga (San Diego).

TABLE 10
Alternative splicing events by RNAseq in MOLM-13

| AS Events | | A3′SS | A5′SS | MXE | RI | SE |
|---|---|---|---|---|---|---|
| 30 | nM (Ex. 6) | 371 | 289 | 187 | 536 | 2054 |
| 100 | nM (Ex. 6) | 559 | 443 | 325 | 841 | 4568 |
| 300 | nM (Ex. 6) | 722 | 590 | 355 | 1184 | 5093 |

A3′SS: alternative 3′ splice site; A5′SS: alternative 5′ splice site; MXE: mutually exclusive exons; RI: retained intron; SE: skipped exon.

Inhibition of tumor growth by Ex. 6 in cell-derived xenograft (CDX) tumor models in mice.

Tumor cells were cultured using standard techniques described above and were harvested and pelleted by centrifugation at 1000 rpm for 5 minutes. The pelleted cells were washed once with serum-free medium followed by subsequent re-suspension in the serum-free medium supplemented with 50% matrigel (Corning, Inc). Five million tumor cells were implanted subcutaneously to the right flank of each mouse, followed by tumor volume and body weight measurement once or twice per week with an electronic caliper (Fowler) and a balance, respectively. When the average tumor size reached a pre-determined volume, the mice were grouped and treated with Ex. 6. One group of mice were treated with vehicle as control. During treatment, mice were monitored daily by cage side observation. The tumor volume (TV) and body weight was measured twice or three times per week during the treatment. Study ended either after 4-5 weeks of treatment or tumor volume of any mice reached 1000 or 2000 mm³. Tumor growth inhibition (TGI) was be calculated as follows:

$$100 \% * \{ 1 - [ ( T {V}_{T r e ated Last Day of Treatment} - {TV}_{Treated First Day of Treatment} ) / \\ ( {TV}_{Control on Last Day of Treatment -} {TV}_{Control on First Day of Treatment} ) ] \}$$

$$when {TV}_{Treated Last Day of Treatment} \geq {TV}_{Treated First Day of Treatment .}$$

$$In the case of {TV}_{Treated Last Day of Treatment} < {TV}_{Treated First Day of Treatment} ,$$

$$TGI was calculated as 100 \% * ( 2 - {TV}_{Treated Last Day of Treatment} / {TV}_{Treated First Day of Treatment} ) .$$

TABLE 11
Inhibition of tumor growth by Ex. 6 in CDX tumor models
with orall, once a day (QD) or other dosing schedule.

| | | | | |
|---|---|---|---|---|
| | | Treatment | | |
| Tumor | Ex. 6 Dose | Duration | | |
| Model | and Schedule | (day) | N | TGI % |

| | | | | | |
|---|---|---|---|---|---|
| MV-4-11 | 6.25 | mg/kg QD | 20 | 6 | 98.8 |
| MV-4-11 | 12.5 | mg/kg QD | 20 | 6 | 189 |
| MV-4-11 | 25 | mg/kg QD | 20 | 6 | 200 |

| | | | | |
|---|---|---|---|---|
| MV-4-11 | 25 mg/kg QD, | 20 | 6 | 200 |
| | 5dOn-2dOff | | | |

| | | | | | |
|---|---|---|---|---|---|
| MOLM-13 | 12.5 | mg/kg QD | 13 | 8 | 74 |
| MOLM-13 | 25 | mg/kg QD | 13 | 8 | 94 |
| IMR-32 | 25 | mg/kg QD | 8 | 3 | 82 |
| SW403 | 25 | mg/kg QD | 20 | 5 | 84 |
| SW480 | 25 | mg/kg QD | 28 | 4 | 93 |

What is claimed is:

1. A compound of the formula I 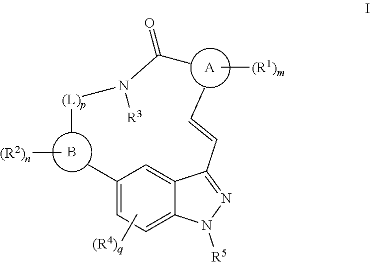
wherein
ring A is a 5- to 10-membered heteroarylene;
ring B is a 5- to 10-membered heteroarylene or C₆-C₁₀ arylene;
each L is independently —O—, —S—, —S(O)—, —S(O)₂—, —N(R⁶)C(O)—, —C(O)N(R⁶)—, —N(R⁶)—, —N(R⁶)S(O)—, —S(O)N(R⁶)—, —N(R⁶)S(O)₂—, —S(O)₂N(R⁶)—, or —C(R⁷)(R⁸)—, provided that (L)ₚ does not comprise an O—O, S—O, or N—N bond, and the point of covalent attachment of (L)ₚ to —NR³— does not form a —N—N— or a —O—N— bond;
each R¹ and R² when present, is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, is independently optionally substituted by deuterium, halogen, C₁-C₆ alkyl, C₁-C₆ haloalkyl, —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{e}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
R³ is H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR^{c}, —CN, or —NO₂;
each R⁴ is independently deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
R⁵ is H, deuterium, —C(O)R^{c}, —C(O)OR^{c}, —C(O)NR^{c}R^{d}, —P(O)₂R^{c}R^{d}, —P(O)₂NR^{c}R^{d}, —P(O)₂OR^{c}, or —S(O)₂OR′;
each R⁶, when present, is independently H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, or 5- to 10-membered heteroaryl is independently optionally substituted by —OR^{c}, —OC(O)R^{c}, —OC(O)NR^{c}R^{d}, —OC(═N)NR^{c}R^{d}, —OS(O)R^{c}, —OS(O)₂R^{c}, —OS(O)NR^{c}R^{d}, —OS(O)₂NR^{c}R^{d}, —SR^{c}, —S(O)R^{c}, —S(O)₂R^{c}, —S(O)NR^{c}R^{d}, —S(O)₂NR^{c}R^{d}, —NR^{c}R^{d}, —NR^{c}C(O)R^{d}, —N(C(O)R^{c})(C(O)R^{d}), —NR^{c}C(O)OR^{d}, —NR^{c}C(O)NR^{c}R^{d}, —NR^{c}C(═N)NR^{c}R^{d}, —NR^{c}S(O)R^{d}, —NR^{c}S(O)₂R^{d}, —NR^{c}S(O)NR^{c}R^{d}, —NR^{c}S(O)₂NR^{c}R^{d}, —C(O)R^{c}, —C(O)OR^{c}, —C(O)NR^{c}R^{d}, —C(═N)NR^{c}R^{d}, —PR^{c}R^{d}, —P(O)R^{c}R^{d}, —P(O)₂R^{c}R^{d}, —P(O)NR^{c}R^{d}, —P(O)₂NR^{c}R^{d}, —P(O)OR^{c}, —P(O)₂OR, —CN, or —NO₂;
each R⁷ and R⁸, is independently H, deuterium, halogen, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, 5- to 10-membered heteroaryl, —OR^{a}, —OC(O)R^{a}, —OC(O)NR^{a}R^{b}, —OS(O)R^{a}, —OS(O)₂R^{a}, —SR^{a}, —S(O)R^{a}, —S(O)₂R^{a}, —S(O)NR^{a}R^{b}, —S(O)₂NR^{a}R^{b}, —OS(O)NR^{a}R^{b}, —OS(O)₂NR^{a}R^{b}, —NR^{a}R^{b}, —NR^{a}C(O)R^{b}, —NR^{a}C(O)OR^{b}, —NR^{a}C(O)NR^{a}R^{b}, —NR^{a}S(O)R^{b}, —NR^{a}S(O)₂R^{b}, —NR^{a}S(O)NR^{a}R^{b}, —NR^{a}S(O)₂NR^{a}R^{b}, —C(O)R^{a}, —C(O)OR^{a}, —C(O)NR^{a}R^{b}, —PR^{a}R^{b}, —P(O)R^{a}R^{b}, —P(O)₂R^{a}R^{b}, —P(O)NR^{a}R^{b}, —P(O)₂NR^{a}R^{b}, —P(O)OR^{a}, —P(O)₂OR^{a}, —CN, or —NO₂, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, and 5- to 10-membered heteroaryl, is independently optionally substituted by deuterium, halogen, C₁-C₆ alkyl, C₁-C₆ haloalkyl, —OR^{e}, —OC(O)R^{e}, —OC(O)NR^{c}R^{f}, —OS(O)R^{c}, —OS(O)₂R^{c}, —OS(O)NR^{c}R^{f}, —OS(O)₂NR^{c}R^{f}, —SR^{c}, —S(O)R^{c}, —S(O)₂R^{c}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂; or two of R⁷ and R⁸, taken together with the carbon or carbons to which they are attached, optionally combine to form a C₃-C₆ cycloalkyl, 3- to 7-membered heterocycloalkyl, wherein each hydrogen atom in the C₃-C₆ cycloalkyl or 3- to 7-membered heterocycloalkyl formed when two of R⁷ and R⁸ are taken together is independently optionally substituted by —OR^{e}, —OC(O)R^{e}, —OC(O)NR^{e}R^{f}, —OS(O)R^{e}, —OS(O)₂R^{e}, —OS(O)NR^{e}R^{f}, —OS(O)₂NR^{e}R^{f}, —SR^{e}, —S(O)R^{e}, —S(O)₂R^{e}, —S(O)NR^{e}R^{f}, —S(O)₂NR^{e}R^{f}, —NR^{e}R^{f}, —NR^{e}C(O)R^{f}, —NR^{e}C(O)OR^{f}, —NR^{e}C(O)NR^{e}R^{f}, —NR^{e}S(O)R^{f}, —NR^{e}S(O)₂R^{f}, —NR^{e}S(O)NR^{e}R^{f}, —NR^{e}S(O)₂NR^{e}R^{f}, —C(O)R^{e}, —C(O)OR^{e}, —C(O)NR^{e}R^{f}, —PR^{e}R^{f}, —P(O)R^{e}R^{f}, —P(O)₂R^{e}R^{f}, —P(O)NR^{e}R^{f}, —P(O)₂NR^{e}R^{f}, —P(O)OR^{e}, —P(O)₂OR^{e}, —CN, or —NO₂;
each R^{a}, R^{b}, R^{c}, R^{d}, R^{e}, and R^{f} is independently selected from the group consisting of H, deuterium, C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, and C₁-C₆ alkylene-5- to 10-membered heteroaryl, or R^{a} and R^{b} or R^{e} and R^{d} or R^{e} and R^{f}, taken together with the atom to which they are attached, form a 3- to 7-membered heterocycloalkyl, wherein each hydrogen atom in C₁-C₆ alkyl, C₂-C₆ alkenyl, C₂-C₆ alkynyl, C₃-C₆ cycloalkyl, 3- to 7-membered heterocycloalkyl, C₆-C₁₀ aryl, C₁-C₆ alkylene-C₆-C₁₀ aryl, 5- to 10-membered heteroaryl, or C₁-C₆ alkylene-5- to 10-membered heteroaryl is independently optionally substituted by deuterium, halogen, C₁-C₆ alkyl, C₁-C₆ haloalkyl, —OH, —OC₁-C₆ alkyl, —OC(O)—(H or C₁-C₆ alkyl), —OC(O)N(H or C₁-C₆ alkyl)₂, —OC(O)N(C₂-C₆ alkylene), —OS(O)—(H or C₁-C₆ alkyl), —OS(O)₂—(H or C₁-C₆ alkyl), —OS(O)N(H or C₁-C₆ alkyl)₂, —OS(O)N(C₂-C₆ alkylene), —OS(O)₂N(H or C₁-C₆ alkyl)₂, —OS(O)₂N(C₂-C₆ alkylene), —S(H or C₁-C₆ alkyl), —S(O)(H or C₁-C₆ alkyl), —S(O)₂(H or C₁-C₆ alkyl), —S(O)N(H or C₁-C₆ alkyl)₂, —S(O)N(C₂-C₆ alkylene), —S(O)₂N(H or C₁-C₆ alkyl)₂, —S(O)₂N(C₂-C₆ alkylene), —N(H or C₁-C₆ alkyl)₂, —N(C₂-C₆ alkylene), —N(H or C₁-C₆ alkyl)C(O)—(H or C₁-C₆ alkyl), —N(H or C₁-C₆ alkyl)C(O)O(H or C₁-C₆ alkyl), —N(H or C₁-C₆ alkyl)C(O)N(H or C₁-C₆ alkyl)₂, —N(H or C₁-C₆ alkyl)C(O)N(C₂-C₆ alkylene), —N(H or C₁-C₆ alkyl)S(O)—(H or C₁-C₆ alkyl), —N(H or C₁-C₆ alkyl)S(O)₂(H or C₁-C₆ alkyl), —N(H or C₁-C₆ alkyl)S(O)N(H or C₁-C₆ alkyl)₂, —N(H or C₁-C₆ alkyl)S(O)N(C₂-C₆ alkylene), —N(H or C₁-C₆ alkyl)S(O)₂N(H or C₁-C₆ alkyl)₂, —N(H or C₁-C₆ alkyl)S(O)₂N(C₂-C₆ alkylene), —C(O)—(H or C₁-C₆ alkyl), —C(O)O(H or C₁-C₆ alkyl), —C(O)N(C₂-C₆ alkylene), —P(H or C₁-C₆ alkyl)₂, —P(C₂-C₆ alkylene), —P(O)(H or C₁-C₆ alkyl)₂, —P(O)(C₂-C₆ alkylene), —P(O)₂(H or C₁-C₆ alkyl)₂, —P(O)₂(C₂-C₆ alkylene), —P(O)N(H or C₁-C₆ alkyl)₂, —P(O)N(C₂-C₆ alkylene), —P(O)₂N(H or C₁-C₆ alkyl)₂, —P(O)₂N(C₂-C₆ alkylene), —P(O)O(H or C₁-C₆ alkyl), —P(O)₂O(H or C₁-C₆ alkyl), —CN, or —NO₂;
m is 0, 1, 2, or 3;
n is 0, 1, 2, 3, or 4;
p is 3, 4, 5, 6, or 7; and
q is 0, 1, or 2
or a pharmaceutically acceptable salt thereof.

2. The compound of claim 1, or a pharmaceutically acceptable salt thereof, having the formula II 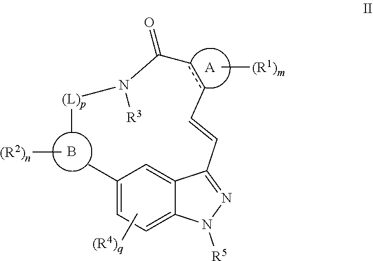
wherein “ [symbol] ” is optionally a carbon-carbon single bond or a carbon-carbon double bond, and ring A is a 5-membered heteroarylene.

3. The compound of claim 1 or 2, or a pharmaceutically acceptable salt thereof, having the formula III 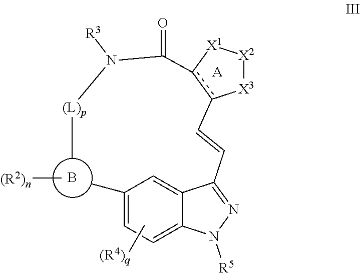
wherein
X¹, X², and X³ are each independently —O—, —S—, ═C(H)—, ═C(R¹)—, —N(H)—, —N(R¹)— or ═N— and ring A is a 5-membered heteroarylene, provided that at least one of X¹, X², and X³ is not ═C(H)—, or ═C(R¹)—; and
“ [symbol] ” is optionally a carbon-carbon single bond or a carbon-carbon double bond.

4. The compound of any one of the preceding claims, or a pharmaceutically acceptable salt thereof, wherein ring A is a 5-membered heteroarylene selected from the group consisting of 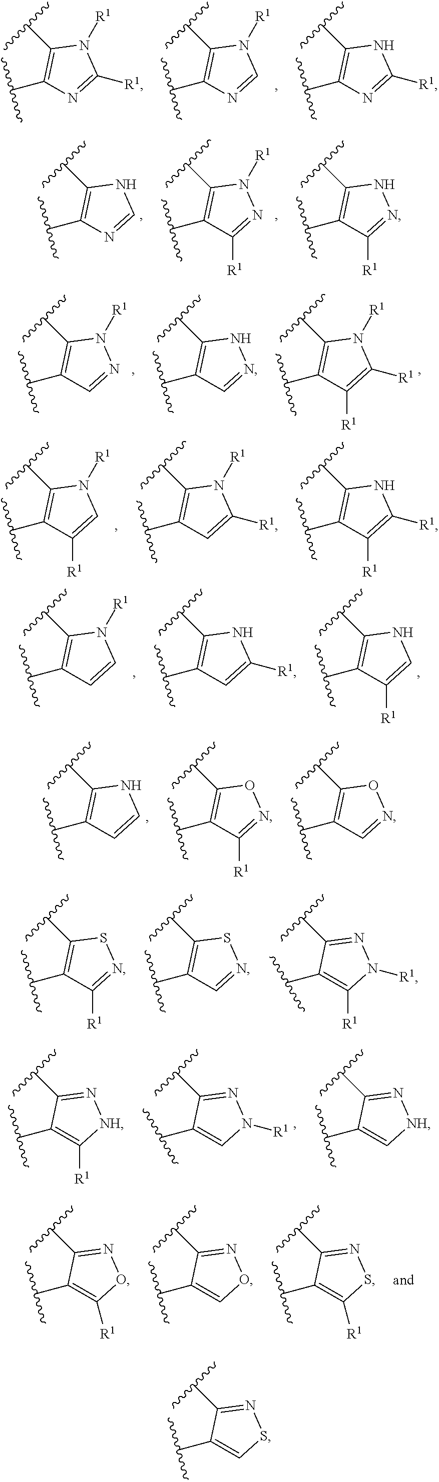
wherein each “ [symbol] ” represents a point of covalent attachment.

5. The compound of any one of the preceding claims, or a pharmaceutically acceptable salt thereof, wherein ring A is a 5-membered heteroarylene selected from the group consisting of 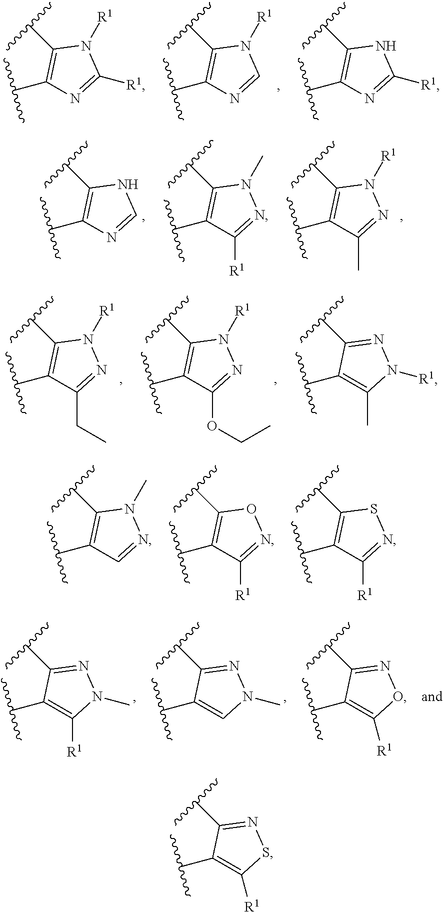
wherein each “ [symbol] ” represents a point of covalent attachment.

6. The compound of any one of the preceding claims, or a pharmaceutically acceptable salt thereof, wherein ring A is a 5-membered heteroarylene selected from the group consisting of 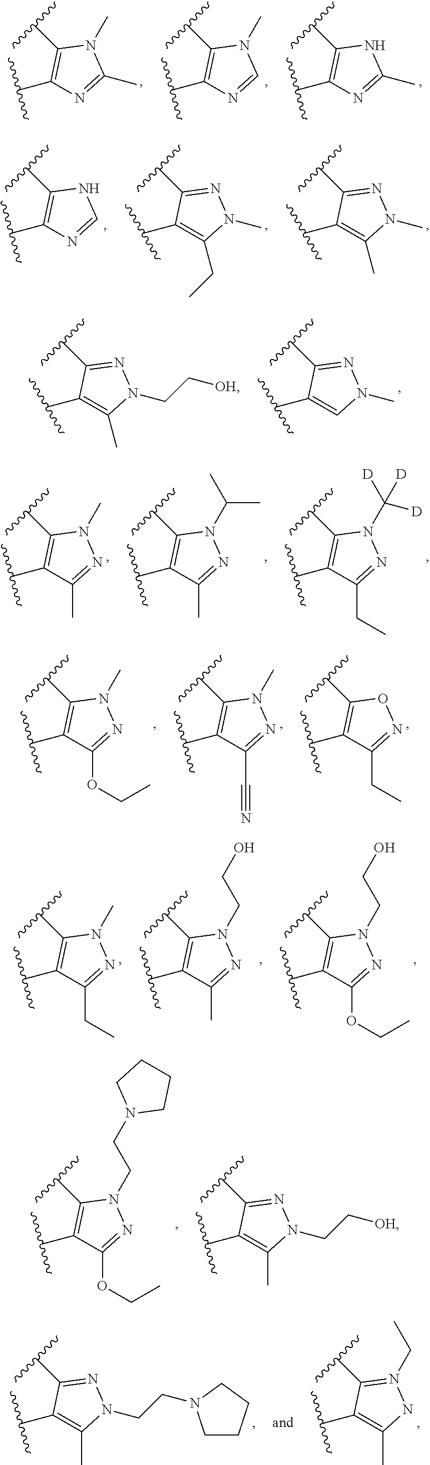
wherein each “ [symbol] ” represents a point of covalent attachment.

7. The compound of any one of the preceding claims, or a pharmaceutically acceptable salt thereof, wherein ring B is a C₆-C₁₀ arylene, and n is 0, 1, or 2.

8. The compound of any one of the preceding claims, or a pharmaceutically acceptable salt thereof, wherein ring B is a phenylene, and n is 0, 1, or 2.

9. The compound of any one of the preceding claims, or a pharmaceutically acceptable salt thereof, wherein ring B is a phenylene, and n is 0 or 1.

10. The compound of any one of the preceding claims, or a pharmaceutically acceptable salt thereof, wherein ring B is a phenylene, n is 1, and R² is methyl, ethyl, F, Cl, or Br.

11. The compound of any one of the preceding claims, or a pharmaceutically acceptable salt thereof, wherein ring B is 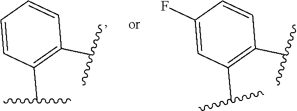 [SMILES: *c1ccccc1* ; *c1ccc(F)cc1*]
wherein each “ [symbol] ” represents a point of covalent attachment.

12. The compound of any one of claims 1 to 6, or a pharmaceutically acceptable salt thereof, wherein ring B is a 5- to 10-membered heteroarylene.

13. The compound of any one of claims 1 to 6 or 12, or a pharmaceutically acceptable salt thereof, wherein ring B is a 5-membered heteroarylene selected from the group consisting of 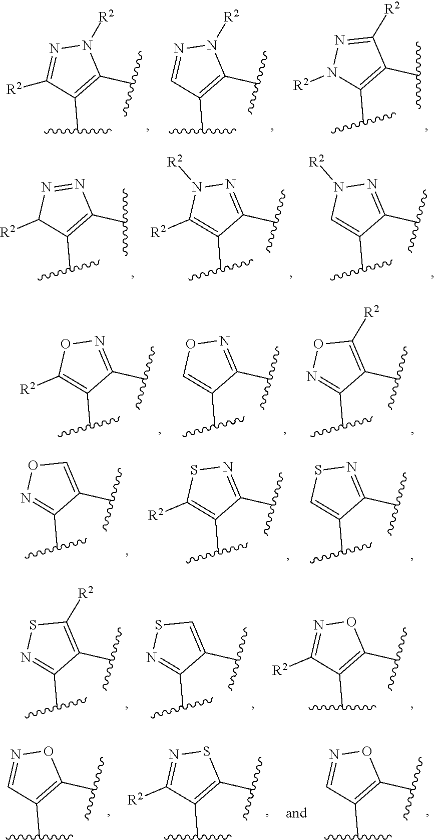
wherein each “ [symbol] ” represents a point of covalent attachment.

14. The compound of any one of claims 1 to 6, 12, or 13, or a pharmaceutically acceptable salt thereof, wherein ring B is a 5-membered heteroarylene selected from the group consisting of 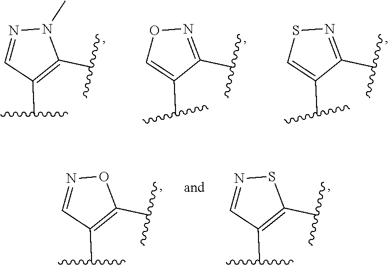
wherein each “ [symbol] ” represents a point of covalent attachment.

15. The compound of any one of the preceding claims, or a pharmaceutically acceptable salt thereof, wherein R³ is H or methyl.

16. The compound of any one of the preceding claims, or a pharmaceutically acceptable salt thereof, wherein R⁴ is H or methyl.

17. The compound of any one of the preceding claims, or a pharmaceutically acceptable salt thereof, wherein R⁵ is H.

18. The compound of any one of the preceding claims, or a pharmaceutically acceptable salt thereof, wherein each L is independently each L is independently —C(R⁷)(R⁸)—, —C(O)—, —O—, or —N(R⁶)—, provided that (L)ₚ does not comprise a —O—O— or a —O—N(R⁶)— bond, and the point of covalent attachment of (L)ₚ to —NR³— does not form a —N—N— or a —O—N— bond.

19. The compound of any one of the preceding claims, or a pharmaceutically acceptable salt thereof, wherein -(L)ₚ- is —(CR⁷R⁸)C(O)N(R⁶)—(CR⁷R⁸)₂—, —(CR⁷R⁸)N(R⁶)C(O)—(CR⁷R⁸)₂—, —N(R⁶)—C(O)(CR⁷R⁸)₂O(CR⁷R⁸)₂—, —CR⁷R⁸O(CR⁷R⁸)₂O—(CR⁷R⁸)₂, —O(CR⁷R⁸)₂O(CR⁷R⁸)₂—, —CR⁷R⁸O—CR⁷R⁸—C(O)N(R⁶)—(CR⁷R⁸)₂—, —(CR⁷R⁸)₃O(CR⁷R⁸)₂—, —(CR⁷R⁸)₂O(CR⁷R⁸)₃—, —CR⁷R⁸—N(R⁶)—(CR⁷R⁸)₂—, —CR⁷R⁸—N(R⁶)—(CR⁷R⁸)₃—, —O(CR⁷R⁸)₂O(CR⁷R⁸)₃—, —(CR⁷R⁸)₂—N(R⁶)—(CR⁷R⁸)₃—, —(CR⁷R⁸)₂—N(R⁶)—(CR⁷R⁸)₂—, —O—(CR⁷R⁸)₂—, —O—(CR⁷R⁸)₃—, or —O—(CR⁷R⁸)₄—.

20. The compound of any one of the preceding claims, or a pharmaceutically acceptable salt thereof, wherein R⁶ is H or methyl.

21. The compound of any one of the preceding claims, or a pharmaceutically acceptable salt thereof, wherein each R⁷ and R⁸ is H.

22. The compound of any one of the preceding claims, or a pharmaceutically acceptable salt thereof, wherein -(L)ₚ- is —CH₂N(H)—(CH₂)₂—, —CH₂N(CH₃)—(CH₂)₂—, —O(CH₂)₂—, —OCH(CH₃)CH₂—, —O(CH₂)₃—, —O(CH₂)₄—, and —O(CH₂)₂O(CH₂)₂—.

23. A compound selected from the group consisting of 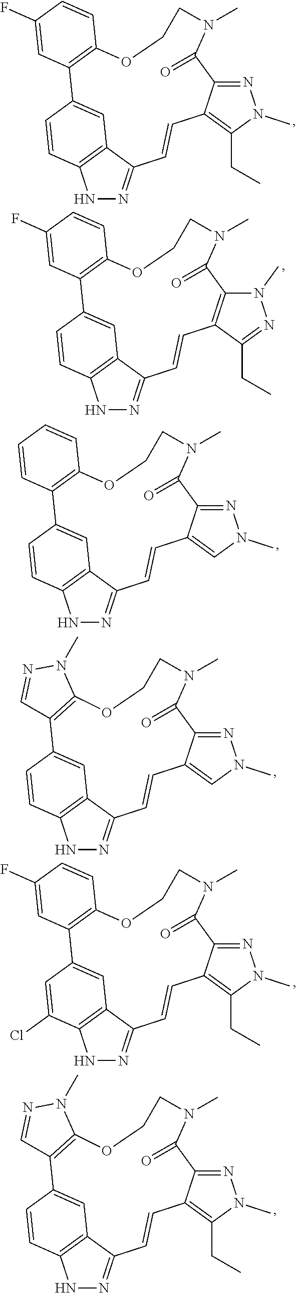 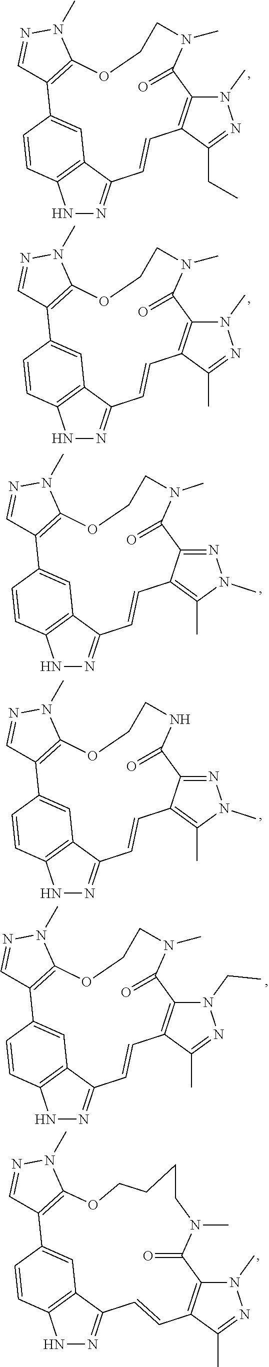 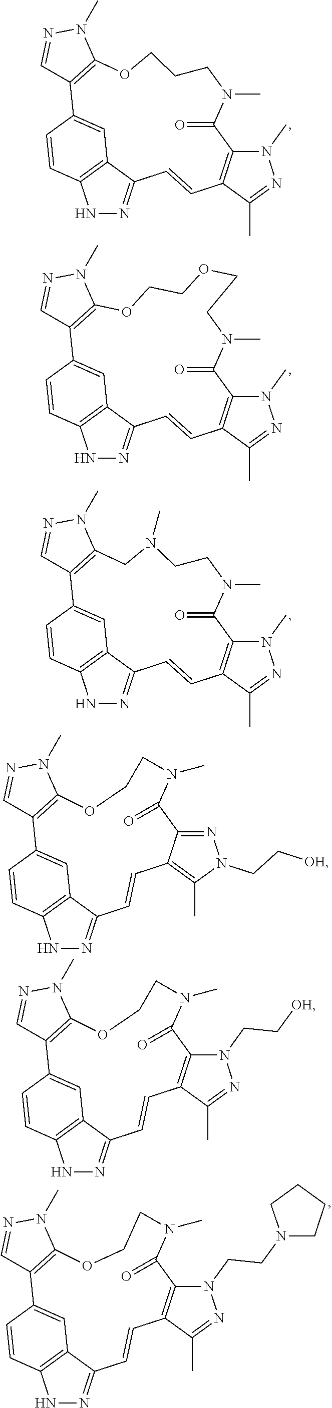 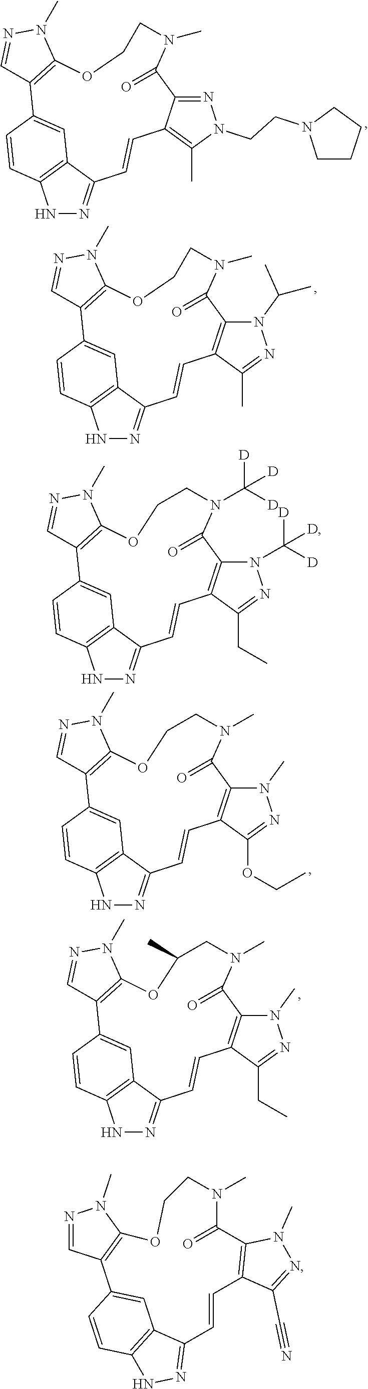 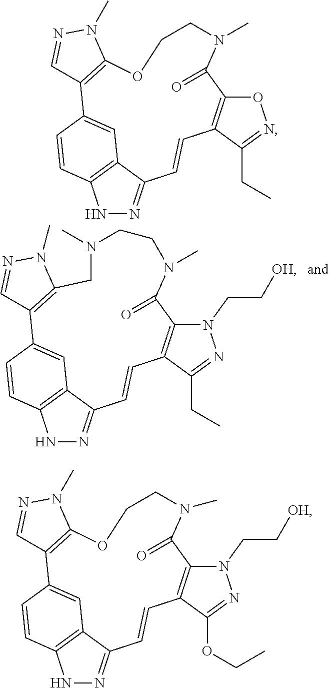
or a pharmaceutically acceptable salt thereof.

24. A pharmaceutical composition comprising a compound of any one of the preceding claims, and optionally one or more excipients.

25. A method of treating disease in a subject comprising, administering a therapeutically effective amount of a compound of any one of claims 1 to 23, or a pharmaceutical composition of claim 24.

26. A compound according to any one of claims 1 to 23, for use in a method of treating disease in a subject.

27. Use of a compound according to any one of claims 1 to 23 in the manufacture of a medicament for the treatment of disease in a subject.
